SEQUENCE LISTING (60 sequences)

SEQ ID NO: 1 - protein, 15 aa, synthetic construct
GGGGSGGGGSGGGGS

SEQ ID NO: 2 - protein, 18 aa, synthetic construct
GGSSRSSSSGGGGSGGGG

SEQ ID NO: 3 - protein, 21 aa, synthetic construct
SPNSASHSGSAPQTSSAPGSQ

SEQ ID NO: 4 - protein, 19 aa, synthetic construct
EPKSSDKTHTSPPSPAPEL

SEQ ID NO: 5 - protein, 13 aa, synthetic construct
GGGGSHHHHHHHH

SEQ ID NO: 6 - protein, 529 aa, synthetic construct
QIQLVQSGPELKKPGETVKISCKASGYTFKNYGMNWVKQAPGKGLKWMGWINTYTGQPIYANDFKGRFAFSLETSASTAYLQINNLKNEDTATYFCARDWGPYWGQGTLVIVSASPNSASHSGSAPQTSSAPGSQNIMMTQSPSSLAVSAGEKVTVNCKSSQSVLYSSNQMNYLAWYQQKPGQSPKLLIYWASTRESGVPDRFTGSGSGTDFTLTISSVQTEDLAVYYCLQYLSSWTFGGGTKLEIKGGSSRSSSSGGGGSGGGGEVQLLESGGGLVQPGGSLRLSCAASGFTFSSFPMAWVRQAPGKGLEWVSTISTSGGRTYYRDSVKGRFTISRDNSKNTLYLQMNSLRAEDTAVYYCAKFRQYSGGFDYWGQGTLVTVSSSPNSASHSGSAPQTSSAPGSQDIQLTQPNSVSTSLGSTVKLSCTLSSGNIENNYVHWYQLYEGRSPTTMIYDDDKRPDGVPDRFSGSIDRSSNSAFLTIHNVAIEDEAIYFCHSYVSSFNVFGGGTKLTVLRGGGGSHHHHHHHH

SEQ ID NO: 7 - protein, 383 aa, synthetic construct
AGQPLQERAQAWGERLRARMEEMGSRTRDRLDEVKEQVAEVRAKLEEQAQQIRLQAEAAQARLKSRFEPLAEDMQRQWAGQVEKVQAAEGTSAAPVPSDNHGGSSRSSSSGGGGSGGGGEVQLLESGGGLVQPGGSLRLSCAASGFTFSSFPMAWVRQAPGKGLEWVSTISTSGGRTYYRDSVKGRFTISRDNSKNTLYLQMNSLRAEDTAVYYCAKFRQYSGGFDYWGQGTLVTVSSSPNSASHSGSAPQTSSAPGSQDIQLTQPNSVSTSLGSTVKLSCTLSSGNIENNYVHWYQLYEGRSPTTMIYDDDKRPDGVPDRFSGSIDRSSNSAFLTIHNVAIEDEAIYFCHSYVSSFNVFGGGTKLTVLRGGGGSHHHHHHHH

SEQ ID NO: 8 - protein, 648 aa, synthetic construct
QIQLVQSGPELKKPGETVKISCKASGYTFKNYGMNWVKQAPGKGLKWMGWINTYTGQPIYANDFKGRFAFSLETSASTAYLQINNLKNEDTATYFCARDWGPYWGQGTLVIVSASPNSASHSGSAPQTSSAPGSQNIMMTQSPSSLAVSAGEKVTVNCKSSQSVLYSSNQMNYLAWYQQKPGQSPKLLIYWASTRESGVPDRFTGSGSGTDFTLTISSVQTEDLAVYYCLQYLSSWTFGGGTKLEIKGGSSRSSSSGGGGSGGGGAGQPLQERAQAWGERLRARMEEMGSRTRDRLDEVKEQVAEVRAKLEEQAQQIRLQAEAAQARLKSRFEPLAEDMQRQWAGQVEKVQAAEGTSAAPVPSDNHGGSSRSSSSGGGGSGGGGEVQLLESGGGLVQPGGSLRLSCAASGFTFSSFPMAWVRQAPGKGLEWVSTISTSGGRTYYRDSVKGRFTISRDNSKNTLYLQMNSLRAEDTAVYYCAKFRQYSGGFDYWGQGTLVTVSSSPNSASHSGSAPQTSSAPGSQDIQLTQPNSVSTSLGSTVKLSCTLSSGNIENNYVHWYQLYEGRSPTTMIYDDDKRPDGVPDRFSGSIDRSSNSAFLTIHNVAIEDEAIYFCHSYVSSFNVFGGGTKLTVLRGGGGSHHHHHHHH

SEQ ID NO: 9 - protein, 581 aa, synthetic construct
KVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASALRALRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNHGGSSRSSSSGGGGSGGGGEVQLLESGGGLVQPGGSLRLSCAASGFTFSSFPMAWVRQAPGKGLEWVSTISTSGGRTYYRDSVKGRFTISRDNSKNTLYLQMNSLRAEDTAVYYCAKFRQYSGGFDYWGQGTLVTVSSSPNSASHSGSAPQTSSAPGSQDIQLTQPNSVSTSLGSTVKLSCTLSSGNIENNYVHWYQLYEGRSPTTMIYDDDKRPDGVPDRFSGSIDRSSNSAFLTIHNVAIEDEAIYFCHSYVSSFNVFGGGTKLTVLRGGGGSHHHHHHHH

SEQ ID NO: 10 - protein, 846 aa, synthetic construct
QIQLVQSGPELKKPGETVKISCKASGYTFKNYGMNWVKQAPGKGLKWMGWINTYTGQPIYANDFKGRFAFSLETSASTAYLQINNLKNEDTATYFCARDWGPYWGQGTLVIVSASPNSASHSGSAPQTSSAPGSQNIMMTQSPSSLAVSAGEKVTVNCKSSQSVLYSSNQMNYLAWYQQKPGQSPKLLIYWASTRESGVPDRFTGSGSGTDFTLTISSVQTEDLAVYYCLQYLSSWTFGGGTKLEIKGGSSRSSSSGGGGSGGGGKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASALRALRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNHGGSSRSSSSGGGGSGGGGEVQLLESGGGLVQPGGSLRLSCAASGFTFSSFPMAWVRQAPGKGLEWVSTISTSGGRTYYRDSVKGRFTISRDNSKNTLYLQMNSLRAEDTAVYYCAKFRQYSGGFDYWGQGTLVTVSSSPNSASHSGSAPQTSSAPGSQDIQLTQPNSVSTSLGSTVKLSCTLSSGNIENNYVHWYQLYEGRSPTTMIYDDDKRPDGVPDRFSGSIDRSSNSAFLTIHNVAIEDEAIYFCHSYVSSFNVFGGGTKLTVLRGGGGSHHHHHHHH

SEQ ID NO: 11 - protein, 535 aa, synthetic construct
QIQLVQSGPELKKPGETVKISCKASGYTFKNYGMNWVKQAPGKGLKWMGWINTYTGQPIYANDFKGRFAFSLETSASTAYLQINNLKNEDTATYFCARDWGPYWGQGTLVIVSASPNSASHSGSAPQTSSAPGSQNIMMTQSPSSLAVSAGEKVTVNCKSSQSVLYSSNQMNYLAWYQQKPGQSPKLLIYWASTRESGVPDRFTGSGSGTDFTLTISSVQTEDLAVYYCLQYLSSWTFGGGTKLEIKGGSSRSSSSGGGGSGGGGEVRLQQSGPDLIKPGASVKMSCKASGYTFTGYVMHWVKQRPGQGLEWIGFINPYNDDIQSNERFRGKATLTSDKSSTTAYMELSSLTSEDSAVYYCARGAGYNFDGAYRFFDFWGQGTTLTVSSSPNSASHSGSAPQTSSAPGSQDVVMTQSPLSLPVSLGDQASISCRSSQRLVHSNGNTYLHWYLQKPGQSPKLLIYRVSNRFPGVPDRFSGSGSGTDFTLKISRVEAEDLGIYFCSQSTHVPYTFGGGTKLEIKGGGGSHHHHHHHH

SEQ ID NO: 12 - protein, 389 aa, synthetic construct
AGQPLQERAQAWGERLRARMEEMGSRTRDRLDEVKEQVAEVRAKLEEQAQQIRLQAEAAQARLKSRFEPLAEDMQRQWAGQVEKVQAAEGTSAAPVPSDNHGGSSRSSSSGGGGSGGGGEVRLQQSGPDLIKPGASVKMSCKASGYTFTGYVMHWVKQRPGQGLEWIGFINPYNDDIQSNERFRGKATLTSDKSSTTAYMELSSLTSEDSAVYYCARGAGYNFDGAYRFFDFWGQGTTLTVSSSPNSASHSGSAPQTSSAPGSQDVVMTQSPLSLPVSLGDQASISCRSSQRLVHSNGNTYLHWYLQKPGQSPKLLIYRVSNRFPGVPDRFSGSGSGTDFTLKISRVEAEDLGIYFCSQSTHVPYTFGGGTKLEIKGGGGSHHHHHHHH

SEQ ID NO: 13 - protein, 654 aa, synthetic construct
QIQLVQSGPELKKPGETVKISCKASGYTFKNYGMNWVKQAPGKGLKWMGWINTYTGQPIYANDFKGRFAFSLETSASTAYLQINNLKNEDTATYFCARDWGPYWGQGTLVIVSASPNSASHSGSAPQTSSAPGSQNIMMTQSPSSLAVSAGEKVTVNCKSSQSVLYSSNQMNYLAWYQQKPGQSPKLLIYWASTRESGVPDRFTGSGSGTDFTLTISSVQTEDLAVYYCLQYLSSWTFGGGTKLEIKGGSSRSSSSGGGGSGGGGAGQPLQERAQAWGERLRARMEEMGSRTRDRLDEVKEQVAEVRAKLEEQAQQIRLQAEAAQARLKSRFEPLAEDMQRQWAGQVEKVQAAEGTSAAPVPSDNHGGSSRSSSSGGGGSGGGGEVRLQQSGPDLIKPGASVKMSCKASGYTFTGYVMHWVKQRPGQGLEWIGFINPYNDDIQSNERFRGKATLTSDKSSTTAYMELSSLTSEDSAVYYCARGAGYNFDGAYRFFDFWGQGTTLTVSSSPNSASHSGSAPQTSSAPGSQDVVMTQSPLSLPVSLGDQASISCRSSQRLVHSNGNTYLHWYLQKPGQSPKLLIYRVSNRFPGVPDRFSGSGSGTDFTLKISRVEAEDLGIYFCSQSTHVPYTFGGGTKLEIKGGGGSHHHHHHHH

SEQ ID NO: 14 - protein, 587 aa, synthetic construct
KVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASALRALRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNHGGSSRSSSSGGGGSGGGGEVRLQQSGPDLIKPGASVKMSCKASGYTFTGYVMHWVKQRPGQGLEWIGFINPYNDDIQSNERFRGKATLTSDKSSTTAYMELSSLTSEDSAVYYCARGAGYNFDGAYRFFDFWGQGTTLTVSSSPNSASHSGSAPQTSSAPGSQDVVMTQSPLSLPVSLGDQASISCRSSQRLVHSNGNTYLHWYLQKPGQSPKLLIYRVSNRFPGVPDRFSGSGSGTDFTLKISRVEAEDLGIYFCSQSTHVPYTFGGGTKLEIKGGGGSHHHHHHHH

SEQ ID NO: 15 - protein, 852 aa, synthetic construct
QIQLVQSGPELKKPGETVKISCKASGYTFKNYGMNWVKQAPGKGLKWMGWINTYTGQPIYANDFKGRFAFSLETSASTAYLQINNLKNEDTATYFCARDWGPYWGQGTLVIVSASPNSASHSGSAPQTSSAPGSQNIMMTQSPSSLAVSAGEKVTVNCKSSQSVLYSSNQMNYLAWYQQKPGQSPKLLIYWASTRESGVPDRFTGSGSGTDFTLTISSVQTEDLAVYYCLQYLSSWTFGGGTKLEIKGGSSRSSSSGGGGSGGGGKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASALRALRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNHGGSSRSSSSGGGGSGGGGEVRLQQSGPDLIKPGASVKMSCKASGYTFTGYVMHWVKQRPGQGLEWIGFINPYNDDIQSNERFRGKATLTSDKSSTTAYMELSSLTSEDSAVYYCARGAGYNFDGAYRFFDFWGQGTTLTVSSSPNSASHSGSAPQTSSAPGSQDVVMTQSPLSLPVSLGDQASISCRSSQRLVHSNGNTYLHWYLQKPGQSPKLLIYRVSNRFPGVPDRFSGSGSGTDFTLKISRVEAEDLGIYFCSQSTHVPYTFGGGTKLEIKGGGGSHHHHHHHH

SEQ ID NO: 16 - protein, 524 aa, synthetic construct
QIQLVQSGPELKKPGETVKISCKASGYTFKNYGMNWVKQAPGKGLKWMGWINTYTGQPIYANDFKGRFAFSLETSASTAYLQINNLKNEDTATYFCARDWGPYWGQGTLVIVSASPNSASHSGSAPQTSSAPGSQNIMMTQSPSSLAVSAGEKVTVNCKSSQSVLYSSNQMNYLAWYQQKPGQSPKLLIYWASTRESGVPDRFTGSGSGTDFTLTISSVQTEDLAVYYCLQYLSSWTFGGGTKLEIKGGSSRSSSSGGGGSGGGGQIQLVQSGPGLKKPGGSVRISCAASGYTFTNYGMNWVKQAPGKGLRWMGWINTHTGEPTYADDFKGRFTFSLDTSKSTAYLQINSLRAEDTATYFCTRRGYDWYFDVWGQGTTVTVSSSPNSASHSGSAPQTSSAPGSQDIQMTQSPSSMSASLGDRVTITCRASQDINSYLSWFQQKPGKSPKTLIYRANRLVDGVPSRFSGSGSGTDYTLTISSLQYEDFGIYYCQQYDESPWTFGGGTKLEIKGGGGSHHHHHHHH

SEQ ID NO: 17 - protein, 378 aa, synthetic construct
AGQPLQERAQAWGERLRARMEEMGSRTRDRLDEVKEQVAEVRAKLEEQAQQIRLQAEAAQARLKSRFEPLAEDMQRQWAGQVEKVQAAEGTSAAPVPSDNHGGSSRSSSSGGGGSGGGGQIQLVQSGPGLKKPGGSVRISCAASGYTFTNYGMNWVKQAPGKGLRWMGWINTHTGEPTYADDFKGRFTFSLDTSKSTAYLQINSLRAEDTATYFCTRRGYDWYFDVWGQGTTVTVSSSPNSASHSGSAPQTSSAPGSQDIQMTQSPSSMSASLGDRVTITCRASQDINSYLSWFQQKPGKSPKTLIYRANRLVDGVPSRFSGSGSGTDYTLTISSLQYEDFGIYYCQQYDESPWTFGGGTKLEIKGGGGSHHHHHHHH

SEQ ID NO: 18 - protein, 643 aa, synthetic construct
QIQLVQSGPELKKPGETVKISCKASGYTFKNYGMNWVKQAPGKGLKWMGWINTYTGQPIYANDFKGRFAFSLETSASTAYLQINNLKNEDTATYFCARDWGPYWGQGTLVIVSASPNSASHSGSAPQTSSAPGSQNIMMTQSPSSLAVSAGEKVTVNCKSSQSVLYSSNQMNYLAWYQQKPGQSPKLLIYWASTRESGVPDRFTGSGSGTDFTLTISSVQTEDLAVYYCLQYLSSWTFGGGTKLEIKGGSSRSSSSGGGGSGGGGAGQPLQERAQAWGERLRARMEEMGSRTRDRLDEVKEQVAEVRAKLEEQAQQIRLQAEAAQARLKSRFEPLAEDMQRQWAGQVEKVQAAEGTSAAPVPSDNHGGSSRSSSSGGGGSGGGGQIQLVQSGPGLKKPGGSVRISCAASGYTFTNYGMNWVKQAPGKGLRWMGWINTHTGEPTYADDFKGRFTFSLDTSKSTAYLQINSLRAEDTATYFCTRRGYDWYFDVWGQGTTVTVSSSPNSASHSGSAPQTSSAPGSQDIQMTQSPSSMSASLGDRVTITCRASQDINSYLSWFQQKPGKSPKTLIYRANRLVDGVPSRFSGSGSGTDYTLTISSLQYEDFGIYYCQQYDESPWTFGGGTKLEIKGGGGSHHHHHHHH

SEQ ID NO: 19 - protein, 576 aa, synthetic construct
KVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASALRALRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNHGGSSRSSSSGGGGSGGGGQIQLVQSGPGLKKPGGSVRISCAASGYTFTNYGMNWVKQAPGKGLRWMGWINTHTGEPTYADDFKGRFTFSLDTSKSTAYLQINSLRAEDTATYFCTRRGYDWYFDVWGQGTTVTVSSSPNSASHSGSAPQTSSAPGSQDIQMTQSPSSMSASLGDRVTITCRASQDINSYLSWFQQKPGKSPKTLIYRANRLVDGVPSRFSGSGSGTDYTLTISSLQYEDFGIYYCQQYDESPWTFGGGTKLEIKGGGGSHHHHHHHH

SEQ ID NO: 20 - protein, 841 aa, synthetic construct
QIQLVQSGPELKKPGETVKISCKASGYTFKNYGMNWVKQAPGKGLKWMGWINTYTGQPIYANDFKGRFAFSLETSASTAYLQINNLKNEDTATYFCARDWGPYWGQGTLVIVSASPNSASHSGSAPQTSSAPGSQNIMMTQSPSSLAVSAGEKVTVNCKSSQSVLYSSNQMNYLAWYQQKPGQSPKLLIYWASTRESGVPDRFTGSGSGTDFTLTISSVQTEDLAVYYCLQYLSSWTFGGGTKLEIKGGSSRSSSSGGGGSGGGGKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASALRALRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNHGGSSRSSSSGGGGSGGGGQIQLVQSGPGLKKPGGSVRISCAASGYTFTNYGMNWVKQAPGKGLRWMGWINTHTGEPTYADDFKGRFTFSLDTSKSTAYLQINSLRAEDTATYFCTRRGYDWYFDVWGQGTTVTVSSSPNSASHSGSAPQTSSAPGSQDIQMTQSPSSMSASLGDRVTITCRASQDINSYLSWFQQKPGKSPKTLIYRANRLVDGVPSRFSGSGSGTDYTLTISSLQYEDFGIYYCQQYDESPWTFGGGTKLEIKGGGGSHHHHHHHH

SEQ ID NO: 21 - protein, 527 aa, synthetic construct
QIQLVQSGPELKKPGETVKISCKASGYTFKNYGMNWVKQAPGKGLKWMGWINTYTGQPIYANDFKGRFAFSLETSASTAYLQINNLKNEDTATYFCARDWGPYWGQGTLVIVSASPNSASHSGSAPQTSSAPGSQNIMMTQSPSSLAVSAGEKVTVNCKSSQSVLYSSNQMNYLAWYQQKPGQSPKLLIYWASTRESGVPDRFTGSGSGTDFTLTISSVQTEDLAVYYCLQYLSSWTFGGGTKLEIKGGSSRSSSSGGGGSGGGGQVKLQQSGPELKKPGETVKISCKASGYTFTNYGMNWVKQAPGKGLKWMGWINTYTGESTYADDFKGRFAFSLETSASAAYLQINNLKNEDTATYFCARFAIKGDYWGQGTTVTVSSSPNSASHSGSAPQTSSAPGSQDIVLTQSPFSNPVTLGTSASISCRSTKSLLHSNGITYLYWYLQKPGQSPQLLIYQMSNLASGVPDRFSSSGSGTDFTLRISRVEAEDVGVYYCAQNLEIPRTFGGGTKLEIKGGGGSHHHHHHHH

SEQ ID NO: 22 - DNA, 1587 bp, synthetic construct
  source 1..1587: mol_type = other DNA
cagatccagctggtgcagtccggcccagagctgaaaaagccaggcgagaccgtgaagatctcctgcaaggccagcggctacactttcaagaattatggcatgaactgggtgaagcaggcccccggcaaggggctgaagtggatggggtggattaacacctataccggccagcccatttacgccaacgatttcaaggggagattcgcctttagcctggaaaccagcgcgagcaccgcctatctgcagatcaacaatcttaagaatgaggatacagccacctacttttgcgcccgggattggggcccatactggggccagggcaccctggtgatcgtgtccgcttcccccaattctgcctcccacagcggcagcgccccacagactagctccgcccccggttcccagaatatcatgatgacacagagcccttccagcctggccgtgtctgccggcgaaaaggtgaccgtgaactgcaagtctagccagtctgtgctgtacagctccaatcagatgaactatctggcctggtaccagcagaagcctggccagagccctaagctgctgatttactgggccagcacacgcgagtctggggtccctgacagatttaccggtagcggaagcggcacagatttcacactgacaatctcttctgtgcagaccgaagacctcgcagtgtactactgtctgcagtatctgagctcttggactttcggcggggggaccaagctggaaattaagggtggaagcagtagatcatccagttctggtgggggaggatccgggggcgggggtgaggttcagctccttgaatcaggcggagggctcgtccaaccaggcggatctttgcggctgtcatgtgccgcatccggattcacgttctcctcctttccgatggcatgggtgaggcaagctcctggaaaaggtctcgagtgggtatccaccataagtacttctggcgggcgcacttactacagggatagtgtgaagggaaggtttaccatttccagagataactcaaagaataccctgtacctgcaaatgaactccctcagggctgaagatacggctgtgtactactgtgctaaatttcgacaatactcaggtgggtttgactattggggccaggggacactggtgactgtgtcatcaagtcccaattccgcctcacatagcggaagtgccccacaaacttcctccgcgccaggttctcaagacattcaattgacgcagccgaatagcgtctctacgagcttggggagtaccgttaagctttcatgtactctttcctccggtaacatagagaacaactacgtgcattggtaccaactctacgagggcaggtctccaacgactatgatttacgatgacgataaacgacctgatggggtccctgaccgcttttcaggaagcattgacagaagctctaattccgcgtttttgactatccacaacgtagcgattgaagatgaagcaatttacttctgtcattcctatgtatccagctttaacgtcttcggaggtggaactaagttgacggtactccgagggggaggtgggtctcaccaccatcaccaccaccatcat

SEQ ID NO: 23 - DNA, 1149 bp, synthetic construct
  source 1..1149: mol_type = other DNA
gctggacagccgctgcaagagcgggcacaagcctggggggaaagactcagggccagaatggaagaaatgggatcaagaacaagggacaggctcgatgaggtgaaggagcaagtagcagaggttcgagcaaagctggaagaacaagctcagcagattagattgcaagcagaggcggctcaagcacgactcaagagcaggttcgagccattggctgaagatatgcagcgacaatgggctggtcaggttgagaaagtccaagcggcggaagggaccagtgcagcaccggtaccatccgacaatcatggtggaagcagtagatcatccagttctggtgggggaggatccgggggcgggggtgaggttcagctccttgaatcaggcggagggctcgtccaaccaggcggatctttgcggctgtcatgtgccgcatccggattcacgttctcctcctttccgatggcatgggtgaggcaagctcctggaaaaggtctcgagtgggtatccaccataagtacttctggcgggcgcacttactacagggatagtgtgaagggaaggtttaccatttccagagataactcaaagaataccctgtacctgcaaatgaactccctcagggctgaagatacggctgtgtactactgtgctaaatttcgacaatactcaggtgggtttgactattggggccaggggacactggtgactgtgtcatcaagtcccaattccgcctcacatagcggaagtgccccacaaacttcctccgcgccaggttctcaagacattcaattgacgcagccgaatagcgtctctacgagcttggggagtaccgttaagctttcatgtactctttcctccggtaacatagagaacaactacgtgcattggtaccaactctacgagggcaggtctccaacgactatgatttacgatgacgataaacgacctgatggggtccctgaccgcttttcaggaagcattgacagaagctctaattccgcgtttttgactatccacaacgtagcgattgaagatgaagcaatttacttctgtcattcctatgtatccagctttaacgtcttcggaggtggaactaagttgacggtactccgagggggaggtgggtctcaccaccatcaccaccaccatcat

SEQ ID NO: 24 - DNA, 1944 bp, synthetic construct
  source 1..1944: mol_type = other DNA
cagatccagctggtgcagtccggcccagagctgaaaaagccaggcgagaccgtgaagatctcctgcaaggccagcggctacactttcaagaattatggcatgaactgggtgaagcaggcccccggcaaggggctgaagtggatggggtggattaacacctataccggccagcccatttacgccaacgatttcaaggggagattcgcctttagcctggaaaccagcgcgagcaccgcctatctgcagatcaacaatcttaagaatgaggatacagccacctacttttgcgcccgggattggggcccatactggggccagggcaccctggtgatcgtgtccgcttcccccaattctgcctcccacagcggcagcgccccacagactagctccgcccccggttcccagaatatcatgatgacacagagcccttccagcctggccgtgtctgccggcgaaaaggtgaccgtgaactgcaagtctagccagtctgtgctgtacagctccaatcagatgaactatctggcctggtaccagcagaagcctggccagagccctaagctgctgatttactgggccagcacacgcgagtctggggtccctgacagatttaccggtagcggaagcggcacagatttcacactgacaatctcttctgtgcagaccgaagacctcgcagtgtactactgtctgcagtatctgagctcttggactttcggcggggggaccaagctggaaattaagggcggatctagtcggtcatcctccagtggtggtgggggtagcgggggaggcggtgctggacagccgctgcaagagcgggcacaagcctggggggaaagactcagggccagaatggaagaaatgggatcaagaacaagggacaggctcgatgaggtgaaggagcaagtagcagaggttcgagcaaagctggaagaacaagctcagcagattagattgcaagcagaggcggctcaagcacgactcaagagcaggttcgagccattggctgaagatatgcagcgacaatgggctggtcaggttgagaaagtccaagcggcggaagggaccagtgcagcaccggtaccatccgacaatcatggtggaagcagtagatcatccagttctggtgggggaggatccgggggcgggggtgaggttcagctccttgaatcaggcggagggctcgtccaaccaggcggatctttgcggctgtcatgtgccgcatccggattcacgttctcctcctttccgatggcatgggtgaggcaagctcctggaaaaggtctcgagtgggtatccaccataagtacttctggcgggcgcacttactacagggatagtgtgaagggaaggtttaccatttccagagataactcaaagaataccctgtacctgcaaatgaactccctcagggctgaagatacggctgtgtactactgtgctaaatttcgacaatactcaggtgggtttgactattggggccaggggacactggtgactgtgtcatcaagtcccaattccgcctcacatagcggaagtgccccacaaacttcctccgcgccaggttctcaagacattcaattgacgcagccgaatagcgtctctacgagcttggggagtaccgttaagctttcatgtactctttcctccggtaacatagagaacaactacgtgcattggtaccaactctacgagggcaggtctccaacgactatgatttacgatgacgataaacgacctgatggggtccctgaccgcttttcaggaagcattgacagaagctctaattccgcgtttttgactatccacaacgtagcgattgaagatgaagcaatttacttctgtcattcctatgtatccagctttaacgtcttcggaggtggaactaagttgacggtactccgagggggaggtgggtctcaccaccatcaccaccaccatcat

SEQ ID NO: 25 - DNA, 1743 bp, synthetic construct
  source 1..1743: mol_type = other DNA
aaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctccgccctgcgcgccctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcacggtggaagcagtagatcatccagttctggtgggggaggatccgggggcgggggtgaggttcagctccttgaatcaggcggagggctcgtccaaccaggcggatctttgcggctgtcatgtgccgcatccggattcacgttctcctcctttccgatggcatgggtgaggcaagctcctggaaaaggtctcgagtgggtatccaccataagtacttctggcgggcgcacttactacagggatagtgtgaagggaaggtttaccatttccagagataactcaaagaataccctgtacctgcaaatgaactccctcagggctgaagatacggctgtgtactactgtgctaaatttcgacaatactcaggtgggtttgactattggggccaggggacactggtgactgtgtcatcaagtcccaattccgcctcacatagcggaagtgccccacaaacttcctccgcgccaggttctcaagacattcaattgacgcagccgaatagcgtctctacgagcttggggagtaccgttaagctttcatgtactctttcctccggtaacatagagaacaactacgtgcattggtaccaactctacgagggcaggtctccaacgactatgatttacgatgacgataaacgacctgatggggtccctgaccgcttttcaggaagcattgacagaagctctaattccgcgtttttgactatccacaacgtagcgattgaagatgaagcaatttacttctgtcattcctatgtatccagctttaacgtcttcggaggtggaactaagttgacggtactccgagggggaggtgggtctcaccaccatcaccaccaccatcat

SEQ ID NO: 26 - DNA, 2538 bp, synthetic construct
  source 1..2538: mol_type = other DNA
cagatccagctggtgcagtccggcccagagctgaaaaagccaggcgagaccgtgaagatctcctgcaaggccagcggctacactttcaagaattatggcatgaactgggtgaagcaggcccccggcaaggggctgaagtggatggggtggattaacacctataccggccagcccatttacgccaacgatttcaaggggagattcgcctttagcctggaaaccagcgcgagcaccgcctatctgcagatcaacaatcttaagaatgaggatacagccacctacttttgcgcccgggattggggcccatactggggccagggcaccctggtgatcgtgtccgcttcccccaattctgcctcccacagcggcagcgccccacagactagctccgcccccggttcccagaatatcatgatgacacagagcccttccagcctggccgtgtctgccggcgaaaaggtgaccgtgaactgcaagtctagccagtctgtgctgtacagctccaatcagatgaactatctggcctggtaccagcagaagcctggccagagccctaagctgctgatttactgggccagcacacgcgagtctggggtccctgacagatttaccggtagcggaagcggcacagatttcacactgacaatctcttctgtgcagaccgaagacctcgcagtgtactactgtctgcagtatctgagctcttggactttcggcggggggaccaagctggaaattaagggcggatctagtcggtcatcctccagtggtggtgggggtagcgggggaggcggtaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctccgccctgcgcgccctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcacggtggaagcagtagatcatccagttctggtgggggaggatccgggggcgggggtgaggttcagctccttgaatcaggcggagggctcgtccaaccaggcggatctttgcggctgtcatgtgccgcatccggattcacgttctcctcctttccgatggcatgggtgaggcaagctcctggaaaaggtctcgagtgggtatccaccataagtacttctggcgggcgcacttactacagggatagtgtgaagggaaggtttaccatttccagagataactcaaagaataccctgtacctgcaaatgaactccctcagggctgaagatacggctgtgtactactgtgctaaatttcgacaatactcaggtgggtttgactattggggccaggggacactggtgactgtgtcatcaagtcccaattccgcctcacatagcggaagtgccccacaaacttcctccgcgccaggttctcaagacattcaattgacgcagccgaatagcgtctctacgagcttggggagtaccgttaagctttcatgtactctttcctccggtaacatagagaacaactacgtgcattggtaccaactctacgagggcaggtctccaacgactatgatttacgatgacgataaacgacctgatggggtccctgaccgcttttcaggaagcattgacagaagctctaattccgcgtttttgactatccacaacgtagcgattgaagatgaagcaatttacttctgtcattcctatgtatccagctttaacgtcttcggaggtggaactaagttgacggtactccgagggggaggtgggtctcaccaccatcaccaccaccatcat

SEQ ID NO: 27 - DNA, 1608 bp, synthetic construct
  source 1..1608: mol_type = other DNA
cagatccagctggtgcagtccggcccagagctgaaaaagccaggcgagaccgtgaagatctcctgcaaggccagcggctacactttcaagaattatggcatgaactgggtgaagcaggcccccggcaaggggctgaagtggatggggtggattaacacctataccggccagcccatttacgccaacgatttcaaggggagattcgcctttagcctggaaaccagcgcgagcaccgcctatctgcagatcaacaatcttaagaatgaggatacagccacctacttttgcgcccgggattggggcccatactggggccagggcaccctggtgatcgtgtccgcttcccccaattctgcctcccacagcggcagcgccccacagactagctccgcccccggttcccagaatatcatgatgacacagagcccttccagcctggccgtgtctgccggcgaaaaggtgaccgtgaactgcaagtctagccagtctgtgctgtacagctccaatcagatgaactatctggcctggtaccagcagaagcctggccagagccctaagctgctgatttactgggccagcacacgcgagtctggggtccctgacagatttaccggtagcggaagcggcacagatttcacactgacaatctcttctgtgcagaccgaagacctcgcagtgtactactgtctgcagtatctgagctcttggactttcggcggggggaccaagctggaaattaagggtggaagcagtagatcatccagtagtggggggggcggatctggaggtgggggagaggtgcggctgcagcagtccggccccgacctgatcaagcccggcgcctccgtgaagatgtcttgcaaagcctccggctacaccttcacaggatatgtgatgcactgggtgaagcagaggcccggccagggactggaatggatcggcttcatcaacccatacaacgatgacattcagtccaacgagagatttaggggaaaggccaccctgaccagtgacaaatcaagcactacagcctacatggagctgagctcactgacctctgaggactccgccgtgtattactgtgccagaggcgccggctacaattttgacggcgcctaccggttcttcgacttctgggggcagggcaccaccctgaccgtgtccagctcccccaatagtgcctcccattctggctccgccccccagactagctctgcccctgggtcccaggacgtggtgatgacccagtcccccctgtctctgcctgtgtccctgggagaccaggcctctatcagctgccggagctctcagaggctggtgcacagcaatgggaatacatacctgcactggtacctgcagaagccaggccagagccccaagctgctgatctacagagtgagcaacaggttccccggagtccctgataggtttagcggatctggcagcggcaccgacttcactctgaagattagcagagtggaggccgaggatctgggcatctacttttgctctcagtccacccatgtgccttacacctttgggggaggcaccaagctggagattaagaggggcgggggcggctcccaccaccaccatcaccaccaccac

SEQ ID NO: 28 - DNA, 1170 bp, synthetic construct
  source 1..1170: mol_type = other DNA
gctggacagccgctgcaagagcgggcacaagcctggggggaaagactcagggccagaatggaagaaatgggatcaagaacaagggacaggctcgatgaggtgaaggagcaagtagcagaggttcgagcaaagctggaagaacaagctcagcagattagattgcaagcagaggcggctcaagcacgactcaagagcaggttcgagccattggctgaagatatgcagcgacaatgggctggtcaggttgagaaagtccaagcggcggaagggaccagtgcagcaccggtaccatccgacaatcatggtggaagcagtagatcatccagtagtggggggggcggatctggaggtgggggagaggtgcggctgcagcagtccggccccgacctgatcaagcccggcgcctccgtgaagatgtcttgcaaagcctccggctacaccttcacaggatatgtgatgcactgggtgaagcagaggcccggccagggactggaatggatcggcttcatcaacccatacaacgatgacattcagtccaacgagagatttaggggaaaggccaccctgaccagtgacaaatcaagcactacagcctacatggagctgagctcactgacctctgaggactccgccgtgtattactgtgccagaggcgccggctacaattttgacggcgcctaccggttcttcgacttctgggggcagggcaccaccctgaccgtgtccagctcccccaatagtgcctcccattctggctccgccccccagactagctctgcccctgggtcccaggacgtggtgatgacccagtcccccctgtctctgcctgtgtccctgggagaccaggcctctatcagctgccggagctctcagaggctggtgcacagcaatgggaatacatacctgcactggtacctgcagaagccaggccagagccccaagctgctgatctacagagtgagcaacaggttccccggagtccctgataggtttagcggatctggcagcggcaccgacttcactctgaagattagcagagtggaggccgaggatctgggcatctacttttgctctcagtccacccatgtgccttacacctttgggggaggcaccaagctggagattaagaggggcgggggcggctcccaccaccaccatcaccaccaccac

SEQ ID NO: 29 - DNA, 1965 bp, synthetic construct
  source 1..1965: mol_type = other DNA
cagatccagctggtgcagtccggcccagagctgaaaaagccaggcgagaccgtgaagatctcctgcaaggccagcggctacactttcaagaattatggcatgaactgggtgaagcaggcccccggcaaggggctgaagtggatggggtggattaacacctataccggccagcccatttacgccaacgatttcaaggggagattcgcctttagcctggaaaccagcgcgagcaccgcctatctgcagatcaacaatcttaagaatgaggatacagccacctacttttgcgcccgggattggggcccatactggggccagggcaccctggtgatcgtgtccgcttcccccaattctgcctcccacagcggcagcgccccacagactagctccgcccccggttcccagaatatcatgatgacacagagcccttccagcctggccgtgtctgccggcgaaaaggtgaccgtgaactgcaagtctagccagtctgtgctgtacagctccaatcagatgaactatctggcctggtaccagcagaagcctggccagagccctaagctgctgatttactgggccagcacacgcgagtctggggtccctgacagatttaccggtagcggaagcggcacagatttcacactgacaatctcttctgtgcagaccgaagacctcgcagtgtactactgtctgcagtatctgagctcttggactttcggcggggggaccaagctggaaattaagggcggatctagtcggtcatcctccagtggtggtgggggtagcgggggaggcggtgctggacagccgctgcaagagcgggcacaagcctggggggaaagactcagggccagaatggaagaaatgggatcaagaacaagggacaggctcgatgaggtgaaggagcaagtagcagaggttcgagcaaagctggaagaacaagctcagcagattagattgcaagcagaggcggctcaagcacgactcaagagcaggttcgagccattggctgaagatatgcagcgacaatgggctggtcaggttgagaaagtccaagcggcggaagggaccagtgcagcaccggtaccatccgacaatcatggtggaagcagtagatcatccagtagtggggggggcggatctggaggtgggggagaggtgcggctgcagcagtccggccccgacctgatcaagcccggcgcctccgtgaagatgtcttgcaaagcctccggctacaccttcacaggatatgtgatgcactgggtgaagcagaggcccggccagggactggaatggatcggcttcatcaacccatacaacgatgacattcagtccaacgagagatttaggggaaaggccaccctgaccagtgacaaatcaagcactacagcctacatggagctgagctcactgacctctgaggactccgccgtgtattactgtgccagaggcgccggctacaattttgacggcgcctaccggttcttcgacttctgggggcagggcaccaccctgaccgtgtccagctcccccaatagtgcctcccattctggctccgccccccagactagctctgcccctgggtcccaggacgtggtgatgacccagtcccccctgtctctgcctgtgtccctgggagaccaggcctctatcagctgccggagctctcagaggctggtgcacagcaatgggaatacatacctgcactggtacctgcagaagccaggccagagccccaagctgctgatctacagagtgagcaacaggttccccggagtccctgataggtttagcggatctggcagcggcaccgacttcactctgaagattagcagagtggaggccgaggatctgggcatctacttttgctctcagtccacccatgtgccttacacctttgggggaggcaccaagctggagattaagaggggcgggggcggctcccaccaccaccatcaccaccaccac

SEQ ID NO: 30 - DNA, 1764 bp, synthetic construct
  source 1..1764: mol_type = other DNA
aaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctccgccctgcgcgccctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcacggtggaagcagtagatcatccagttctggtgggggaggatccgggggcgggggtgaggtgcggctgcagcagtccggccccgacctgatcaagcccggcgcctccgtgaagatgtcttgcaaagcctccggctacaccttcacaggatatgtgatgcactgggtgaagcagaggcccggccagggactggaatggatcggcttcatcaacccatacaacgatgacattcagtccaacgagagatttaggggaaaggccaccctgaccagtgacaaatcaagcactacagcctacatggagctgagctcactgacctctgaggactccgccgtgtattactgtgccagaggcgccggctacaattttgacggcgcctaccggttcttcgacttctgggggcagggcaccaccctgaccgtgtccagctcccccaatagtgcctcccattctggctccgccccccagactagctctgcccctgggtcccaggacgtggtgatgacccagtcccccctgtctctgcctgtgtccctgggagaccaggcctctatcagctgccggagctctcagaggctggtgcacagcaatgggaatacatacctgcactggtacctgcagaagccaggccagagccccaagctgctgatctacagagtgagcaacaggttccccggagtccctgataggtttagcggatctggcagcggcaccgacttcactctgaagattagcagagtggaggccgaggatctgggcatctacttttgctctcagtccacccatgtgccttacacctttgggggaggcaccaagctggagattaagaggggcgggggcggctcccaccaccaccatcaccaccaccac

SEQ ID NO: 31 - DNA, 2559 bp, synthetic construct
  source 1..2559: mol_type = other DNA
cagatccagctggtgcagtccggcccagagctgaaaaagccaggcgagaccgtgaagatctcctgcaaggccagcggctacactttcaagaattatggcatgaactgggtgaagcaggcccccggcaaggggctgaagtggatggggtggattaacacctataccggccagcccatttacgccaacgatttcaaggggagattcgcctttagcctggaaaccagcgcgagcaccgcctatctgcagatcaacaatcttaagaatgaggatacagccacctacttttgcgcccgggattggggcccatactggggccagggcaccctggtgatcgtgtccgcttcccccaattctgcctcccacagcggcagcgccccacagactagctccgcccccggttcccagaatatcatgatgacacagagcccttccagcctggccgtgtctgccggcgaaaaggtgaccgtgaactgcaagtctagccagtctgtgctgtacagctccaatcagatgaactatctggcctggtaccagcagaagcctggccagagccctaagctgctgatttactgggccagcacacgcgagtctggggtccctgacagatttaccggtagcggaagcggcacagatttcacactgacaatctcttctgtgcagaccgaagacctcgcagtgtactactgtctgcagtatctgagctcttggactttcggcggggggaccaagctggaaattaagggcggatctagtcggtcatcctccagtggtggtgggggtagcgggggaggcggtaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctccgccctgcgcgccctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcacggtggaagcagtagatcatccagttctggtgggggaggatccgggggcgggggtgaggtgcggctgcagcagtccggccccgacctgatcaagcccggcgcctccgtgaagatgtcttgcaaagcctccggctacaccttcacaggatatgtgatgcactgggtgaagcagaggcccggccagggactggaatggatcggcttcatcaacccatacaacgatgacattcagtccaacgagagatttaggggaaaggccaccctgaccagtgacaaatcaagcactacagcctacatggagctgagctcactgacctctgaggactccgccgtgtattactgtgccagaggcgccggctacaattttgacggcgcctaccggttcttcgacttctgggggcagggcaccaccctgaccgtgtccagctcccccaatagtgcctcccattctggctccgccccccagactagctctgcccctgggtcccaggacgtggtgatgacccagtcccccctgtctctgcctgtgtccctgggagaccaggcctctatcagctgccggagctctcagaggctggtgcacagcaatgggaatacatacctgcactggtacctgcagaagccaggccagagccccaagctgctgatctacagagtgagcaacaggttccccggagtccctgataggtttagcggatctggcagcggcaccgacttcactctgaagattagcagagtggaggccgaggatctgggcatctacttttgctctcagtccacccatgtgccttacacctttgggggaggcaccaagctggagattaagaggggcgggggcggctcccaccaccaccatcaccaccaccac

SEQ ID NO: 32 - DNA, 1572 bp, synthetic construct
  source 1..1572: mol_type = other DNA
cagatccagctggtgcagtccggcccagagctgaaaaagccaggcgagaccgtgaagatctcctgcaaggccagcggctacactttcaagaattatggcatgaactgggtgaagcaggcccccggcaaggggctgaagtggatggggtggattaacacctataccggccagcccatttacgccaacgatttcaaggggagattcgcctttagcctggaaaccagcgcgagcaccgcctatctgcagatcaacaatcttaagaatgaggatacagccacctacttttgcgcccgggattggggcccatactggggccagggcaccctggtgatcgtgtccgcttcccccaattctgcctcccacagcggcagcgccccacagactagctccgcccccggttcccagaatatcatgatgacacagagcccttccagcctggccgtgtctgccggcgaaaaggtgaccgtgaactgcaagtctagccagtctgtgctgtacagctccaatcagatgaactatctggcctggtaccagcagaagcctggccagagccctaagctgctgatttactgggccagcacacgcgagtctggggtccctgacagatttaccggtagcggaagcggcacagatttcacactgacaatctcttctgtgcagaccgaagacctcgcagtgtactactgtctgcagtatctgagctcttggactttcggcggggggaccaagctggaaattaagggtggaagcagtagatcatccagttctggtgggggaggatccgggggcgggggtcagatccagctcgtacaaagtggtccaggtctcaaaaaaccaggaggaagcgtccggataagctgcgccgcttcaggatatacctttacgaattacggtatgaattgggtaaaacaggctccgggaaaaggactcagatggatggggtggatcaatacgcacacgggcgagccgacttacgcggatgattttaagggtcgctttactttttccctcgacacctcaaaatccactgcctatctgcaaataaacagtcttcgcgcagaggatacagccacctatttttgcacaaggcgggggtacgattggtatttcgatgtatgggggcagggtacaacggtaacagtgagttcaagtcccaacagcgcctctcacagcggcagcgcacctcagacgagctctgctcctggcagccaagacatccagatgacccagagcccttcctctatgtccgcatccctcggagaccgcgtaacaatcacatgtcgagccagtcaagacatcaatagctatcttagttggttccagcagaaacctggaaaaagtcctaagacccttatttatagagcgaataggttggttgatggagtcccgtctaggtttagcgggtctgggtctgggactgactacacccttactatctcctctctgcaatacgaagacttcggcatttattattgccagcaatatgatgagtctccctggactttcggtggggggaccaagttggaaataaaaggcgggggcggctcccaccaccaccatcaccaccaccac

SEQ ID NO: 33 - DNA, 1134 bp, synthetic construct
  source 1..1134: mol_type = other DNA
gctggacagccgctgcaagagcgggcacaagcctggggggaaagactcagggccagaatggaagaaatgggatcaagaacaagggacaggctcgatgaggtgaaggagcaagtagcagaggttcgagcaaagctggaagaacaagctcagcagattagattgcaagcagaggcggctcaagcacgactcaagagcaggttcgagccattggctgaagatatgcagcgacaatgggctggtcaggttgagaaagtccaagcggcggaagggaccagtgcagcaccggtaccatccgacaatcatggtggaagcagtagatcatccagttctggtgggggaggatccgggggcgggggtcagatccagctcgtacaaagtggtccaggtctcaaaaaaccaggaggaagcgtccggataagctgcgccgcttcaggatatacctttacgaattacggtatgaattgggtaaaacaggctccgggaaaaggactcagatggatggggtggatcaatacgcacacgggcgagccgacttacgcggatgattttaagggtcgctttactttttccctcgacacctcaaaatccactgcctatctgcaaataaacagtcttcgcgcagaggatacagccacctatttttgcacaaggcgggggtacgattggtatttcgatgtatgggggcagggtacaacggtaacagtgagttcaagtcccaacagcgcctctcacagcggcagcgcacctcagacgagctctgctcctggcagccaagacatccagatgacccagagcccttcctctatgtccgcatccctcggagaccgcgtaacaatcacatgtcgagccagtcaagacatcaatagctatcttagttggttccagcagaaacctggaaaaagtcctaagacccttatttatagagcgaataggttggttgatggagtcccgtctaggtttagcgggtctgggtctgggactgactacacccttactatctcctctctgcaatacgaagacttcggcatttattattgccagcaatatgatgagtctccctggactttcggtggggggaccaagttggaaataaaaggcgggggcggctcccaccaccaccatcaccaccaccac

SEQ ID NO: 34 - DNA, 1929 bp, synthetic construct
  source 1..1929: mol_type = other DNA
cagatccagctggtgcagtccggcccagagctgaaaaagccaggcgagaccgtgaagatctcctgcaaggccagcggctacactttcaagaattatggcatgaactgggtgaagcaggcccccggcaaggggctgaagtggatggggtggattaacacctataccggccagcccatttacgccaacgatttcaaggggagattcgcctttagcctggaaaccagcgcgagcaccgcctatctgcagatcaacaatcttaagaatgaggatacagccacctacttttgcgcccgggattggggcccatactggggccagggcaccctggtgatcgtgtccgcttcccccaattctgcctcccacagcggcagcgccccacagactagctccgcccccggttcccagaatatcatgatgacacagagcccttccagcctggccgtgtctgccggcgaaaaggtgaccgtgaactgcaagtctagccagtctgtgctgtacagctccaatcagatgaactatctggcctggtaccagcagaagcctggccagagccctaagctgctgatttactgggccagcacacgcgagtctggggtccctgacagatttaccggtagcggaagcggcacagatttcacactgacaatctcttctgtgcagaccgaagacctcgcagtgtactactgtctgcagtatctgagctcttggactttcggcggggggaccaagctggaaattaagggcggatctagtcggtcatcctccagtggtggtgggggtagcgggggaggcggtgctggacagccgctgcaagagcgggcacaagcctggggggaaagactcagggccagaatggaagaaatgggatcaagaacaagggacaggctcgatgaggtgaaggagcaagtagcagaggttcgagcaaagctggaagaacaagctcagcagattagattgcaagcagaggcggctcaagcacgactcaagagcaggttcgagccattggctgaagatatgcagcgacaatgggctggtcaggttgagaaagtccaagcggcggaagggaccagtgcagcaccggtaccatccgacaatcatggtggaagcagtagatcatccagttctggtgggggaggatccgggggcgggggtcagatccagctcgtacaaagtggtccaggtctcaaaaaaccaggaggaagcgtccggataagctgcgccgcttcaggatatacctttacgaattacggtatgaattgggtaaaacaggctccgggaaaaggactcagatggatggggtggatcaatacgcacacgggcgagccgacttacgcggatgattttaagggtcgctttactttttccctcgacacctcaaaatccactgcctatctgcaaataaacagtcttcgcgcagaggatacagccacctatttttgcacaaggcgggggtacgattggtatttcgatgtatgggggcagggtacaacggtaacagtgagttcaagtcccaacagcgcctctcacagcggcagcgcacctcagacgagctctgctcctggcagccaagacatccagatgacccagagcccttcctctatgtccgcatccctcggagaccgcgtaacaatcacatgtcgagccagtcaagacatcaatagctatcttagttggttccagcagaaacctggaaaaagtcctaagacccttatttatagagcgaataggttggttgatggagtcccgtctaggtttagcgggtctgggtctgggactgactacacccttactatctcctctctgcaatacgaagacttcggcatttattattgccagcaatatgatgagtctccctggactttcggtggggggaccaagttggaaataaaaggcgggggcggctcccaccaccaccatcaccaccaccac

SEQ ID NO: 35 - DNA, 1728 bp, synthetic construct
  source 1..1728: mol_type = other DNA
aaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctccgccctgcgcgccctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcacggtggaagcagtagatcatccagttctggtgggggaggatccgggggcgggggtcagatccagctcgtacaaagtggtccaggtctcaaaaaaccaggaggaagcgtccggataagctgcgccgcttcaggatatacctttacgaattacggtatgaattgggtaaaacaggctccgggaaaaggactcagatggatggggtggatcaatacgcacacgggcgagccgacttacgcggatgattttaagggtcgctttactttttccctcgacacctcaaaatccactgcctatctgcaaataaacagtcttcgcgcagaggatacagccacctatttttgcacaaggcgggggtacgattggtatttcgatgtatgggggcagggtacaacggtaacagtgagttcaagtcccaacagcgcctctcacagcggcagcgcacctcagacgagctctgctcctggcagccaagacatccagatgacccagagcccttcctctatgtccgcatccctcggagaccgcgtaacaatcacatgtcgagccagtcaagacatcaatagctatcttagttggttccagcagaaacctggaaaaagtcctaagacccttatttatagagcgaataggttggttgatggagtcccgtctaggtttagcgggtctgggtctgggactgactacacccttactatctcctctctgcaatacgaagacttcggcatttattattgccagcaatatgatgagtctccctggactttcggtggggggaccaagttggaaataaaaggcgggggcggctcccaccaccaccatcaccaccaccac

SEQ ID NO: 36 - DNA, 2523 bp, synthetic construct
  source 1..2523: mol_type = other DNA
cagatccagctggtgcagtccggcccagagctgaaaaagccaggcgagaccgtgaagatctcctgcaaggccagcggctacactttcaagaattatggcatgaactgggtgaagcaggcccccggcaaggggctgaagtggatggggtggattaacacctataccggccagcccatttacgccaacgatttcaaggggagattcgcctttagcctggaaaccagcgcgagcaccgcctatctgcagatcaacaatcttaagaatgaggatacagccacctacttttgcgcccgggattggggcccatactggggccagggcaccctggtgatcgtgtccgcttcccccaattctgcctcccacagcggcagcgccccacagactagctccgcccccggttcccagaatatcatgatgacacagagcccttccagcctggccgtgtctgccggcgaaaaggtgaccgtgaactgcaagtctagccagtctgtgctgtacagctccaatcagatgaactatctggcctggtaccagcagaagcctggccagagccctaagctgctgatttactgggccagcacacgcgagtctggggtccctgacagatttaccggtagcggaagcggcacagatttcacactgacaatctcttctgtgcagaccgaagacctcgcagtgtactactgtctgcagtatctgagctcttggactttcggcggggggaccaagctggaaattaagggcggatctagtcggtcatcctccagtggtggtgggggtagcgggggaggcggtaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctccgccctgcgcgccctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcacggtggaagcagtagatcatccagttctggtgggggaggatccgggggcgggggtcagatccagctcgtacaaagtggtccaggtctcaaaaaaccaggaggaagcgtccggataagctgcgccgcttcaggatatacctttacgaattacggtatgaattgggtaaaacaggctccgggaaaaggactcagatggatggggtggatcaatacgcacacgggcgagccgacttacgcggatgattttaagggtcgctttactttttccctcgacacctcaaaatccactgcctatctgcaaataaacagtcttcgcgcagaggatacagccacctatttttgcacaaggcgggggtacgattggtatttcgatgtatgggggcagggtacaacggtaacagtgagttcaagtcccaacagcgcctctcacagcggcagcgcacctcagacgagctctgctcctggcagccaagacatccagatgacccagagcccttcctctatgtccgcatccctcggagaccgcgtaacaatcacatgtcgagccagtcaagacatcaatagctatcttagttggttccagcagaaacctggaaaaagtcctaagacccttatttatagagcgaataggttggttgatggagtcccgtctaggtttagcgggtctgggtctgggactgactacacccttactatctcctctctgcaatacgaagacttcggcatttattattgccagcaatatgatgagtctccctggactttcggtggggggaccaagttggaaataaaaggcgggggcggctcccaccaccaccatcaccaccaccac

SEQ ID NO: 37 - DNA, 1581 bp, synthetic construct
  source 1..1581: mol_type = other DNA
cagatccagctggtgcagtccggcccagagctgaaaaagccaggcgagaccgtgaagatctcctgcaaggccagcggctacactttcaagaattatggcatgaactgggtgaagcaggcccccggcaaggggctgaagtggatggggtggattaacacctataccggccagcccatttacgccaacgatttcaaggggagattcgcctttagcctggaaaccagcgcgagcaccgcctatctgcagatcaacaatcttaagaatgaggatacagccacctacttttgcgcccgggattggggcccatactggggccagggcaccctggtgatcgtgtccgcttcccccaattctgcctcccacagcggcagcgccccacagactagctccgcccccggttcccagaatatcatgatgacacagagcccttccagcctggccgtgtctgccggcgaaaaggtgaccgtgaactgcaagtctagccagtctgtgctgtacagctccaatcagatgaactatctggcctggtaccagcagaagcctggccagagccctaagctgctgatttactgggccagcacacgcgagtctggggtccctgacagatttaccggtagcggaagcggcacagatttcacactgacaatctcttctgtgcagaccgaagacctcgcagtgtactactgtctgcagtatctgagctcttggactttcggcggggggaccaagctggaaattaagggtggaagcagtagatcatccagtagtggggggggcggatctggaggtgggggacaggtgaaactgcagcagtctggccccgagctgaagaagcccggcgagaccgtgaagatctcctgcaaggcctccggctacacctttaccaattacggaatgaactgggtgaagcaggctccaggaaagggcctgaaatggatgggctggatcaacacttatactggcgagagcacctacgcagacgatttcaagggtaggttcgcatttagcctggagacctccgccagcgccgcctacctgcagatcaataatctgaaaaacgaggacacagccacctatttctgcgcccggtttgccatcaaaggggattactggggccagggcaccacagtgaccgtgtcctcctcccccaatagtgcctcccattctggctccgccccccagactagctctgcccctgggtcccaggatattgtcctgacccagtccccattctccaaccccgtgaccctggggaccagcgcctccattagctgtaggtcaactaagagcctgctgcacagcaatggaatcacctacctgtactggtacctgcagaagccaggccagtcccctcagctgctgatctaccagatgagcaacctggcctcaggcgtgcctgatcggttttccagctccggcagcggcaccgacttcaccctgcggatctccagggtggaggctgaagacgtgggggtgtactactgcgcccagaacctggagatccctaggacatttggaggcggcaccaagctggagatcaagggcgggggcggctcccaccaccaccatcaccaccaccac

SEQ ID NO: 38 - protein, 247 aa, synthetic construct
QIQLVQSGPELKKPGETVKISCKASGYTFKNYGMNWVKQAPGKGLKWMGWINTYTGQPIYANDFKGRFAFSLETSASTAYLQINNLKNEDTATYFCARDWGPYWGQGTLVIVSASPNSASHSGSAPQTSSAPGSQNIMMTQSPSSLAVSAGEKVTVNCKSSQSVLYSSNQMNYLAWYQQKPGQSPKLLIYWASTRESGVPDRFTGSGSGTDFTLTISSVQTEDLAVYYCLQYLSSWTFGGGTKLEIK

SEQ ID NO: 39 - protein, 251 aa, synthetic construct
EVQLLESGGGLVQPGGSLRLSCAASGFTFSSFPMAWVRQAPGKGLEWVSTISTSGGRTYYRDSVKGRFTISRDNSKNTLYLQMNSLRAEDTAVYYCAKFRQYSGGFDYWGQGTLVTVSSSPNSASHSGSAPQTSSAPGSQDIQLTQPNSVSTSLGSTVKLSCTLSSGNIENNYVHWYQLYEGRSPTTMIYDDDKRPDGVPDRFSGSIDRSSNSAFLTIHNVAIEDEAIYFCHSYVSSFNVFGGGTKLTVLR

SEQ ID NO: 40 - protein, 101 aa, synthetic construct
AGQPLQERAQAWGERLRARMEEMGSRTRDRLDEVKEQVAEVRAKLEEQAQQIRLQAEAAQARLKSRFEPLAEDMQRQWAGQVEKVQAAEGTSAAPVPSDNH

SEQ ID NO: 41 - protein, 299 aa, synthetic construct
KVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASALRALRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 42 - protein, 257 aa, synthetic construct
EVRLQQSGPDLIKPGASVKMSCKASGYTFTGYVMHWVKQRPGQGLEWIGFINPYNDDIQSNERFRGKATLTSDKSSTTAYMELSSLTSEDSAVYYCARGAGYNFDGAYRFFDFWGQGTTLTVSSSPNSASHSGSAPQTSSAPGSQDVVMTQSPLSLPVSLGDQASISCRSSQRLVHSNGNTYLHWYLQKPGQSPKLLIYRVSNRFPGVPDRFSGSGSGTDFTLKISRVEAEDLGIYFCSQSTHVPYTFGGGTKLEIK

SEQ ID NO: 43 - protein, 246 aa, synthetic construct
QIQLVQSGPGLKKPGGSVRISCAASGYTFTNYGMNWVKQAPGKGLRWMGWINTHTGEPTYADDFKGRFTFSLDTSKSTAYLQINSLRAEDTATYFCTRRGYDWYFDVWGQGTTVTVSSSPNSASHSGSAPQTSSAPGSQDIQMTQSPSSMSASLGDRVTITCRASQDINSYLSWFQQKPGKSPKTLIYRANRLVDGVPSRFSGSGSGTDYTLTISSLQYEDFGIYYCQQYDESPWTFGGGTKLEIK

SEQ ID NO: 44 - protein, 249 aa, synthetic construct
QVKLQQSGPELKKPGETVKISCKASGYTFTNYGMNWVKQAPGKGLKWMGWINTYTGESTYADDFKGRFAFSLETSASAAYLQINNLKNEDTATYFCARFAIKGDYWGQGTTVTVSSSPNSASHSGSAPQTSSAPGSQDIVLTQSPFSNPVTLGTSASISCRSTKSLLHSNGITYLYWYLQKPGQSPQLLIYQMSNLASGVPDRFSSSGSGTDFTLRISRVEAEDVGVYYCAQNLEIPRTFGGGTKLEIK

SEQ ID NO: 45 - DNA, 741 bp, synthetic construct
  source 1..741: mol_type = other DNA
cagatccagctggtgcagtccggcccagagctgaaaaagccaggcgagaccgtgaagatctcctgcaaggccagcggctacactttcaagaattatggcatgaactgggtgaagcaggcccccggcaaggggctgaagtggatggggtggattaacacctataccggccagcccatttacgccaacgatttcaaggggagattcgcctttagcctggaaaccagcgcgagcaccgcctatctgcagatcaacaatcttaagaatgaggatacagccacctacttttgcgcccgggattggggcccatactggggccagggcaccctggtgatcgtgtccgcttcccccaattctgcctcccacagcggcagcgccccacagactagctccgcccccggttcccagaatatcatgatgacacagagcccttccagcctggccgtgtctgccggcgaaaaggtgaccgtgaactgcaagtctagccagtctgtgctgtacagctccaatcagatgaactatctggcctggtaccagcagaagcctggccagagccctaagctgctgatttactgggccagcacacgcgagtctggggtccctgacagatttaccggtagcggaagcggcacagatttcacactgacaatctcttctgtgcagaccgaagacctcgcagtgtactactgtctgcagtatctgagctcttggactttcggcggggggaccaagctggaaattaag

SEQ ID NO: 46 - DNA, 54 bp, synthetic construct
  source 1..54: mol_type = other DNA
ggtggaagcagtagatcatccagttctggtgggggaggatccgggggcgggggt

SEQ ID NO: 47 - DNA, 753 bp, synthetic construct
  source 1..753: mol_type = other DNA
gaggttcagctccttgaatcaggcggagggctcgtccaaccaggcggatctttgcggctgtcatgtgccgcatccggattcacgttctcctcctttccgatggcatgggtgaggcaagctcctggaaaaggtctcgagtgggtatccaccataagtacttctggcgggcgcacttactacagggatagtgtgaagggaaggtttaccatttccagagataactcaaagaataccctgtacctgcaaatgaactccctcagggctgaagatacggctgtgtactactgtgctaaatttcgacaatactcaggtgggtttgactattggggccaggggacactggtgactgtgtcatcaagtcccaattccgcctcacatagcggaagtgccccacaaacttcctccgcgccaggttctcaagacattcaattgacgcagccgaatagcgtctctacgagcttggggagtaccgttaagctttcatgtactctttcctccggtaacatagagaacaactacgtgcattggtaccaactctacgagggcaggtctccaacgactatgatttacgatgacgataaacgacctgatggggtccctgaccgcttttcaggaagcattgacagaagctctaattccgcgtttttgactatccacaacgtagcgattgaagatgaagcaatttacttctgtcattcctatgtatccagctttaacgtcttcggaggtggaactaagttgacggtactccga

SEQ ID NO: 48 - DNA, 303 bp, synthetic construct
  source 1..303: mol_type = other DNA
gctggacagccgctgcaagagcgggcacaagcctggggggaaagactcagggccagaatggaagaaatgggatcaagaacaagggacaggctcgatgaggtgaaggagcaagtagcagaggttcgagcaaagctggaagaacaagctcagcagattagattgcaagcagaggcggctcaagcacgactcaagagcaggttcgagccattggctgaagatatgcagcgacaatgggctggtcaggttgagaaagtccaagcggcggaagggaccagtgcagcaccggtaccatccgacaatcat

SEQ ID NO: 49 - DNA, 897 bp, synthetic construct
  source 1..897: mol_type = other DNA
aaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctccgccctgcgcgccctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcac

SEQ ID NO: 50 - DNA, 774 bp, synthetic construct
  source 1..774: mol_type = other DNA
gaggtgcggctgcagcagtccggccccgacctgatcaagcccggcgcctccgtgaagatgtcttgcaaagcctccggctacaccttcacaggatatgtgatgcactgggtgaagcagaggcccggccagggactggaatggatcggcttcatcaacccatacaacgatgacattcagtccaacgagagatttaggggaaaggccaccctgaccagtgacaaatcaagcactacagcctacatggagctgagctcactgacctctgaggactccgccgtgtattactgtgccagaggcgccggctacaattttgacggcgcctaccggttcttcgacttctgggggcagggcaccaccctgaccgtgtccagctcccccaatagtgcctcccattctggctccgccccccagactagctctgcccctgggtcccaggacgtggtgatgacccagtcccccctgtctctgcctgtgtccctgggagaccaggcctctatcagctgccggagctctcagaggctggtgcacagcaatgggaatacatacctgcactggtacctgcagaagccaggccagagccccaagctgctgatctacagagtgagcaacaggttccccggagtccctgataggtttagcggatctggcagcggcaccgacttcactctgaagattagcagagtggaggccgaggatctgggcatctacttttgctctcagtccacccatgtgccttacacctttgggggaggcaccaagctggagattaagagg

SEQ ID NO: 51 - DNA, 738 bp, synthetic construct
  source 1..738: mol_type = other DNA
cagatccagctcgtacaaagtggtccaggtctcaaaaaaccaggaggaagcgtccggataagctgcgccgcttcaggatatacctttacgaattacggtatgaattgggtaaaacaggctccgggaaaaggactcagatggatggggtggatcaatacgcacacgggcgagccgacttacgcggatgattttaagggtcgctttactttttccctcgacacctcaaaatccactgcctatctgcaaataaacagtcttcgcgcagaggatacagccacctatttttgcacaaggcgggggtacgattggtatttcgatgtatgggggcagggtacaacggtaacagtgagttcaagtcccaacagcgcctctcacagcggcagcgcacctcagacgagctctgctcctggcagccaagacatccagatgacccagagcccttcctctatgtccgcatccctcggagaccgcgtaacaatcacatgtcgagccagtcaagacatcaatagctatcttagttggttccagcagaaacctggaaaaagtcctaagacccttatttatagagcgaataggttggttgatggagtcccgtctaggtttagcgggtctgggtctgggactgactacacccttactatctcctctctgcaatacgaagacttcggcatttattattgccagcaatatgatgagtctccctggactttcggtggggggaccaagttggaaataaaa

SEQ ID NO: 52 - DNA, 747 bp, synthetic construct
  source 1..747: mol_type = other DNA
caggtgaaactgcagcagtctggccccgagctgaagaagcccggcgagaccgtgaagatctcctgcaaggcctccggctacacctttaccaattacggaatgaactgggtgaagcaggctccaggaaagggcctgaaatggatgggctggatcaacacttatactggcgagagcacctacgcagacgatttcaagggtaggttcgcatttagcctggagacctccgccagcgccgcctacctgcagatcaataatctgaaaaacgaggacacagccacctatttctgcgcccggtttgccatcaaaggggattactggggccagggcaccacagtgaccgtgtcctcctcccccaatagtgcctcccattctggctccgccccccagactagctctgcccctgggtcccaggatattgtcctgacccagtccccattctccaaccccgtgaccctggggaccagcgcctccattagctgtaggtcaactaagagcctgctgcacagcaatggaatcacctacctgtactggtacctgcagaagccaggccagtcccctcagctgctgatctaccagatgagcaacctggcctcaggcgtgcctgatcggttttccagctccggcagcggcaccgacttcaccctgcggatctccagggtggaggctgaagacgtgggggtgtactactgcgcccagaacctggagatccctaggacatttggaggcggcaccaagctggagatcaag

SEQ ID NO: 53 - protein, 20 aa, synthetic construct
MYRMQLLSCIALSLALVTNS

SEQ ID NO: 54 - DNA, 60 bp, synthetic construct
  source 1..60: mol_type = other DNA
atgtacaggatgcaactcctgtcttgcattgcactaagtcttgcacttgtcacgaattcg

SEQ ID NO: 55 - protein, 549 aa, synthetic construct
MYRMQLLSCIALSLALVTNSQIQLVQSGPELKKPGETVKISCKASGYTFKNYGMNWVKQAPGKGLKWMGWINTYTGQPIYANDFKGRFAFSLETSASTAYLQINNLKNEDTATYFCARDWGPYWGQGTLVIVSASPNSASHSGSAPQTSSAPGSQNIMMTQSPSSLAVSAGEKVTVNCKSSQSVLYSSNQMNYLAWYQQKPGQSPKLLIYWASTRESGVPDRFTGSGSGTDFTLTISSVQTEDLAVYYCLQYLSSWTFGGGTKLEIKGGSSRSSSSGGGGSGGGGEVQLLESGGGLVQPGGSLRLSCAASGFTFSSFPMAWVRQAPGKGLEWVSTISTSGGRTYYRDSVKGRFTISRDNSKNTLYLQMNSLRAEDTAVYYCAKFRQYSGGFDYWGQGTLVTVSSSPNSASHSGSAPQTSSAPGSQDIQLTQPNSVSTSLGSTVKLSCTLSSGNIENNYVHWYQLYEGRSPTTMIYDDDKRPDGVPDRFSGSIDRSSNSAFLTIHNVAIEDEAIYFCHSYVSSFNVFGGGTKLTVLRGGGGSHHHHHHHH

SEQ ID NO: 56 - protein, 403 aa, synthetic construct
MYRMQLLSCIALSLALVTNSAGQPLQERAQAWGERLRARMEEMGSRTRDRLDEVKEQVAEVRAKLEEQAQQIRLQAEAAQARLKSRFEPLAEDMQRQWAGQVEKVQAAEGTSAAPVPSDNHGGSSRSSSSGGGGSGGGGEVQLLESGGGLVQPGGSLRLSCAASGFTFSSFPMAWVRQAPGKGLEWVSTISTSGGRTYYRDSVKGRFTISRDNSKNTLYLQMNSLRAEDTAVYYCAKFRQYSGGFDYWGQGTLVTVSSSPNSASHSGSAPQTSSAPGSQDIQLTQPNSVSTSLGSTVKLSCTLSSGNIENNYVHWYQLYEGRSPTTMIYDDDKRPDGVPDRFSGSIDRSSNSAFLTIHNVAIEDEAIYFCHSYVSSFNVFGGGTKLTVLRGGGGSHHHHHHHH

SEQ ID NO: 57 - protein, 668 aa, synthetic construct
MYRMQLLSCIALSLALVTNSQIQLVQSGPELKKPGETVKISCKASGYTFKNYGMNWVKQAPGKGLKWMGWINTYTGQPIYANDFKGRFAFSLETSASTAYLQINNLKNEDTATYFCARDWGPYWGQGTLVIVSASPNSASHSGSAPQTSSAPGSQNIMMTQSPSSLAVSAGEKVTVNCKSSQSVLYSSNQMNYLAWYQQKPGQSPKLLIYWASTRESGVPDRFTGSGSGTDFTLTISSVQTEDLAVYYCLQYLSSWTFGGGTKLEIKGGSSRSSSSGGGGSGGGGAGQPLQERAQAWGERLRARMEEMGSRTRDRLDEVKEQVAEVRAKLEEQAQQIRLQAEAAQARLKSRFEPLAEDMQRQWAGQVEKVQAAEGTSAAPVPSDNHGGSSRSSSSGGGGSGGGGEVQLLESGGGLVQPGGSLRLSCAASGFTFSSFPMAWVRQAPGKGLEWVSTISTSGGRTYYRDSVKGRFTISRDNSKNTLYLQMNSLRAEDTAVYYCAKFRQYSGGFDYWGQGTLVTVSSSPNSASHSGSAPQTSSAPGSQDIQLTQPNSVSTSLGSTVKLSCTLSSGNIENNYVHWYQLYEGRSPTTMIYDDDKRPDGVPDRFSGSIDRSSNSAFLTIHNVAIEDEAIYFCHSYVSSFNVFGGGTKLTVLRGGGGSHHHHHHHH

SEQ ID NO: 58 - DNA, 1647 bp, synthetic construct
  source 1..1647: mol_type = other DNA
atgtacaggatgcaactcctgtcttgcattgcactaagtcttgcacttgtcacgaattcgcagatccagctggtgcagtccggcccagagctgaaaaagccaggcgagaccgtgaagatctcctgcaaggccagcggctacactttcaagaattatggcatgaactgggtgaagcaggcccccggcaaggggctgaagtggatggggtggattaacacctataccggccagcccatttacgccaacgatttcaaggggagattcgcctttagcctggaaaccagcgcgagcaccgcctatctgcagatcaacaatcttaagaatgaggatacagccacctacttttgcgcccgggattggggcccatactggggccagggcaccctggtgatcgtgtccgcttcccccaattctgcctcccacagcggcagcgccccacagactagctccgcccccggttcccagaatatcatgatgacacagagcccttccagcctggccgtgtctgccggcgaaaaggtgaccgtgaactgcaagtctagccagtctgtgctgtacagctccaatcagatgaactatctggcctggtaccagcagaagcctggccagagccctaagctgctgatttactgggccagcacacgcgagtctggggtccctgacagatttaccggtagcggaagcggcacagatttcacactgacaatctcttctgtgcagaccgaagacctcgcagtgtactactgtctgcagtatctgagctcttggactttcggcggggggaccaagctggaaattaagggtggaagcagtagatcatccagttctggtgggggaggatccgggggcgggggtgaggttcagctccttgaatcaggcggagggctcgtccaaccaggcggatctttgcggctgtcatgtgccgcatccggattcacgttctcctcctttccgatggcatgggtgaggcaagctcctggaaaaggtctcgagtgggtatccaccataagtacttctggcgggcgcacttactacagggatagtgtgaagggaaggtttaccatttccagagataactcaaagaataccctgtacctgcaaatgaactccctcagggctgaagatacggctgtgtactactgtgctaaatttcgacaatactcaggtgggtttgactattggggccaggggacactggtgactgtgtcatcaagtcccaattccgcctcacatagcggaagtgccccacaaacttcctccgcgccaggttctcaagacattcaattgacgcagccgaatagcgtctctacgagcttggggagtaccgttaagctttcatgtactctttcctccggtaacatagagaacaactacgtgcattggtaccaactctacgagggcaggtctccaacgactatgatttacgatgacgataaacgacctgatggggtccctgaccgcttttcaggaagcattgacagaagctctaattccgcgtttttgactatccacaacgtagcgattgaagatgaagcaatttacttctgtcattcctatgtatccagctttaacgtcttcggaggtggaactaagttgacggtactccgagggggaggtgggtctcaccaccatcaccaccaccatcat

SEQ ID NO: 59 - DNA, 1209 bp, synthetic construct
  source 1..1209: mol_type = other DNA
atgtacaggatgcaactcctgtcttgcattgcactaagtcttgcacttgtcacgaattcggctggacagccgctgcaagagcgggcacaagcctggggggaaagactcagggccagaatggaagaaatgggatcaagaacaagggacaggctcgatgaggtgaaggagcaagtagcagaggttcgagcaaagctggaagaacaagctcagcagattagattgcaagcagaggcggctcaagcacgactcaagagcaggttcgagccattggctgaagatatgcagcgacaatgggctggtcaggttgagaaagtccaagcggcggaagggaccagtgcagcaccggtaccatccgacaatcatggtggaagcagtagatcatccagttctggtgggggaggatccgggggcgggggtgaggttcagctccttgaatcaggcggagggctcgtccaaccaggcggatctttgcggctgtcatgtgccgcatccggattcacgttctcctcctttccgatggcatgggtgaggcaagctcctggaaaaggtctcgagtgggtatccaccataagtacttctggcgggcgcacttactacagggatagtgtgaagggaaggtttaccatttccagagataactcaaagaataccctgtacctgcaaatgaactccctcagggctgaagatacggctgtgtactactgtgctaaatttcgacaatactcaggtgggtttgactattggggccaggggacactggtgactgtgtcatcaagtcccaattccgcctcacatagcggaagtgccccacaaacttcctccgcgccaggttctcaagacattcaattgacgcagccgaatagcgtctctacgagcttggggagtaccgttaagctttcatgtactctttcctccggtaacatagagaacaactacgtgcattggtaccaactctacgagggcaggtctccaacgactatgatttacgatgacgataaacgacctgatggggtccctgaccgcttttcaggaagcattgacagaagctctaattccgcgtttttgactatccacaacgtagcgattgaagatgaagcaatttacttctgtcattcctatgtatccagctttaacgtcttcggaggtggaactaagttgacggtactccgagggggaggtgggtctcaccaccatcaccaccaccatcat

SEQ ID NO: 60 - DNA, 2004 bp, synthetic construct
  source 1..2004: mol_type = other DNA
atgtacaggatgcaactcctgtcttgcattgcactaagtcttgcacttgtcacgaattcgcagatccagctggtgcagtccggcccagagctgaaaaagccaggcgagaccgtgaagatctcctgcaaggccagcggctacactttcaagaattatggcatgaactgggtgaagcaggcccccggcaaggggctgaagtggatggggtggattaacacctataccggccagcccatttacgccaacgatttcaaggggagattcgcctttagcctggaaaccagcgcgagcaccgcctatctgcagatcaacaatcttaagaatgaggatacagccacctacttttgcgcccgggattggggcccatactggggccagggcaccctggtgatcgtgtccgcttcccccaattctgcctcccacagcggcagcgccccacagactagctccgcccccggttcccagaatatcatgatgacacagagcccttccagcctggccgtgtctgccggcgaaaaggtgaccgtgaactgcaagtctagccagtctgtgctgtacagctccaatcagatgaactatctggcctggtaccagcagaagcctggccagagccctaagctgctgatttactgggccagcacacgcgagtctggggtccctgacagatttaccggtagcggaagcggcacagatttcacactgacaatctcttctgtgcagaccgaagacctcgcagtgtactactgtctgcagtatctgagctcttggactttcggcggggggaccaagctggaaattaagggcggatctagtcggtcatcctccagtggtggtgggggtagcgggggaggcggtgctggacagccgctgcaagagcgggcacaagcctggggggaaagactcagggccagaatggaagaaatgggatcaagaacaagggacaggctcgatgaggtgaaggagcaagtagcagaggttcgagcaaagctggaagaacaagctcagcagattagattgcaagcagaggcggctcaagcacgactcaagagcaggttcgagccattggctgaagatatgcagcgacaatgggctggtcaggttgagaaagtccaagcggcggaagggaccagtgcagcaccggtaccatccgacaatcatggtggaagcagtagatcatccagttctggtgggggaggatccgggggcgggggtgaggttcagctccttgaatcaggcggagggctcgtccaaccaggcggatctttgcggctgtcatgtgccgcatccggattcacgttctcctcctttccgatggcatgggtgaggcaagctcctggaaaaggtctcgagtgggtatccaccataagtacttctggcgggcgcacttactacagggatagtgtgaagggaaggtttaccatttccagagataactcaaagaataccctgtacctgcaaatgaactccctcagggctgaagatacggctgtgtactactgtgctaaatttcgacaatactcaggtgggtttgactattggggccaggggacactggtgactgtgtcatcaagtcccaattccgcctcacatagcggaagtgccccacaaacttcctccgcgccaggttctcaagacattcaattgacgcagccgaatagcgtctctacgagcttggggagtaccgttaagctttcatgtactctttcctccggtaacatagagaacaactacgtgcattggtaccaactctacgagggcaggtctccaacgactatgatttacgatgacgataaacgacctgatggggtccctgaccgcttttcaggaagcattgacagaagctctaattccgcgtttttgactatccacaacgtagcgattgaagatgaagcaatttacttctgtcattcctatgtatccagctttaacgtcttcggaggtggaactaagttgacggtactccgagggggaggtgggtctcaccaccatcaccaccaccatcat

(19) United States
(12) Patent Application Publication
(10) Pub. No.: US 2025/0353931 A1
(43) Pub. Date: Nov. 20, 2025

(54) MULTI-SPECIFIC REAGENT FOR TARGETED DELIVERY OF LIPID NANOPARTICLES

(71) Applicant: Chimeris UK Ltd., Sawston (GB)

(72) Inventors: Biao Ma, Sawston (GB); Robert Williams, Sawston (GB); Jasvinder Hayre, Sawston (GB); Shimobi Onuoha, Sawston (GB)

(21) Appl. No.: 19/281,294

(22) Filed: Jul. 25, 2025

Related U.S. Application Data
(63) Continuation of application No. PCT/US2024/035305, filed on Jun. 24, 2024.
(60) Provisional application No. 63/510,566, filed on Jun. 27, 2023.

(51) Int. Cl. C07K 16/44; A61K 9/1271; A61K 9/51; A61K 40/11; A61K 40/31; A61K 40/42; C07K 16/18; C07K 16/28; C07K 16/30; C12N 15/88

(52) U.S. Cl. CPC C07K 16/44; A61K 9/1271; A61K 9/5123; A61K 40/11; A61K 40/31; A61K 40/4211; C07K 16/18; C07K 16/2809; C07K 16/30; C12N 15/88; C07K 2317/31; C07K 2317/622

(57) ABSTRACT

The present disclosure relates to a molecular delivery system that facilitates the internalization of LNPs into a specific target of choice, such as a specific cell type, ex vivo and/or in vivo. The present disclosure also relates to methods, molecules, and compositions for enhancing the targeted delivery of compounds within a living system. In particular, embodiments provided herein relate to methods, molecules, and compositions for the targeted delivery of lipid nanoparticles containing therapeutic molecules into a cell or system of choice, such as a T cell. The present disclosure also relates to methods of administering the enhanced targeting system to a patient or system, compositions for use in such methods, and further methods of use of the targeting system as part of T cell-based immunotherapy.

### RELATED APPLICATIONS AND INCORPORATION BY REFERENCE

This application is a continuation application of PCT Application Ser. No. PCT/US2024/035305, filed Jun. 24, 2024, which claims the benefit of U.S. Provisional Ser. No. 63/510,566, filed Jun. 27, 2023, which are hereby incorporated by reference in their entireties.

### REFERENCE TO SEQUENCE LISTING

The present application is being filed along with a Sequence Listing in electronic format. The Sequence Listing is provided as a file entitled CHMRS.004C1.xml, which was created and last modified on Jul. 24, 2025 and is 103,948 bytes in size. The information in the electronic Sequence Listing is hereby incorporated by reference in its entirety.

### FIELD

Aspects of the present disclosure described herein relate to methods, molecules, and compositions for enhancing the targeted delivery of compounds in a living system. In particular, embodiments provided herein relate to methods, molecules, and compositions for the targeted delivery of lipid nanoparticles containing a therapeutic molecule into a cell or system of choice, such as a T cell.

### BACKGROUND

A variety of cellular therapies have become standardized in the treatment of cancer. Specifically, immunotherapy is based on adoptive transfer of lymphocytes (e.g., T cells) into a patient. Among the many different types of immunotherapeutic agents, one of the most promising of the immunotherapeutic agents being developed is T cells expressing chimeric antigen receptors (CAR T cells). The chimeric antigen receptor (CAR) is a genetically engineered receptor that is designed to target a specific antigen, for example, a tumor antigen. This targeting can result in cytotoxicity against a tumor, for example, such that CAR T cells expressing CARs can target and kill tumors via the specific tumor antigens. This can include the infusion of polyclonal or antigen specific T-cells, lymphokine activated killer cells, natural killer cells, dendritic cells, or macrophages. Advancements have been made in the development of chimeric antigen receptor (CAR) bearing T-cells for adoptive T-cell therapies for cancer therapy, which are a promising therapeutic route for cancer immunotherapy and viral therapy.

CAR T-cell therapy is an immunotherapy in which the patient's own T-cells are isolated in a laboratory, genetically manipulated to express a synthetic receptor to recognize a particular antigen or protein and reinfused into the patient. A CAR can include several domains. For example, the CAR can have (1) an antigen-binding region, typically derived from an antibody, (2) a transmembrane domain to anchor the CAR into the T-cells, and/or (3) one or more intracellular T-cell signaling domains. First-generation CARs commonly incorporated a single chain variable fragment (scFv) that is derived from a monoclonal antibody (mAb) and a signaling motif from a TCR ζ chain. The second- and third-generation CARs are an improvement over the first-generation CARs with co-stimulatory activating motifs, which can lead to the enhanced proliferation, cytotoxicity, and persistence of the CAR bearing cells in vivo. Clinical trials have shown some evidence of anti-tumor activity, with insufficient activation, persistence, and homing to cancer tissue. Some anti-tumor responses have been reported in patients with B cell lymphoma, for example, and some neuroblastoma patients have reported partial response, stable disease, and remission. Second- and third-generation CAR-modified T-cells have been shown to be able to provide enhanced activation signals, proliferation, production of cytokines, and effector function of CAR-modified T-cells in pre-clinical trials. Initial clinical trials have been shown to exhibit some promising results.

Current adoptive T cell therapy for cancers can involve 1) the harvest of a patient's own T cells or those of a donor, 2) ex vivo genetic modification to express CARs in the expanded T cells, and/or 3) reintroduction of the engineered T cells into patient to fight off specific diseased cells. The complexity in manufacturing includes individualized T cell products for each patient, stringent quality control to release the products for human use, and most critically, associated high cost per individual, which prohibits wider applicability of adoptive T cell therapy. Recently the development of allogeneic adoptive T cell therapy has been gaining momentum, but significant challenges still remain, including in mass production of T cell products with high efficacy for general clinical use. The field would greatly benefit from off-the-shelf biological drugs that can rapidly educate the immune system to eliminate cancer and be produced in bulk quantities similar as conventional pharmaceuticals.

### SUMMARY

Described herein are compositions and methods for treating diseases, the compositions including protein constructs that facilitate the internalization of lipid nanoparticles (LNPs) into a specific target of choice, such as a specific cell type, ex vivo or in vivo.

Accordingly, some embodiments provided herein relate to molecular and/or protein constructs. Some embodiments provided herein relate to proteins with multi-specificity. In some embodiments, the protein has multi- and/or dual-specificity. In some embodiments, the protein with multi-specificity includes: (i) a first domain capable of binding a therapeutic molecule; and (ii) a second domain capable of binding a protein, cell, or tissue. In some embodiments, the therapeutic molecule is a lipid nanoparticle (LNP). In some embodiments, the LNP further includes a payload. In some embodiments, the payload is mRNA or DNA. In some embodiments, the mRNA or the DNA encodes for the expression of a chimeric antigen receptor (CAR) construct. In some embodiments, the first domain includes a derivative of an apolipoprotein, such as ApoE3. In some embodiments, the first domain includes a mutated and/or truncated ApoE3 domain. In some embodiments, the first domain includes an antibody variable (Fv) region-like polypeptide. In some embodiments, the antibody variable (Fv) region-like polypeptide has a high affinity for polyethylene glycol (PEG). In some embodiments, the antibody variable (Fv) region-like polypeptide has a high affinity for phosphatidylserine. In some embodiments, the antibody variable (Fv) region-like polypeptide has a high affinity for cholesterol or a derivative of cholesterol. In some embodiments, the first domain includes a peptide that binds to cholesterol or a derivative of cholesterol. In some embodiments, the second domain includes a polypeptide with binding affinity for a cellular protein antigen. In some embodiments, the second domain has an affinity for a cell surface antigen. In some embodiments, the second domain includes an antibody variable (Fv) region-like polypeptide. In some embodiments, the antibody variable (Fv) region-like polypeptide has a high affinity for at least one cell surface antigen. In some embodiments, the antibody variable (Fv) region-like polypeptide is a T cell receptor a subunit, a T cell receptor β subunit, a CD3, a CD4, a CD8, a CD5, and/or a CD28. In some embodiments, the protein further includes at least one linker. In some embodiments, the at least one linker includes a peptide linker. In some embodiments, the peptide linker includes a sequence with at least 80% identity to SEQ ID NO: 2. In some embodiments, the first domain includes a sequence with at least 80% identity to any one of the sequences of Table 5. In some embodiments, the second domain includes a sequence with at least 80% identity to any one of the sequences of Table 5. In some embodiments, the protein includes a sequence with at least 80% identity to any one of the sequences of Tables 3 and 5. In some embodiments, the protein has at least two binding targets. In some embodiments, the protein has three binding targets.

Some embodiments provided herein relate to a nucleotide encoding any one of the embodiments described herein.

Some embodiments provided herein relate to a nucleotide including a sequence with at least 80% identity to any one of the sequences of Tables 4 and 6.

Some embodiments disclosed herein relate to a vector encoding any one of the nucleotides of the embodiments disclosed herein, and/or capable of expressing any one of the proteins disclosed herein.

Some embodiments disclosed herein relate to a cell including any one of the nucleotides disclosed herein, the vector of any one of the embodiments of the present disclosure, and/or are capable of expressing any one of the proteins of the embodiments of the present disclosure.

Some embodiments disclosed herein relate to a composition including a multi- and/or dual-specific protein. In some embodiments, the protein includes a first domain capable of binding a therapeutic molecule; and a second domain capable of binding a protein, cell, or tissue. In some embodiments, the composition further includes the therapeutic molecule. In some embodiments, the therapeutic molecule is an mRNA or a DNA, and a pharmaceutically effective carrier. In some embodiments, the therapeutic molecule is a lipid nanoparticle (LNP). In some embodiments, the LNP further includes a payload. In some embodiments, the payload is an mRNA or a DNA. In some embodiments, the mRNA or the DNA encodes for the expression of a chimeric antigen receptor (CAR) construct. In some embodiments, the first domain includes a mutated and/or truncated ApoE3 domain. In some embodiments, the first domain includes an antibody variable (Fv) region-like polypeptide. In some embodiments, the antibody variable (Fv) region-like polypeptide has a high affinity for polyethylene glycol (PEG). In some embodiments, the antibody variable (Fv) region-like polypeptide has a high affinity for phosphatidylserine. In some embodiments, the antibody variable (Fv) region-like polypeptide has a high affinity for cholesterol or a derivative of cholesterol. In some embodiments, the first domain includes a peptide that binds to cholesterol or a derivative of cholesterol. In some embodiments, the second domain includes a polypeptide with binding affinity for a cellular protein antigen. In some embodiments, the second domain has an affinity for a cell surface antigen. In some embodiments, the second domain includes an antibody variable (Fv) region-like polypeptide. In some embodiments, the antibody variable (Fv) region-like polypeptide has a high affinity for at least one cell surface antigen. In some embodiments, the antibody variable (Fv) region-like polypeptide possesses binding affinity towards the T cell receptor a subunit, T cell receptor β subunit, CD3, CD4, CD8, CD5, and/or CD28. In some embodiments, the protein further includes at least one linker. In some embodiments, the at least one linker includes a peptide linker. In some embodiments, the peptide linker includes a sequence with at least 80% identity to SEQ ID NO: 2. In some embodiments, the first domain includes a sequence with at least 80% identity to any one of the sequences of Table 5. In some embodiments, the second domain includes a sequence with at least 80% identity to any one of the sequences of Table 5. In some embodiments, the protein includes a sequence with at least 80% identity to any one of the sequences of Tables 3 and 5. In some embodiments, the protein has at least two binding targets. In some embodiments, the protein has three binding targets.

Some embodiments disclosed herein relate to methods for treating a disease or disorder in a subject in need thereof. In some embodiments, the methods include administering to the subject any protein described herein, any nucleotide described herein, any vector described herein, any cell described herein, and/or any composition described herein. In some embodiments, the disease or disorder is a cancer. In some embodiments, the cancer is a blood cancer, lymphoma, multiple myeloma, leukemia, peripheral T cell lymphoma (PTCL), diffuse large B-cell lymphoma (DLBCL), follicular lymphoma, mantle cell lymphoma, multiple myeloma, B-cell acute lymphoblastic leukemia (ALL), Large B-cell lymphoma transformed from follicular lymphoma, High grade B-cell lymphoma, Aggressive B-cell lymphoma not otherwise specified (NOS), brain cancer (including but not limited to glioblastoma), lung cancer, ovarian cancer, breast cancer, prostate cancer, liver cancer, kidney cancer, stomach cancer, pancreatic cancer, or colon cancer. In some embodiments, the administration to the subject is conducted via intravenous or intra-tumoral injection. In some embodiments, the subject is mammalian and/or human.

Some embodiments disclosed herein relate to methods for treating a disease or disorder in a subject in need thereof. In some embodiments, the methods include administering a multi-specific protein comprising a first domain capable of binding a therapeutic molecule; and a second domain capable of binding a protein, cell, or tissue. In some embodiments, the disease or disorder is a cancer. In some embodiments, the cancer is a blood cancer, lymphoma, multiple myeloma, leukemia, peripheral T cell lymphoma (PTCL), diffuse large B-cell lymphoma (DLBCL), follicular lymphoma, mantle cell lymphoma, multiple myeloma, B-cell acute lymphoblastic leukemia (ALL), Large B-cell lymphoma transformed from follicular lymphoma, High grade B-cell lymphoma, Aggressive B-cell lymphoma not otherwise specified (NOS), Brain cancer (including but not limited to glioblastoma), lung cancer, ovarian cancer, breast cancer, prostate cancer, liver cancer, kidney cancer, stomach cancer, pancreatic cancer, or colon cancer. In some embodiments, the administration to the subject is conducted via intravenous or intra-tumoral injection. In some embodiments, the subject is mammalian and/or human. In some embodiments, the method further includes administering an effective dose of the therapeutic molecule. In some embodiments, the therapeutic molecule is an mRNA or a DNA, and a pharmaceutically effective carrier. In some embodiments, the therapeutic molecule is a lipid nanoparticle (LNP). In some embodiments, the LNP further includes a payload. In some embodiments, the payload is an mRNA or a DNA. In some embodiments, the mRNA or the DNA encodes for the expression of a chimeric antigen receptor (CAR) construct. In some embodiments, the first domain includes a mutated and/or truncated ApoE3 domain. In some embodiments, the first domain includes an antibody variable (Fv) region-like polypeptide. In some embodiments, the antibody variable (Fv) region-like polypeptide has a high affinity for polyethylene glycol (PEG). In some embodiments, the antibody variable (Fv) region-like polypeptide has a high affinity for phosphatidylserine. In some embodiments, the antibody variable (Fv) region-like polypeptide has a high affinity for cholesterol or a derivative of cholesterol. In some embodiments, the first domain includes a peptide that binds to cholesterol or a derivative of cholesterol. In some embodiments, the second domain includes a polypeptide with binding affinity for a cellular protein antigen.

In some embodiments, the second domain has an affinity for a cell surface antigen. In some embodiments, the second domain includes an antibody variable (Fv) region-like polypeptide. In some embodiments, the antibody variable (Fv) region-like polypeptide has a high affinity for at least one cell surface antigen. In some embodiments, the antibody variable (Fv) region-like polypeptide is a T cell receptor a subunit, a T cell receptor β subunit, a CD3, a CD4, a CD8, a CD5, and/or a CD28. In some embodiments, the protein further includes at least one linker. In some embodiments, the at least one linker includes a peptide linker. In some embodiments, the peptide linker includes a sequence with at least 80% identity to SEQ ID NO: 2. In some embodiments, the first domain includes a sequence with at least 80% identity to any one of the sequences of Table 5. In some embodiments, the second domain includes a sequence with at least 80% identity to any one of the sequences of Table 5. In some embodiments, the protein includes a sequence with at least 80% identity to any one of the sequences of Tables 3 and 5. In some embodiments, the protein has at least two binding targets. In some embodiments, the protein has three binding targets.

Some embodiments disclosed herein relate to a use of any protein as described herein, any nucleotide described herein, any vector described herein, any cell described herein, and/or any composition described herein, for treating a disease or disorder in a subject. In some embodiments, the disease or disorder is a cancer. In some embodiments, the cancer is a blood cancer, lymphoma, multiple myeloma, leukemia, peripheral T cell lymphoma (PTCL), diffuse large B-cell lymphoma (DLBCL), follicular lymphoma, mantle cell lymphoma, multiple myeloma, B-cell acute lymphoblastic leukemia (ALL), Large B-cell lymphoma transformed from follicular lymphoma, High grade B-cell lymphoma, Aggressive B-cell lymphoma not otherwise specified (NOS), Brain cancer (including but not limited to glioblastoma), lung cancer, ovarian cancer, breast cancer, prostate cancer, liver cancer, kidney cancer, stomach cancer, pancreatic cancer, or colon cancer. In some embodiments, the subject is mammalian and or human.

Some embodiments disclosed herein relate to a use for a multi-specific protein in treating a disease or disorder in a subject. In some embodiments, the multi-specific protein includes a first domain capable of binding a therapeutic molecule; and a second domain capable of binding a protein, cell, or tissue. In some embodiments, the disease or disorder is a cancer. In some embodiments, the cancer is a blood cancer, lymphoma, multiple myeloma, leukemia, peripheral T cell lymphoma (PTCL), diffuse large B-cell lymphoma (DLBCL), follicular lymphoma, mantle cell lymphoma, multiple myeloma, B-cell acute lymphoblastic leukemia (ALL), Large B-cell lymphoma transformed from follicular lymphoma, High grade B-cell lymphoma, Aggressive B-cell lymphoma not otherwise specified (NOS), Brain cancer (including but not limited to glioblastoma), lung cancer, ovarian cancer, breast cancer, prostate cancer, liver cancer, kidney cancer, stomach cancer, pancreatic cancer, or colon cancer. In some embodiments, the subject is mammalian and or human. In some embodiments, the use further includes an effective dose of the therapeutic molecule. In some embodiments, the therapeutic molecule is an mRNA or a DNA, and a pharmaceutically effective carrier. In some embodiments, the therapeutic molecule is a lipid nanoparticle (LNP). In some embodiments, the LNP further includes a payload. In some embodiments, the payload is an mRNA or a DNA. In some embodiments, the mRNA or the DNA encodes for the expression of a chimeric antigen receptor (CAR) construct). In some embodiments, the first domain includes a mutated and/or truncated ApoE3 domain. In some embodiments, the first domain includes an antibody variable (Fv) region-like polypeptide. In some embodiments, the antibody variable (Fv) region-like polypeptide has a high affinity for polyethylene glycol (PEG). In some embodiments, the antibody variable (Fv) region-like polypeptide has a high affinity for phosphatidylserine. In some embodiments, the antibody variable (Fv) region-like polypeptide has a high affinity for cholesterol or a derivative of cholesterol. In some embodiments, the first domain includes a peptide that binds to cholesterol or a derivative of cholesterol. In some embodiments, the second domain includes a polypeptide with binding affinity for a cellular protein antigen. In some embodiments, the second domain has an affinity for a cell surface antigen. In some embodiments, the second domain includes an antibody variable (Fv) region-like polypeptide. In some embodiments, the antibody variable (Fv) region-like polypeptide has a high affinity for at least one cell surface antigen. In some embodiments, the antibody variable (Fv) region-like polypeptide is a T cell receptor a subunit, a T cell receptor 3 subunit, a CD3, a CD4, a CD8, a CD5, and/or a CD28. In some embodiments, the protein further includes at least one linker. In some embodiments, the at least one linker includes a peptide linker. In some embodiments, the peptide linker includes a sequence with at least 80% identity to SEQ ID NO: 2. In some embodiments, the first domain includes a sequence with at least 80% identity to any one of the sequences of Table 5. In some embodiments, the second domain includes a sequence with at least 80% identity to any one of the sequences of Table 5. In some embodiments, the protein includes a sequence with at least 80% identity to any one of the sequences of Tables 3 and 5. In some embodiments, the protein has at least two binding targets. In some embodiments, the protein has three binding targets.

### BRIEF DESCRIPTION OF THE DRAWINGS

The foregoing and other features of the present disclosure will become more fully apparent from the following description, taken in conjunction with the accompanying drawings. Understanding that these drawings depict only some embodiments in accordance with the disclosure and are therefore, not to be considered limiting of its scope, the disclosure will be described with additional specificity and detail through use of the accompanying drawings.

FIG. 1 depicts a non-limiting example schematic of an embodiment of a lipid nanoparticle (LNP) composition, including an ionizable cationic lipid, a cholesterol, a helper lipid, and a PEG lipid.

FIG. 2 depicts a non-limiting example cartoon schematic of a multi-specific reagent. In this example, the cartoon schematic depicts a dual-specific reagent. This dual-specific reagent includes an LNP-binding component and a polypeptide with binding affinity for a cellular protein antigen. In some embodiments, the LNP-binding component is an anti-PEG single chain Fv.

FIGS. 3A-3D depict non-limiting example cartoon schematic of multi-specific reagents, utilizing binding components derived from native ApoE3 (FIG. 3A). Examples of such reagents are an ApoE3 protein (with mutations to reduce binding to LDL receptors) with a cell binding arm (FIG. 3B), or a mutated/truncated ApoE3 LNP-binding arm with a cell binding arm (FIG. 3C), or anti-PEG single chain Fv followed by mutated/truncated ApoE3 LNP-binding arm with a cell binding arm (FIG. 3D).

FIG. 4 depicts a non-limiting example cartoon schematic for the mechanism of action of a multi-specific reagent, in this case a dual specific reagent, in which the dual specific reagent interacts with both a cell and a LNP, thus facilitating the association of LNPs with a target cell antigen (for example, CD3 protein present on T Cells) and promoting the uptake, by internalization, of the LNP and its contents into the target cell.

FIG. 5 depicts a non-limiting example SDS-PAGE gel analysis of the multi-specific reagents CM04A, CM04P, and CM04B. Each sample was subjected to electrophoresis as either reduced (R) or non-reduced (NR) preparations.

FIG. 6 depicts a non-limiting example measurement of multi-specific reagents CM04A, CM04P, and CM04B binding to Jurkat T cells, as measured through the shift in APC fluorescence. As described in Example 2 below, Jurkat cells were incubated with 20 μl of either CHO cell culture medium alone (“control”), or CHO cell culture supernatant containing an expressed multi-specific reagent. After 2 hours of incubation at room temperature, cells were washed and stained with an APC-conjugated anti-His tag secondary antibody reagent.

FIG. 7 depicts a non-limiting example quantification of CM04A, CM04P, and CM04B binding to PEG-Biotin, as detected using an anti-HIS tag-HRP antibody.

FIGS. 8A-8B depict non-limiting example flow cytometry dot plots gated on live PBMCs, showing EGFP-positive T cells (Quadrant 2) for PBMCs treated with either LNPs alone (FIG. 8A), or LNPs plus purified CM04B protein (FIG. 8B).

FIGS. 9A-9D depict non-limiting example histogram plots for EGFP fluorescence within CD4/CD8+ T cell populations from starting PBMCs treated with LNPs alone (FIG. 9A), or LNPs plus CM04P (FIG. 9B), CM04A (FIG. 9C), or CM04B protein (FIG. 9D).

FIGS. 10A-10C depict non-limiting example histogram plots of EGFP fluorescence in live HepG2 liver cell populations treated with LNP+PBS (FIG. 10A), LNP+CM04A (FIG. 10B), or LNP+CM04B (FIG. 10C), as described in Example 5.

FIGS. 11A-11D depict non-limiting example cartoon schematics for multi-specific reagents with enhanced binding affinities. Example constructs include an anti-PEG scFv-ApoE3-lipid binding arm-Cell Binding scFv (FIG. 11A), an ApoE3-lipid binding arm-anti-PEG scFv-Cell Binding scFv (FIG. 11B), an ApoE3-lipid binding arm-anti-PEG scFv-Cell Binding VHH (FIG. 11C), and an anti-PEG scFv-ApoE3-lipid binding arm-Cell Binding VHH (FIG. 11D).

FIG. 12 depicts a non-limiting example SDS-PAGE gel analysis of the multi-specific reagents CM04A, CM04B, and CM04C. Each sample was subjected to electrophoresis as either reduced (R) or non-reduced (NR) preparations.

FIG. 13 depicts a non-limiting example quantification of CM04A, CM04B, and CM04C binding to lipid nanoparticles (LNPs) via enzyme-linked immunosorbent assay (ELISA). Briefly, an immunoassay plate was coated with anti-cholesterol polyclonal antibodies. LNPs (+LNP) or bovine serum albumin as negative control (−LNP ctl) were captured in different wells on the coated plate. Subsequently, CM04A, CM04B and CM04C in different concentrations were added to designated wells and the binding to captured LNPs or negative control was detected using an anti-HIS tag-HRP antibody. Data shown in the graph are specific-binding to LNPs of each protein as calculated by subtracting signals of (−LNP ctl) from those of (+LNP).

FIGS. 14A-14B depict non-limiting example quantifications of bioluminescence imaging signal in mice that receive intravenous injections of LNPs containing luciferase mRNA (LNP-Luc) alone or LNP-Luc plus dual targeting reagents that specifically bind to LNP and mouse CD3 (DT/LNP-Luc). Data shown are mean±SEM of the total flux reading of dissected spleen (FIG. 14A) and liver (FIG. 14B) from treated mice (n=3 for each treatment condition).

FIGS. 15A-15B depict non-limiting example of CD19-CAR expression as measured by staining with APC-labelled anti-FMC63 antibody, specific for the CAR, within the CD4/CD8+ T cell populations from untreated human PBMCs, PBMCs treated with LNP containing CD19-CAR-mRNA alone and the same LNPs plus CM04A, CM04B or CM04C at 0.2 μM. FIG. 15A shows histogram plots derived from one donor. FIG. 15B shows the percentage of CD19-CAR+ T cells in three donors, with the bar showing the mean of three donors.

FIGS. 16A-16C depict non-limiting examples of luciferase activity derived from Fluc-mRNA encapsulated in LNPs in freshly isolated T cells from donor 1 (FIG. 16A), donor 2 (FIG. 16B), and donor 3 (FIG. 16C). Briefly, LNPs carrying 40 ng Fluc-mRNA (LNPs-Fluc) were incubated with CM04A, CM04B or CM04C in different concentrations for 1 hr at room temperature. Subsequently, (LNPs-Fluc) alone or the mix of (LNPs-Fluc) and various proteins were added to 80,000 donor T cells for each treatment. The treated cells were cultured at 37° C., 5% CO2 for 24 hrs. The luciferase signals were assayed using ONE-Glo™ EX Luciferase Assay System and detected on Varioskan LUX Multimode Microplate Reader. Data shown in the graph are fold change of luciferase signals from treated T cells to background signal from untreated cells.

### DETAILED DESCRIPTION

Although the disclosure is described in various exemplary alternatives and implementations as provided herein, it should be understood that the various features, aspects, and functionality described in one or more of the individual alternatives are not limited in their applicability to the particular alternative with which they are described. Instead, they can be applied alone or in various combinations to one or more of the other alternatives of the embodiments described herein, whether the alternatives are described or whether the features are presented as being a part of the described alternative. The breadth and scope of the present disclosure should not be limited by any exemplary alternatives described or shown herein.

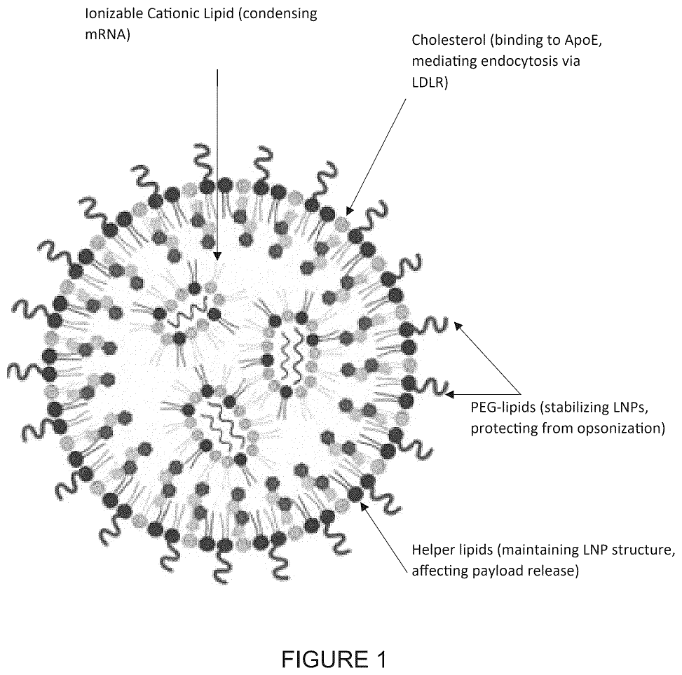

Disclosed herein are embodiments of a molecule with multi-binding specificity, such as an engineered protein, which facilitates the internalization of LNPs into a specific target of choice, such as a specific cell type, ex vivo or in vivo. A non-limiting schematic of a typical LNP is depicted in FIG. 1.

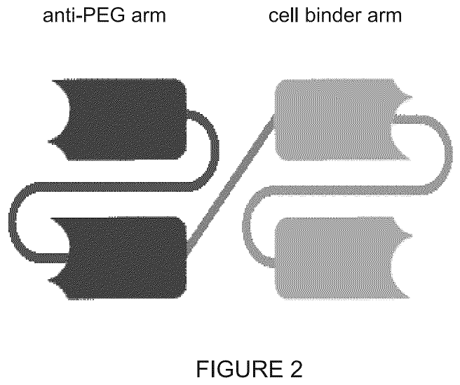

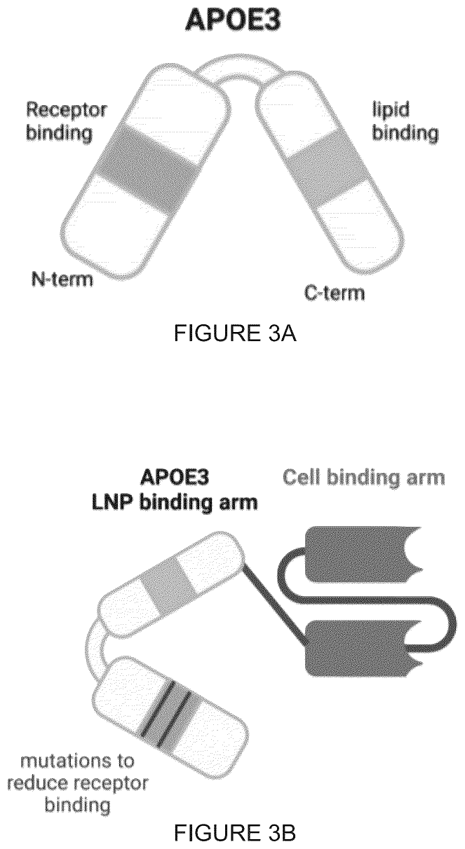

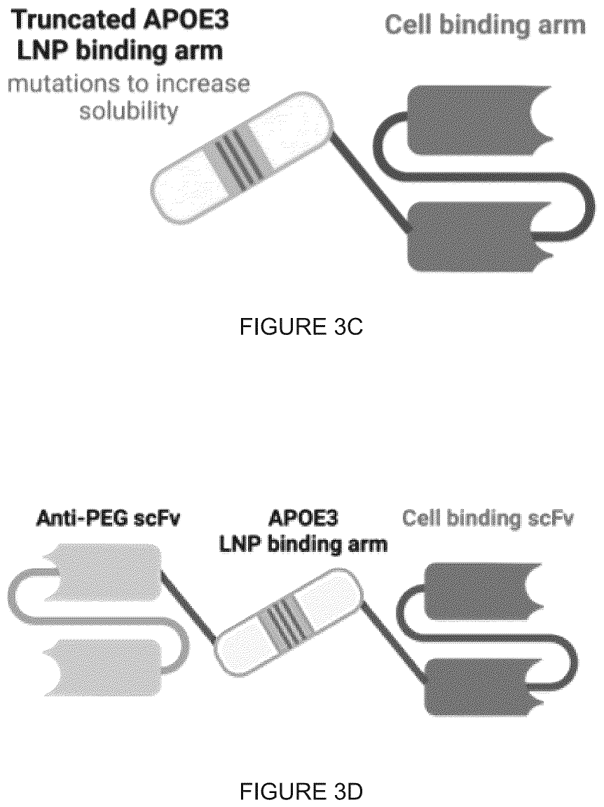

The present disclosure relates to compositions of such a molecule with multi-binding specificity, which in some embodiments is referred to as a “multi-specific reagent.” It will be understood that a “multi-specific reagent” refers to a molecule with at least two binding targets. In some embodiments, the molecule is a “dual-specific reagent,” which includes at least two binding domains: (1) a domain with specificity and high binding affinity to an LNP particle, and (2) a domain with specificity to a unique target protein or molecule present on a target cell and mediating efficient internalization upon binding. Example schematics of the dual-specific reagent are as shown in FIGS. 2 and 3A-3D. In some embodiments, the dual-specific reagent includes an LNP-binding component and a cell binding component. In some embodiments, the multi-specific reagent has more than two domains. In some embodiments, the multi-specific reagent has more than two binding targets. In some embodiments, the LNP-binding component includes a mutated or truncated ApoE3 domain. In some embodiments, the cell binding component includes a polypeptide with binding affinity for a cellular protein antigen.

In some embodiments, the molecule further includes additional domains/regions. In some embodiments, the additional domain/regions include a linker sequence connecting the binding domains.

In some embodiments, the first (LNP binding) domain is an antibody variable (Fv) region-like polypeptide with high affinity for polyethylene glycol (PEG), which could bind to PEGylated lipids present on the surface of an LNP. In some embodiments, the LNP binding domain is a full-length, truncated and/or mutated version of the “Apoprotein E3” protein, which is understood to bind to LNPs via its Lipid Binding Region. In some embodiments, the LNP binding region is an antibody Fv region-like polypeptide with high affinity for phosphatidylserine which is a key component of corresponding LNPs. In some embodiments, the LNP binding region is a peptide that binds to cholesterol or a derivative of cholesterol, such as hydroxycholesterol. Cholesterol is an essential component of LNPs.

In some embodiments, the second (target cell binding) domain includes any one of an Fv region-like polypeptide with affinity for unique T cell surface antigens. Non-limiting examples of an Fv region-like polypeptide with affinity for unique T cell surface antigens include T cell receptor α or β subunit, CD3, CD4, CD8, CD5, and CD28. In some embodiments, the target cell binding domains can mediate efficient internalization into target cells upon their engagement with cognate antigens.

In some embodiments, the binding domains of a multi-specific reagent are joined via a peptide linker such as listed in Table 2. In some embodiments, the peptide linker is Linker 2 as shown (SEQ ID NO: 2). In some embodiments, the multi-specific reagent includes at least one linker. In some embodiments, the multi-specific reagent includes at least two linkers.

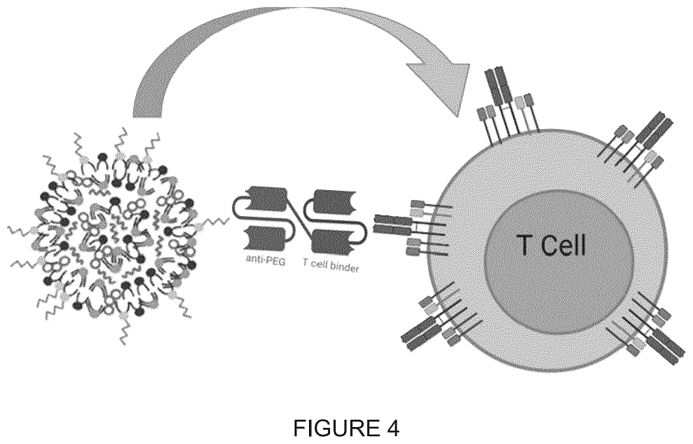

In some embodiments, one or more multi-specific reagent(s) is paired with any cognate LNPs for delivery into target cells of interest (FIG. 4). In some embodiments, the LNPs contain a payload. In some embodiments, the payload is in the form of nucleotides. In some embodiments, the payload is in the form of DNA. In some embodiments, the payload is in the form of mRNAs. In some embodiments, the DNA or mRNAs encode CARs. In some embodiments, the delivery of LNP-CAR-mRNA results in target cells expressing CARs, which in turn results in those cells exerting biological functions conferred by those CAR constructs, such as the killing of diseased cells.

Some embodiments disclosed herein relate to the ex vivo engineering of a certain type of patient-derived immune cells, such as T cells. In some embodiments, LNPs and multi-specific reagents can be added together into the culture medium of the cells, and the uptake of LNPs can be achieved through targeted internalizations.

Some embodiments disclosed herein relate to the in vivo engineering of a certain type of immune cells, such as T cells in a patient's body. In some embodiments, LNPs and multi-specific reagents can be combined in the buffer for infusion and infused into the blood stream of a patient. In some embodiments, the in vivo generation of CAR-T cells is achieved by infusion with LNP-CAR-mRNA together with multi specific reagents.

In some embodiments, the multi-specific reagents mediate targeted internalization of the LNP payload. In some embodiments, a cell or cells of interest express the payload of LNPs following administration of the multi-specific reagent. In some embodiments, this administration is used for producing in situ CAR-T cells.

In some embodiments, the one or more LNP binding domain of the multi-specific reagent includes a truncated and/or mutated ApoE3 domain. In some embodiments, the truncated and/or mutated ApoE3 domain can bind to cholesterols on the surface of LNPs, but lacks the ability to bind to Low-density lipoprotein receptor (LDLR) expressed on many types of human cells.

Intact ApoE3 is abundant in human blood and ApoE-LDLR interactions are responsible for the uptake, retention as well as clearance of LNPs in liver tissues. By decorating LNPs with truncated and mutated ApoE3 present in the multi specific reagents, the LNPs may be shielded from binding to intact ApoE3, prevented from interacting with high LDLR-expressing cells such as liver cells due to non-engagement, and in turn, retained in liver tissues to a much less degree than LNPs alone. In some embodiments, this mode of action reduces liver toxicity associated with LNP-based medicine and further enhances the delivery of LNPs to target cells of interest.

The multi-specific reagents described herein have many aspects of novelty in the field. Firstly, no multi targeting molecules have previously been designed with the function of binding to mRNA encapsulated in LNP (LNP-mRNA) and facilitating their delivery to a specific target cell of interest. There also have not been any reports for standalone multi-specific reagents that can be paired with any cognate LNP-mRNAs for in vivo administration. Such mode of action can make LNP-mRNA-based medicine more manufacturable than current means.

In the case of Apoprotein E (ApoE), prior to the present disclosure, the use of the protein (or domains thereof) in the specified forms as described herein has not been used for the purpose of binding to LNPs, or for bringing other binding domains into contact with LNPs. Using truncated and mutated ApoE3 to reduce the retention of LNPs in the liver tissue has not previously been reported.

In some embodiments, the protein has multi- and/or dual-specificity. In some embodiments, the protein with multi-specificity includes: (i) a first domain capable of binding a therapeutic molecule; and (ii) a second domain capable of binding a protein, cell, or tissue. In some embodiments, the second domain is capable of binding a protein. In some embodiments, the second domain is capable of binding a cell. In some embodiments, the second domain is capable of binding a tissue. In some embodiments, the second domain is capable of binding an epitope present in a subject.

In some embodiments, the therapeutic molecule is a lipid nanoparticle (LNP). In some embodiments, the LNP further includes a payload. In some embodiments, the payload is a therapeutic molecule. In some embodiments, the payload is a drug. In some embodiments, the payload is a protein sequence. In some embodiments, the payload is a nucleotide sequence. In some embodiments, the payload is an mRNA or a DNA. In some embodiments, the mRNA or the DNA encodes for the expression of a chimeric antigen receptor (CAR) construct).

In some embodiments, the first domain includes a derivative of an apolipoprotein. In some embodiments, the first domain includes a derivative of the apolipoprotein ApoE3. In some embodiments, the first domain includes a mutated and/or truncated ApoE3 domain. In some embodiments, the first domain includes an antibody variable (Fv) region-like polypeptide. In some embodiments, the antibody variable (Fv) region-like polypeptide has a high affinity for polyethylene glycol (PEG). In some embodiments, the antibody variable (Fv) region-like polypeptide has a high affinity for phosphatidylserine. In some embodiments, the antibody variable (Fv) region-like polypeptide has a high affinity for cholesterol or a derivative of cholesterol. In some embodiments, the first domain includes a peptide that binds to cholesterol or a derivative of cholesterol.

In some embodiments, the second domain includes a polypeptide with binding affinity for a cellular protein antigen. In some embodiments, the second domain has an affinity for a cell surface antigen. In some embodiments, the second domain includes an antibody variable (Fv) region-like polypeptide. In some embodiments, the antibody variable (Fv) region-like polypeptide has a high affinity for at least one cell surface antigen. In some embodiments, the antibody variable (Fv) region-like polypeptide is a T cell receptor α subunit, a T cell receptor β subunit, a CD3, a CD4, a CD8, a CD5 and/or a CD28.

In some embodiments, the protein further includes at least one linker. In some embodiments, the at least one linker includes a peptide linker. In some embodiments, the peptide linker includes a sequence with at least 80%, 85%, 90%, 95%, 99%, 100%, or any integer that is between 80% and 100%, identity to any one of the sequences of Table 2. In some embodiments, the peptide linker includes a sequence with at least 80%, 85%, 90%, 95%, 99%, 100%, or any integer that is between 80% and 100%, identity to SEQ ID NO: 2.

In some embodiments, the first domain includes a sequence with at least 80%, 85%, 90%, 95%, 99%, 100%, or any integer that is between 80% and 100%, identity to any one of the sequences of Table 5. In some embodiments, the second domain includes a sequence with at least 80%, 85%, 90%, 95%, 99%, 100%, or any integer that is between 80% and 100%, identity to any one of the sequences of Table 5. In some embodiments, the protein includes a sequence with at least 80%, 85%, 90%, 95%, 99%, 100%, or any integer that is between 80% and 100%, identity to any one of the sequences of Tables 3 and 5. In some embodiments, the protein has at least two binding targets. In some embodiments, the protein has three binding targets.

Some embodiments provided herein relate to a nucleotide encoding any one of the embodiments of the present disclosure. Some embodiments provided herein relate to a nucleotide comprising a sequence with at least 80%, 85%, 90%, 95%, 99%, 100%, or any integer that is between 80% and 100%, identity to any one of the sequences of Tables 4 and 6.

Some embodiments disclosed herein relate to a vector encoding any one of the nucleotides of the embodiments of the present disclosure, and/or capable of expressing any one of the proteins of the embodiments of the present disclosure.

Some embodiments disclosed herein relate to a cell comprising any one of the nucleotides of the embodiments of the present disclosure, the vector of any one of the embodiments of the present disclosure, and/or are capable of expressing any one of the proteins of the embodiments of the present disclosure.

Some embodiments disclosed herein relate to a composition comprising a multi- and/or dual-specific protein. In some embodiments, the protein includes a first domain capable of binding a therapeutic molecule; and a second domain capable of binding a protein, cell, or tissue. In some embodiments, the composition further includes the therapeutic molecule. In some embodiments, the therapeutic molecule is an mRNA or a DNA, and a pharmaceutically effective carrier. In some embodiments, the therapeutic molecule is a lipid nanoparticle (LNP). In some embodiments, the LNP further includes a payload. In some embodiments, the payload is an mRNA or a DNA. In some embodiments, the mRNA or the DNA encodes for the expression of a chimeric antigen receptor (CAR) construct. In some embodiments, the first domain includes a mutated and/or truncated ApoE3 domain. In some embodiments, the first domain includes an antibody variable (Fv) region-like polypeptide. In some embodiments, the antibody variable (Fv) region-like polypeptide has a high affinity for polyethylene glycol (PEG). In some embodiments, the antibody variable (Fv) region-like polypeptide has a high affinity for phosphatidylserine. In some embodiments, the antibody variable (Fv) region-like polypeptide has a high affinity for cholesterol or a derivative of cholesterol. In some embodiments, the first domain includes a peptide that binds to cholesterol or a derivative of cholesterol. In some embodiments, the second domain includes a polypeptide with binding affinity for a cellular protein antigen. In some embodiments, the second domain has an affinity for a cell surface antigen. In some embodiments, the second domain includes an antibody variable (Fv) region-like polypeptide. In some embodiments, the antibody variable (Fv) region-like polypeptide has a high affinity for at least one cell surface antigen. In some embodiments, the antibody variable (Fv) region-like polypeptide possesses binding affinity towards the T cell receptor α subunit, T cell receptor β subunit, CD3, CD4, CD8, CD5, and/or CD28. In some embodiments, the protein further includes an at least one linker. In some embodiments, the at least one linker includes a peptide linker. In some embodiments, the peptide linker includes a sequence with at least 80%, 85%, 90%, 95%, 99%, 100%, or any integer that is between 80% and 100%, identity to SEQ ID NO: 2. In some embodiments, the first domain includes a sequence with at least 80%, 85%, 90%, 95%, 99%, 100%, or any integer that is between 80% and 100%, identity to any one of the sequences of Table 5. In some embodiments, the second domain includes a sequence with at least 80%, 85%, 90%, 95%, 99%, 100%, or any integer that is between 80% and 100%, identity to any one of the sequences of Table 5. In some embodiments, the protein includes a sequence with at least 80%, 85%, 90%, 95%, 99%, 100%, or any integer that is between 80% and 100%, identity to any one of the sequences of Tables 3 and 5. In some embodiments, the protein has at least two binding targets. In some embodiments, the protein has three binding targets.

Some embodiments disclosed herein relate to a method for treating a disease or disorder in a subject in need thereof. In some embodiments, the method includes administering to the subject the protein of any one of the embodiments of the present disclosure, the nucleotide of any one of the embodiments of the present disclosure, the vector of any one of the embodiments of the present disclosure, the cell of any one of the embodiments of the present disclosure, and/or the composition of any one of the embodiments of the present disclosure. In some embodiments, the disease or disorder is a cancer. In some embodiments, the cancer is a blood cancer, lymphoma, multiple myeloma, leukemia, peripheral T cell lymphoma (PTCL), diffuse large B-cell lymphoma (DLBCL), follicular lymphoma, mantle cell lymphoma, multiple myeloma, B-cell acute lymphoblastic leukemia (ALL), Large B-cell lymphoma transformed from follicular lymphoma, High grade B-cell lymphoma, Aggressive B-cell lymphoma not otherwise specified (NOS), Brain cancer (including but not limited to glioblastoma), lung cancer, ovarian cancer, breast cancer, prostate cancer, liver cancer, kidney cancer, stomach cancer, pancreatic cancer, or colon cancer. In some embodiments, the administration to the subject is conducted via intravenous or intra-tumoral injection. In some embodiments, the subject is mammalian and/or human.

Some embodiments disclosed herein relate to a method for treating a disease or disorder in a subject in need thereof, the method comprising administering a multi-specific protein comprising a first domain capable of binding a therapeutic molecule; and a second domain capable of binding a protein, cell, or tissue. In some embodiments, the disease or disorder is a cancer. In some embodiments, the cancer is a blood cancer, lymphoma, multiple myeloma, leukemia, peripheral T cell lymphoma (PTCL)diffuse large B-cell lymphoma (DLBCL), follicular lymphoma, mantle cell lymphoma, multiple myeloma, B-cell acute lymphoblastic leukemia (ALL), Large B-cell lymphoma transformed from follicular lymphoma, High grade B-cell lymphoma, Aggressive B-cell lymphoma not otherwise specified (NOS), Brain cancer (including but not limited to glioblastoma), lung cancer, ovarian cancer, breast cancer, prostate cancer, liver cancer, kidney cancer, stomach cancer, pancreatic cancer, or colon cancer. In some embodiments, the administration to the subject is conducted via intravenous or intra-tumoral injection. In some embodiments, the subject is mammalian and/or human. In some embodiments, the method further includes administering an effective dose of the therapeutic molecule. In some embodiments, the therapeutic molecule is an mRNA or a DNA, and a pharmaceutically effective carrier. In some embodiments, the therapeutic molecule is a lipid nanoparticle (LNP). In some embodiments, the LNP further includes a payload. In some embodiments, the payload is an mRNA or a DNA. In some embodiments, the mRNA or the DNA encodes for the expression of a chimeric antigen receptor (CAR) construct). In some embodiments, the first domain includes a mutated and/or truncated ApoE3 domain. In some embodiments, the first domain includes an antibody variable (Fv) region-like polypeptide. In some embodiments, the antibody variable (Fv) region-like polypeptide has a high affinity for polyethylene glycol (PEG). In some embodiments, the antibody variable (Fv) region-like polypeptide has a high affinity for phosphatidylserine. In some embodiments, the antibody variable (Fv) region-like polypeptide has a high affinity for cholesterol or a derivative of cholesterol. In some embodiments, the first domain includes a peptide that binds to cholesterol or a derivative of cholesterol. In some embodiments, the second domain includes a polypeptide with binding affinity for a cellular protein antigen. In some embodiments, the second domain has an affinity for a cell surface antigen. In some embodiments, the second domain includes an antibody variable (Fv) region-like polypeptide. In some embodiments, the antibody variable (Fv) region-like polypeptide has a high affinity for at least one cell surface antigen. In some embodiments, the antibody variable (Fv) region-like polypeptide is a T cell receptor α subunit, a T cell receptor β subunit, a CD3, a CD4, a CD8, a CD5 and/or a CD28. In some embodiments, the protein further includes at least one linker. In some embodiments, the at least one linker includes a peptide linker. In some embodiments, the peptide linker includes a sequence with at least 80%, 85%, 90%, 95%, 99%, 100%, or any integer that is between 80% and 100%, identity to SEQ ID NO: 2. In some embodiments, the first domain includes a sequence with at least 80%, 85%, 90%, 95%, 99%, 100%, or any integer that is between 80% and 100%, identity to any one of the sequences of Table 5. In some embodiments, the second domain includes a sequence with at least 80%, 85%, 90%, 95%, 99%, 100%, or any integer that is between 80% and 100%, identity to any one of the sequences of Table 5. In some embodiments, the protein includes a sequence with at least 80%, 85%, 90%, 95%, 99%, 100%, or any integer that is between 80% and 100%, identity to any one of the sequences of Tables 3 and 5. In some embodiments, the protein has at least two binding targets. In some embodiments, the protein has three binding targets.

Some embodiments disclosed herein relate to a use of the protein of any one of the embodiments of the present disclosure, the nucleotide of any one of the embodiments of the present disclosure, the vector any one of the embodiments of the present disclosure, the cell of any one of the embodiments of the present disclosure, and/or the composition of any one of the embodiments of the present disclosure, for treating a disease or disorder in a subject. In some embodiments, the disease or disorder is a cancer. In some embodiments, the cancer is a blood cancer, lymphoma, multiple myeloma, leukemia, peripheral T cell lymphoma (PTCL), diffuse large B-cell lymphoma (DLBCL), follicular lymphoma, mantle cell lymphoma, multiple myeloma, B-cell acute lymphoblastic leukemia (ALL), Large B-cell lymphoma transformed from follicular lymphoma, High grade B-cell lymphoma, Aggressive B-cell lymphoma not otherwise specified (NOS), Brain cancer (including but not limited to glioblastoma), lung cancer, ovarian cancer, breast cancer, prostate cancer, liver cancer, kidney cancer, stomach cancer, pancreatic cancer, or colon cancer. In some embodiments, the subject is mammalian and or human.

Some embodiments disclosed herein relate to a use for a multi-specific protein in treating a disease or disorder in a subject. In some embodiments, the multi-specific protein includes a first domain capable of binding a therapeutic molecule; and a second domain capable of binding a protein, cell, or tissue. In some embodiments, the disease or disorder is a cancer. In some embodiments, the cancer is a blood cancer, lymphoma, multiple myeloma, leukemia, peripheral T cell lymphoma (PTCL), diffuse large B-cell lymphoma (DLBCL), follicular lymphoma, mantle cell lymphoma, multiple myeloma, B-cell acute lymphoblastic leukemia (ALL), Large B-cell lymphoma transformed from follicular lymphoma, High grade B-cell lymphoma, Aggressive B-cell lymphoma not otherwise specified (NOS), Brain cancer (including but not limited to glioblastoma), lung cancer, ovarian cancer, breast cancer, prostate cancer, liver cancer, kidney cancer, stomach cancer, pancreatic cancer, or colon cancer. In some embodiments, the subject is mammalian and or human. In some embodiments, the use further includes an effective dose of the therapeutic molecule. In some embodiments, the therapeutic molecule is an mRNA or a DNA, and a pharmaceutically effective carrier. In some embodiments, the therapeutic molecule is a lipid nanoparticle (LNP). In some embodiments, the LNP further includes a payload. In some embodiments, the payload is an mRNA or a DNA. In some embodiments, the mRNA or the DNA encodes for the expression of a chimeric antigen receptor (CAR) construct). In some embodiments, the first domain includes a mutated and/or truncated ApoE3 domain. In some embodiments, the first domain includes an antibody variable (Fv) region-like polypeptide. In some embodiments, the antibody variable (Fv) region-like polypeptide has a high affinity for polyethylene glycol (PEG). In some embodiments, the antibody variable (Fv) region-like polypeptide has a high affinity for phosphatidylserine. In some embodiments, the antibody variable (Fv) region-like polypeptide has a high affinity for cholesterol or a derivative of cholesterol. In some embodiments, the first domain includes a peptide that binds to cholesterol or a derivative of cholesterol. In some embodiments, the second domain includes a polypeptide with binding affinity for a cellular protein antigen. In some embodiments, the second domain has an affinity for a cell surface antigen. In some embodiments, the second domain includes an antibody variable (Fv) region-like polypeptide. In some embodiments, the antibody variable (Fv) region-like polypeptide has a high affinity for at least one cell surface antigen. In some embodiments, the antibody variable (Fv) region-like polypeptide is a T cell receptor α subunit, a T cell receptor 3 subunit, a CD3, a CD4, a CD8, a CD5 and/or a CD28. In some embodiments, the protein further includes at least one linker. In some embodiments, the at least one linker includes a peptide linker. In some embodiments, the peptide linker includes a sequence with at least 80%, 85%, 90%, 95%, 99%, 100%, or any integer that is between 80% and 100%, identity to SEQ ID NO: 2. In some embodiments, the first domain includes a sequence with at least 80%, 85%, 90%, 95%, 99%, 100%, or any integer that is between 80% and 100%, identity to any one of the sequences of Table 5. In some embodiments, the second domain includes a sequence with at least 80%, 85%, 90%, 95%, 99%, 100%, or any integer that is between 80% and 100%, identity to any one of the sequences of Table 5. In some embodiments, the protein includes a sequence with at least 80%, 85%, 90%, 95%, 99%, 100%, or any integer that is between 80% and 100%, identity to any one of the sequences of Tables 3 and 5. In some embodiments, the protein has at least two binding targets. In some embodiments, the protein has three binding targets.

### BACKGROUND

Lipid nanoparticles (LNPs) with an mRNA payload have been successfully applied in creating vaccines against COVID-19. Currently, there are many ongoing clinical programs worldwide in the evaluation of personalized cancer vaccines and gene therapy drugs based on LNPs-mRNA. It has been shown recently that LNPs which encapsulate mRNAs encoding CARs can be delivered into T cells in mouse models and the resulting in vivo-generated CAR-T cells can kill diseased cells and mediate recovery with some efficiency. This route of therapy is considered safe as the expression of mRNA in vivo is rapid and transient with little evidence of genome modification through gene integration. Repeated dosing may be possible to sustain long term clinical benefit. This technology has significantly advanced the creation of a genuine off-the-shelf CAR-product for in vivo applications. However, one major obstacle that this technology faces is to achieve targeted delivery of LNPs-CAR-mRNAs into T cells with significantly higher specificity and efficiency than to non-T-immune cells as well as other types of cells, including liver cells. The most used method to generate T cell-targeted LNPs-CAR-mRNAs is the chemical conjugation of specific antibodies or ligands against T cell markers such as CD4, CD8, CD3, and CD5 to a type of modified lipids located on the surface of LNPs. In the in vivo setting, these antibody-decorated LNPs can bind to circulating T cells specifically, which leads to the internalization into T cells and expression of mRNA payload. The chemical conjugation technology faces various challenges. For example, chemical conjugation is a multi-step process that involves the post-production modification of both LNPs and antibodies followed by a carefully controlled conjugation reaction. Difficulties exist to scale up this process for the purpose of commercial manufacturing. For LNPs that are conjugated to different T-cell specific antibodies, different production processes must be established for each product, which may limit their manufacturability. Lastly, it is well known that liver tissue plays an essential role in LNPs clearance due to the nature of their lipid compositions. When the LNPs with mRNA payload are administered through I.V. injection, significant accumulation of LNPs and expression from their mRNA cargos are frequently seen in liver tissues. Even though chemically conjugating T cell-specific antibodies to LNPs can enhance the targeted delivery to T cells, the issues linked to liver accumulation and potentially, liver toxicity are not addressed.

The systems and compositions disclosed herein for standalone multi-specific reagents for targeted delivery of LNPs with mRNA payload to immune cells address these issues associated with the in vivo drug delivery technology. Three features of these reagents have been formulated to achieve efficient targeted delivery to this cell type, including: high affinity binding to LNPs; high specificity toward T cell surface marker; and high efficiency in mediating internalization to T cells. Such features can be adapted to target other types of immune cells if suitable specific cell markers and antibodies are chosen. The standalone multi specific reagents can be produced independently from LNPs, and their manufacturing may be achieved using existing industrial processes that have been established in producing bi-specific antibody drugs. The standalone reagents allow pairing to any suitable LNPs, which may be beneficial for repeated dosing regimen by adopting different LNPs:multi-specific reagents combinations in the treatment process to potentially lower the frequency of treatment associated adverse effects. Further, if the LNPs-binding moiety of the multi-specific reagents can attenuate the retention of LNPs in the liver tissue by disrupting the LNPs' binding to lipoprotein receptors on liver cells, it can further enhance the targeted delivery to immune cells and reduce liver toxicity associated with LNP- or liposome-based drugs.

The molecular delivery systems and compositions of the present disclosure result in specific LNP delivery into specific cells, including, into T lymphocytes, in order to deliver genetic information into those cells, for example nucleic acid sequences that encode Chimeric Antigen Receptors (CARs) or associated modules. This specific LNP-mediated delivery results in the expression of CARs or other proteins by the cell. The advantages of the compositions, systems, and methods described herein can be broken down into two areas: (1) the potential to target genetic material more efficiently/cheaply/easily into primary T cells via LNPs ex vivo vs other existing methods such as virus-mediated delivery or electroporation; and (2) the potential to target LNP-incorporated genetic material into primary T cells in vivo with higher efficiency and/or specificity than existing methods. In particular, increasing specificity for T cell targeting vs non-T cells (such as liver cells or other immune cells) reduces the potential for toxicity caused by off-target introduction of genetic material.

There is currently little evidence in the field of other attempts to target LNP-incorporated genetic material, such as mRNA, specifically into target cells using engineered protein molecules which bind to an antigen on the target cell type.

### Definitions

The following definitions are provided to facilitate understanding of the alternatives or alternatives of the embodiments described herein. Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed.

Some embodiments herein relate to a molecule and/or protein with multi-specificity. “Multi-specific” as used herein has its ordinary meaning as understood in light of the specification, and refers to a molecule and/or protein capable of binding to more than one target. In some embodiments, one target is a therapeutic molecule. In some embodiments, one target is a cell, protein, or tissue.

Some embodiments herein relate to a molecule and/or protein with dual specificity. “Dual-specific” as used herein has its ordinary meaning as understood in light of the specification, and refers to a molecule and/or protein capable of binding to at least two targets. In some embodiments, one target is a therapeutic molecule. In some embodiments, one target is a cell, protein, or tissue.

As used herein, “a” or “an” may mean one or more.

As used herein, “about” in reference to a numeric value, including, for example, whole numbers, fractions, and percentages, generally refers to a range of numerical values (e.g., +/−5% to 10% of the recited value) that one of ordinary skill in the art would consider equivalent to the recited value (e.g., having the same function or result).

The term “comprising” as used herein is synonymous with “including,” “containing,” or “characterized by,” and is inclusive or open-ended and does not exclude additional, unrecited elements or method steps.

The term “in vitro” as used herein has its ordinary meaning as understood in light of the specification, and refers to a system or condition in a cell, tissue, or organ outside of a subject's body. In some embodiments, the cell, tissue, or organ is not a primary cell, tissue, or organ taken directly from the subject. In some embodiments, the cell is an established cell line. In some embodiments, the cell is derived from a primary cell.

The term “ex vivo” as used herein has its ordinary meaning as understood in light of the specification, and refers to a system or condition in a cell, tissue, or organ outside of a subject's body, which is later returned to the subject's body.

The term “in vivo” as used herein has its ordinary meaning as understood in light of the specification, and refers to a system or condition within a subject's body.

The term “in situ” as used herein has its ordinary meaning as understood in light of the specification, and refers to a place of origin. For example, “carcinoma in situ” refers to cancer cells found only in the place where they first formed.

The terms “primary cell,” “primary tissue,” and “primary organ” have their ordinary meaning as understood in light of the specification, and refer to a cell, tissue, or organ, respectively, that has been directly taken from a subject.

As used herein, “nucleic acid” or “nucleic acid molecule” have their ordinary meaning as understood in light of the specification, and refers to polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Nucleic acid molecules can include monomers that are naturally occurring nucleotides (such as DNA and RNA), or analogs of naturally occurring nucleotides (e.g., enantiomeric forms of naturally occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. The term “nucleic acid molecule” also includes so-called “peptide nucleic acids,” which include naturally occurring or modified nucleic acid bases attached to a polyamide backbone. Nucleic acids can be either single stranded or double stranded. In some embodiments, a nucleic acid encoding a chimeric antigen receptor is provided. In some embodiments, a method of making a nucleic acid encoding a chimeric antigen receptor is provided. In some embodiments, a nucleic acid encoding a chimeric antigen receptor specific for a ligand on a B cell is provided. In some embodiments, a nucleic acid encoding a chimeric antigen receptor specific for a ligand on a tumor cell is provided. In some embodiments, the nucleic acid is a DNA encoding a chimeric antigen receptor. In some embodiments, the nucleic acid is an mRNA encoding a chimeric antigen receptor. In some embodiments, the chimeric antigen receptor is bi-specific.

“Vector” as described herein, has its ordinary meaning as understood in light of the specification, and is a nucleic acid vehicle that carries a generic material encoding a protein or mRNA of interest into another cell, such that it is replicated and/or expressed in the cell. There are several types of vectors. Without being limiting, a vector can be a plasmid, viral vector, cosmid, artificial chromosome, or an mRNA. The vector can be linear or circular. In some embodiments provided herein, a viral vector is used to carry the nucleic acid encoding a chimeric antigen receptor. In some embodiments, the viral vector is a lentiviral vector. In some embodiments, the viral vector is a retroviral vector. In some embodiments, the viral vector is a gammaretroviral vector. In some embodiments, the vector is a foamy viral vector. In some embodiments, the vector is a plasmid. In some embodiments, the vector is an mRNA. In some embodiments, the vector is linear and includes telomeres.

An “expression cassette” as described herein, has its ordinary meaning as understood in light of the specification, and refers to a gene operatively linked to a regulatory sequence. Without being limiting, transduction or transfection of an expression cassette into a cell may result in the successful expression of the gene's encoded protein.

“Plasmid” as described herein, has its ordinary meaning as understood in light of the specification, and is a genetic structure in a cell that can replicate independently of the chromosomes. Without being limiting, the plasmid can be a small circular DNA strand in the cytoplasm of a bacterium or protozoan, or a linear nucleic acid.

“Express” or “expression” as described herein have their ordinary meaning as understood in light of the specification, and thus refer to the presence of a molecule in a living system. For example, “gene expression” refers to the transcription and translation of a DNA gene into first an RNA, and then a protein. Similarly, “protein expression” refers to the synthesis, and subsequent presence, of a protein within a system. It will be therefore understood that if a cell is said to “express” protein A, that cell is capable of producing protein A.

The terms “polypeptide”, “peptide”, “protein,” and “protein construct” are used interchangeably herein to refer to polymers of amino acids of any length. The polymer may be linear, cyclic, or branched, it may include modified amino acids, and it may be interrupted by non-amino acids. The terms also encompass amino acid polymers that have been modified, for example, via sulfation, glycosylation, lipidation, acetylation, phosphorylation, iodination, methylation, oxidation, proteolytic processing, phosphorylation, prenylation, racemization, selenoylation, transfer-RNA mediated addition of amino acids to proteins such as arginylation, ubiquitination, or any other manipulation, such as conjugation with a labeling component. In some embodiments, the polypeptides may contain modifications with respect to a native or natural sequence, as long as the protein maintains the desired activity. These modifications may be deliberate, as through site-directed mutagenesis, or may be accidental, such as through mutations of hosts which produce the proteins or errors due to PCR amplification. Standard amino acids can be written in their full name, three letter name, or one letter name; for example: Histidine, His, or H. Non-limiting examples of amino acids include: histidine, lysine, methionine, phenylalanine, threonine, tryptophane, asparagine, aspartic acid/aspartate, alanine, arginine, cysteine, glutamic acid/glutamate, glutamine, glycine, proline, serine, and tyrosine.

As used herein the term “amino acid” has its ordinary meaning as understood in light of the specification refers to either natural and/or unnatural or synthetic amino acids, including glycine and both the D or L optical isomers, and amino acid analogs and peptidomimetics. For purposes of classifying amino acids substitutions as conservative or nonconservative, amino acids are grouped as follows: Group I (hydrophobic side chains): met, ala, val, leu, ile; Group II (neutral hydrophilic side chains): cys, ser, thr; Group III (acidic side chains): asp, glu; Group IV (basic side chains): asn, gln, his, lys, arg; Group V (residues influencing chain orientation): gly, pro; and Group VI (aromatic side chains): trp, tyr, phe. Conservative substitutions involve substitutions between amino acids in the same class. Non-conservative substitutions constitute exchanging a member of one of these classes for a member of another.

A polypeptide or amino acid sequence “derived from” a designated protein refers to the origin of the polypeptide. Preferably, the polypeptide has an amino acid sequence that is essentially identical to that of a polypeptide encoded in the sequence, or a portion thereof wherein the portion consists of at least 10-20 amino acids, or at least 20-30 amino acids, or at least 30-50 amino acids, or which is immunologically identifiable with a polypeptide encoded in the sequence. This terminology also includes a polypeptide expressed from a designated nucleic acid sequence. Peptide sequences having at least 80%, 85%, 90%, 95%, 99%, or 100% homology to any one of the peptide sequences disclosed herein and having the same or similar functional properties are envisioned. The percent homology may be determined according to amino acid substitutions, deletions, or additions between two peptide sequences. Peptide sequences having some percent homology to any one of the peptide sequences disclosed herein may be produced and tested by one skilled in the art through conventional methods. The % homology or % identity of two sequences is well understood in the art and can be calculated by the number of conserved amino acids or nucleotides relative to the length of the sequences.

A protein “domain” is a select region of a protein. A domain may be conserved through related proteins. In some embodiments, the protein domain is self-stabilizing and forms independently from the rest of the protein. For example, in an IL-12 protein, p35 and p40 are both considered subdomains of IL-12. A “subdomain” is a smaller, distinct region within a domain. For example, a region within a p35 sequence would be a p35 subdomain.

“Polymer” refers to a series of monomer groups linked together. A polymer is composed of multiple units of a single monomer (a homopolymer) or different monomers (a heteropolymer). High MW polymers are prepared from monomers that include, but are not limited to, acrylates, methacrylates, acrylamides, methacrylamides, styrenes, vinyl-pyridine, vinyl-pyrrolidone and vinyl esters such as vinyl acetate. Additional monomers are useful in high MW polymers. When two different monomers are used, the two monomers are called “comonomers,” meaning that the different monomers are copolymerized to form a single polymer. The polymer can be linear or branched. When the polymer is branched, each polymer chain is referred to as a “polymer arm.” The end of the polymer arm linked to the initiator moiety is the proximal end, and the growing-chain end of the polymer arm is the distal end. On the growing chain-end of the polymer arm, the polymer arm end group can be the radical scavenger, or another group.

A “monomer” refers to a single protein. A “polymer” refers to more than one protein connected together through an at least one chemical bond. An “interface” refers to the amino acid region(s) that are connected together.

A “motif” refers to the primary structure of a strand of nucleotides or amino acids. Non-limiting examples of a nucleotide motif include a stem-loop, G-quadruplex, and D-loop. Non-limiting examples of a protein motif include a beta hairpin, a Greek key, omega loop, helix-loop-helix, zinc finger, helix-turn-helix, nest, and niche motif.

A “chemical linker” refers to a chemical moiety that links two groups together, such as a half-life extending moiety and a protein. The linker can be cleavable or non-cleavable. Cleavable linkers can be hydrolyzable, enzymatically cleavable, pH sensitive, photolabile, or disulfide linkers, among others. Other linkers include homobifunctional and heterobifunctional linkers. A “linking group” is a functional group capable of forming a covalent linkage consisting of one or more bonds to a bioactive agent.

A “peptide linker” refers to a chemical linker that includes a series of peptides. In some embodiments, the peptide linker is Linker 2 as shown (SEQ ID NO: 2).

A “lipid nanoparticle,” or “LNP,” refers to a small molecule including lipids. A non-limiting representative schematic of an LNP can be found in FIG. 1. In some embodiments, the LNP includes a payload. In some embodiments, the payload is therapeutic. In some embodiments, the payload is a drug and/or a small molecule. In some embodiments, the payload is a nucleotide sequence. In some embodiments, the payload is a DNA. In some embodiments, the payload is an mRNA. In some embodiments, the payload is an mRNA encoding a CAR construct.

The term “polyethylene glycol,” or “PEG,” refers to a molecular chain including polyethylene glycol. PEG can be monodispersed or polydispersed. PEG can be attached with a variety of functional groups such as Azide, Amine, NHS active ester, Alkyne, DBCO, Maleimide, Biotin, and DSPE. In some embodiments, PEG is used as part of a system for nanoparticle drug delivery. In some embodiments, PEG is part of a composition including a high transition temperature phospholipid, a PEG lipid, ionizable lipid, and a helper lipid.

“Phosphatidylserine” refers to an anionic phospholipid. In some embodiments, phosphatidylserine is modified. In some embodiments, the phosphatidylserine includes the structure of formula (I):

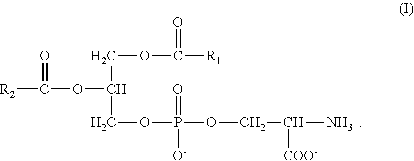

“Cholesterol” refers to a steroid compound. A “steroid” refers to a compound with a core structure of three six-member cyclohexane rings and one five-member cyclopentane ring. In some embodiments, steroids function as signaling molecules. In some embodiments, steroids alter cell membrane fluidity. In some embodiments, cholesterol is capable of intercalating into a cell membrane. In some embodiments, cholesterol is modified. In some embodiments, cholesterol includes the structure of formula (II):

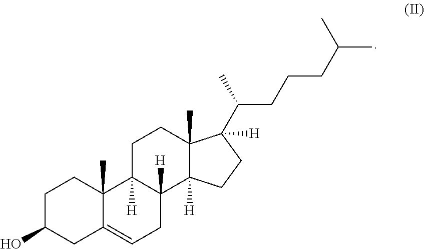

The term “reactive group” refers to a group that is capable of reacting with another chemical group to form a covalent bond, for example, is covalently reactive under suitable reaction conditions, and generally represents a point of attachment for another substance. The reactive group may be a moiety, such as maleimide or succinimidyl ester, capable of chemically reacting with a functional group on a different moiety to form a covalent linkage. Reactive groups generally include nucleophiles, electrophiles, and photoactivatable groups.

“Molecular weight” in the context of the polymer can be expressed as either a number average molecular weight, or a weight average molecular weight or a peak molecular weight. Unless otherwise indicated, all references to molecular weight herein refer to the peak molecular weight. These molecular weight determinations, number average (Mn), weight average (Mw) and peak (Mp), can be measured using size exclusion chromatography or other liquid chromatography techniques. Other methods for measuring molecular weight values can also be used, such as the use of end-group analysis or the measurement of colligative properties (e.g., freezing-point depression, boiling-point elevation, or osmotic pressure) to determine number average molecular weight, or the use of light scattering techniques, ultracentrifugation, or viscometry to determine weight average molecular weight. In some embodiments, the molecular weight is measured by SEC-MALS (size exclusion chromatography—multi angle light scattering). In some embodiments, the polymeric reagents are typically polydisperse (for example, number average molecular weight and weight average molecular weight of the polymers are not equal), and can possess low polydispersity values of, for example, less than about 1.5, as judged, for example, by the PDI value derived from the SEC-MALS measurement. In some embodiments, the polydispersities (PDI) are in the range of about 1.4 to about 1.2. In some embodiments the PDI is less than about 1.15, 1.10, 1.05, or 1.03.

Percentage sequence identities are determined with antibody sequences maximally aligned by the Kabat numbering convention for a variable region or EU numbering for a constant region. After alignment, if a subject antibody region (e.g., the entire mature variable region of a heavy or light chain) is being compared with the same region of a reference antibody, the percentage sequence identity between the subject and reference antibody regions is the number of positions occupied by the same amino acid in both the subject and reference antibody region divided by the total number of aligned positions of the two regions, with gaps not counted, multiplied by 100 to convert to percentage. Sequence identities of other sequences can be determined by aligning sequences using algorithms, such as BESTFIT, FASTA, and TFASTA in the Wisconsin Genetics Software Package Release 7.0, Genetics Computer Group, 575 Science Dr., Madison, WI, using default gap parameters, or by inspection, and the best alignment (for example, resulting in the highest percentage of sequence similarity over a comparison window). Percentage of sequence identity is calculated by comparing two optimally aligned sequences over a window of comparison, determining the number of positions at which the identical residues occur in both sequences to yield the number of matched positions, dividing the number of matched positions by the total number of positions in the window of comparison (for example, the window size), and multiplying the result by 100 to yield the percentage of sequence identity.

“Ligand” as described herein, has its ordinary meaning as understood in light of the specification, and refers to a substance that can form a complex with a biomolecule. By way of example and not of limitation, ligands can include substrates, proteins, small molecules, inhibitors, activators, nucleic acids, and neurotransmitters. Binding can occur through intermolecular forces, for example ionic bonds, hydrogen bonds, and van der walls interactions. Ligand binding to a receptor protein can alter the three-dimensional structure and determine its functional state. The strength of binding of a ligand is referred to as the binding affinity and can be determined by direct interactions and solvent effects. A ligand can be bound by a “ligand binding domain.” A ligand binding domain, for example, can refer to a conserved sequence in a structure that can bind a specific ligand or a specific epitope on a protein. The ligand binding domain or ligand binding portion can include an antibody or binding fragment thereof or scFv, a receptor ligand or mutants thereof, peptide, and/or polypeptide affinity molecule or binding partner. Without being limiting, a ligand binding domain can be a specific protein domain or an epitope on a protein that is specific for a ligand or ligands.

“Affinity” has its ordinary meaning as understood in light of the specification, and refers to the strength by which two molecules bind. Therefore, something with a low affinity has a weak bond, while something with a high affinity has a strong bond.

“Protein function” has its ordinary meaning as understood in light of the specification, and refers to the activity of a given protein. For example, if a protein is capable of activating a cellular signal, then that activation is its function. Given that, “dual function” refers to a protein that is capable of at least two activities when expressed, and “multi-function” refers to a protein that is capable of more than one activity when expressed.

“Block” or “inhibit” has their ordinary meaning as understood in light of the specification, and refer to reducing or alleviating the functional activity of a protein. For example, if a protein is capable of activating a cellular signal, then blocking that function reduces or eliminates the activation of that cellular signal.

“Specific” or “Specificity” has its ordinary meaning as understood in light of the specification, and can refer to the characteristic of a ligand for the binding partner or alternatively, the binding partner for the ligand, and can include complementary shape, charge, and hydrophobic specificity for binding. Specificity for binding can include stereospecificity, regioselectivity and chemoselectivity. In some embodiments, a chimeric antigen receptor is provided, wherein the chimeric antigen receptor is specific for a B-cell ligand. In some embodiments, a chimeric antigen receptor is provided, wherein the chimeric antigen receptor is specific for a tumor cell ligand.

“Constitutive” has its ordinary meaning as understood in light of the specification, and can refer to the characteristic of an activity that does not need to be induced, or is considered “always on” in a cell, tissue, organ, or system. For example, a “constitutive gene” refers to a gene that is expressed continuously in a cell, and a “constitutive signal” refers to a signal in a cell, tissue, or system, that is continuously active.

“Downstream” and “upstream” have their ordinary meaning as understood in light of the specification, and refer to the relation of one entity compared to another. In the case of a sequence, for any given nucleotide or amino acid “N,” an upstream nucleotide or amino acid occurs previously in that sequence. In contrast, a downstream nucleotide or amino acid occurs later in that sequence. In the case of a cellular pathway or function, for event “A,” an upstream event occurs before event “A,” while a downstream event occurs after event “A.” It will therefore be understood that “downstream signaling,” as used in the present specification, refers to signaling that occurs after the function of the referenced protein occurs.

A “regulatory element” has its ordinary meaning as understood in light of the specification, and as described herein, can refer to a regulatory sequence, which is any DNA sequence that is responsible for the regulation of gene expression, such as promoters and operators. The regulatory element can be a segment of a nucleic acid molecule which is capable of increasing or decreasing the expression of specific genes within an organism. In some embodiments described herein, a cell is provided, wherein the cell includes a first and second chimeric antigen receptor or TcR, wherein the first chimeric antigen receptor is specific for a ligand on a B cell, which promotes the in vivo expansion and activation of an effector cell and, wherein the second chimeric antigen receptor or TcR is specific for a ligand on a tumor. In some embodiments, the first chimeric antigen receptor and/or the second chimeric antigen receptor or TcR are inducibly expressed in said cell. In some embodiments, expression of the first chimeric antigen receptor and/or the second chimeric antigen receptor or TcR is under the control of a regulatory element.

“Transmembrane domain” has its ordinary meaning as understood in light of the specification, and as described herein is an integral protein that can span a cellular membrane.

A “promoter” has its ordinary meaning as understood in light of the specification, and is a nucleotide sequence that directs the transcription of a structural gene. In some embodiments, a promoter is located in the 5′ non-coding region of a gene, proximal to the transcriptional start site of a structural gene. Sequence elements within promoters that function in the initiation of transcription are often characterized by consensus nucleotide sequences. Without being limiting, these promoter elements can include RNA polymerase binding sites, TATA sequences, CAAT sequences, differentiation-specific elements (DSEs; McGehee et al., Mol. Endocrinol. 7:551 (1993); incorporated by reference in its entirety), cyclic AMP response elements (CREs), serum response elements (SREs; Treisman, Seminars in Cancer Biol. 1:47 (1990); incorporated by reference in its entirety), glucocorticoid response elements (GREs), and binding sites for other transcription factors, such as CRE/ATF (O'Reilly et al., J. Biol. Chem. 267:19938 (1992); incorporated by reference in its entirety), AP2 (Ye et al., J. Biol. Chem. 269:25728 (1994); incorporated by reference in its entirety), SP1, cAMP response element binding protein (CREB; Loeken, Gene Expr. 3:253 (1993); incorporated by reference in its entirety) and octamer factors (see, in general, Watson et al., eds., Molecular Biology of the Gene, 4th ed. (The Benjamin/Cummings Publishing Company, Inc. 1987; incorporated by reference in its entirety), and Lemaigre and Rousseau, Biochem. J. 303:1 (1994); incorporated by reference in its entirety). As used herein, a promoter can be constitutively active, repressible, or inducible. If a promoter is an inducible promoter, then the rate of transcription increases in response to an inducing agent. In contrast, the rate of transcription is not regulated by an inducing agent if the promoter is a constitutive promoter. Repressible promoters are also known. In some embodiments of the nucleic acid, the nucleic acid includes a promoter sequence. In some embodiments of the chimeric antigen, the chimeric antigen is inducibly expressed in response to an inducing agent. In some embodiments, the TcR is inducibly expressed in response to an inducing agent.

In some embodiments, promoters used herein can be inducible or constitutive promoters. Without being limiting, inducible promoters can include, for example, a tamoxifen inducible promoter, tetracycline inducible promoter, and doxocycline inducible promoter (e.g., tre) promoter. Constitutive promoters can include, for example, SV40, CMV, UBC, EF1alpha, PGK, and CAGG. In some embodiments, the regulatory element is a promoter. In some embodiments, the promoter is a tamoxifen inducible promoter, a tetracycline inducible promoter, or a doxocycline inducible promoter (e.g., tre) promoter. In some embodiments provided herein, expression of a chimeric antigen receptor or a TcR on a cell is induced by tamoxifen and/or its metabolites. Metabolites for tamoxifen are active metabolites such as 4-hyroxytamoxifen (afimoxifene) and N-desmethyl-4-hydroxytamoxifen (endoxifen), which can have 30-100 times more affinity with an estrogen receptor than tamoxifen itself. In some embodiments, the tamoxifen metabolites are 4-hyroxytamoxifen (afimoxifene) and/or N-desmethyl-4-hydroxytamoxifen (endoxifen). In some embodiments, vectors are provided wherein the vector has a first promoter for the CAR/TcR and a second promoter for the marker protein.

The term “cell” includes those of prokaryotes and eukaryotes, and may further include bacterial cells, mycobacteria cells, fungal cells, yeast cells, plant cells, insect cells, non-human animal cells, human cells, or cell fusions such as, for example, hybridomas. In some embodiments, the cell is eukaryotic. In some embodiments, the cell is a mammalian cell. In some embodiments, the cell is derived from human, monkey, ape, hamster, rat, or mouse cells. In some embodiments, the cell is human. In some embodiments, the cell is an immune cell. In some embodiments, the cell is a lymphocyte. In some embodiments, the cell is a T cell. In some embodiments, the cell is a tumor infiltrating lymphocyte (TIL) cell, a natural killer (NK) cell, a CD8+ T cell, a CD4+ T cell, a regulatory T cell, or a memory T cell.

An “antibody” as described herein, has its ordinary meaning as understood in light of the specification, and refers to a large Y-shape protein produced by plasma cells that is used by the immune system to identify and neutralize foreign objects such as bacteria and viruses. The antibody protein can include four polypeptide chains; two identical heavy chains and two identical light chains connected by disulfide bonds. Each chain is composed of structural domains called immunoglobulin domains. These domains can contain about 70, 80, 90, 100, 110, 120, 130, 140, 150 amino acids or any number of amino acids in between in a range defined by any two of these values, and are classified into different categories according to their size and function. In some embodiments, the ligand binding domain includes an antibody or binding fragment thereof or scFv, a receptor ligand or mutants thereof, peptide, and/or polypeptide affinity molecule or binding partner. In some embodiments, the ligand binding domain is an antibody fragment, desirably, a binding portion thereof. In some embodiments, the antibody fragment or binding portion thereof present on a CAR is specific for a ligand on a B-cell. In some embodiments, the antibody fragment or binding portion thereof present on a CAR or TcR is specific for a ligand on a tumor cell. In some embodiments, the tumor is not derived from a B-cell related cancer. In some embodiments, the antibody fragment or binding portion thereof present on a CAR is specific for a ligand present on a tumor cell. In some embodiments, the ligand binding domain is an antibody fragment or a binding portion thereof, such as a single chain variable fragment (scFv). In some embodiments, the ligand includes a tumor specific mutation. In some embodiments, the antibody fragment or binding portion thereof present on a CAR includes one or more domains from a humanized antibody, or binding portion thereof.

An “antigen” as described herein, has its ordinary meaning as understood in light of the specification, and refers to any molecule capable of inducing an immune response in a subject. In some embodiments, the antigen binds to an at least one antibody.

Specific binding of an antibody to its target antigen(s) means an affinity of at least 10⁶, 10⁷, 10⁸, 10⁹, or 10¹⁰ M⁻¹. Specific binding is detectably higher in magnitude and distinguishable from non-specific binding occurring to at least one unrelated target. Specific binding can be the result of formation of bonds between particular functional groups or particular spatial fit (e.g., lock and key type) whereas nonspecific binding is usually the result of van der Waals forces. Specific binding does not however necessarily imply that an antibody or fusion protein binds one and only one target.

A basic antibody structural unit is a tetramer of subunits. Each tetramer includes two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. This variable region is initially expressed linked to a cleavable signal peptide. The variable region without the signal peptide is sometimes referred to as a mature variable region. Thus, for example, a light chain mature variable region means a light chain variable region without the light chain signal peptide. However, reference to a variable region does not mean that a signal sequence is necessarily present; and in fact, signal sequences are cleaved once the antibodies or fusion proteins have been expressed and secreted. A pair of heavy and light chain variable regions defines a binding region of an antibody. The carboxy-terminal portion of the light and heavy chains respectively defines light and heavy chain constant regions. The heavy chain constant region is primarily responsible for effector function. In IgG antibodies, the heavy chain constant region is divided into CH1, hinge, CH2, and CH3 regions. The CH1 region binds to the light chain constant region by disulfide and noncovalent bonding. The hinge region provides flexibility between the binding and effector regions of an antibody and also provides sites for intermolecular disulfide bonding between the two heavy chain constant regions in a tetramer subunit. The CH2 and CH3 regions are the primary site of effector functions and FcR binding.

Light chains are classified as either kappa or lambda. Heavy chains are classified as gamma, mu, alpha, delta, or epsilon, and define the antibody's isotype as IgG, IgM, IgA, IgD and IgE, respectively. Within light and heavy chains, the variable and constant regions are joined by a “J” segment of about 12 or more amino acids, with the heavy chain also including a “D” segment of about 10 or more amino acids. (See generally, Fundamental Immunology (Paul, W., ed., 2^{nd} ed. Raven Press, N.Y., 1989), Ch. 7) (incorporated by reference in its entirety for all purposes).

The IgG antibodies include Fc and Fab domains. The Fab fragment can further be divided into the Fv fragment, which includes a heavy and light chain, and is the smallest fragment that still retains the antigen binding site.

The mature variable regions of each light/heavy chain pair form the antibody binding site. Thus, an intact antibody has two binding sites, for example, is divalent. In natural antibodies, the binding sites are the same. However, bispecific antibodies can be made in which the two binding sites are different (see, e.g., Songsivilai S, Lachmann P C. 1990. Bispecific antibody: a tool for diagnosis and treatment of disease. Clin Exp Immunol. 79:315-321; Kostelny S A, Cole M S, Tso J Y. 1992. Formation of bispecific antibody by the use of leucine zippers. J Immunol. 148: 1547-1553). The variable regions all exhibit the same general structure of relatively conserved framework regions (FR) joined by three hypervariable regions, also called complementarity determining regions or CDRs. The CDRs from the two chains of each pair are aligned by the framework regions, enabling binding to a specific epitope. From N-terminal to C-terminal, both light and heavy chains include the domains FR1, CDR1, FR2, CDR2, FR3, CDR3 and FR4. For convenience, the variable heavy CDRs can be referred to as CDR_{H}1, CDR_{H}2 and CDR_{H}3; the variable light chain CDRs can be referred to as CDR_{L}1, CDR_{L}2 and CDR_{L}3. The assignment of amino acids to each domain is in accordance with the definitions of Kabat E A, et al. 1987 and 1991. Sequences of Proteins of Immunological Interest (National Institutes of Health, Bethesda, MD) or Chothia C, Lesk A M. 1987. Canonical Structures for the Hypervariable Regions of Immunoglobulins. J Mol Biol 196:901-917; Chothia C, et al. 1989. Conformations of Immunoglobulin Hypervariable Regions. Nature 342:877-883. Kabat also provides a widely used numbering convention (Kabat numbering) in which corresponding residues between different heavy chain variable regions or between different light chain variable regions are assigned the same number. Although Kabat numbering can be used for antibody constant regions, EU numbering is more commonly used, as is the case in this application. Although specific sequences are provided for exemplary antibodies disclosed herein, it will be appreciated that after expression of protein chains one to several amino acids at the amino or carboxy terminus of the light and/or heavy chain, particularly a heavy chain C-terminal lysine residue, may be missing or derivatized in a proportion or all of the molecules.

In addition to entire immunoglobulins (or their recombinant counterparts), immunoglobulin fragments or “binding fragments” including the epitope binding site (e.g., Fab′, F(ab′)₂, single-chain variable fragment (scFv), diabody, minibody, nanobody, single-domain antibody (sdAb), or other fragments) are useful as antibody moieties in the present disclosure. Such antibody fragments may be generated from whole immunoglobulins by ricin, pepsin, papain, or other protease cleavage. Minimal immunoglobulins may be designed utilizing recombinant immunoglobulin techniques. For instance, “Fv” immunoglobulins for use in the present disclosure may be produced by linking a variable light chain region to a variable heavy chain region via a peptide linker (e.g., poly-glycine or another sequence which does not form an alpha helix or beta sheet motif). Nanobodies or single-domain antibodies can also be derived from alternative organisms, such as dromedaries, camels, llamas, alpacas, or sharks. In some embodiments, antibodies can be conjugates, e.g., pegylated antibodies, drug, radioisotope, or toxin conjugates. Monoclonal antibodies directed against a specific epitope, or combination of epitopes, will allow for the targeting and/or depletion of cellular populations expressing the marker. Various techniques can be utilized using monoclonal antibodies to screen for cellular populations expressing the marker(s), and include magnetic separation using antibody-coated magnetic beads, “panning” with antibody attached to a solid matrix (for example, a plate), and flow cytometry.

As known in the art, the term “Fc region” is used to define a C-terminal region of an immunoglobulin heavy chain. The “Fc region” may be a native sequence Fc region or a variant Fc region. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The numbering of the residues in the Fc region is that of the EU index as in Kabat. Kabat et al., Sequences of Proteins of Immunological Interest, 5th Ed. Public Health Service, National Institutes of Health, Bethesda, Md., 1991. The Fc region of an immunoglobulin generally includes two constant domains, CH2 and CH3. As is known in the art, an Fc region can be present in dimer or monomeric form.

As known in the art, a “constant region” of an antibody refers to the constant region of the antibody light chain or the constant region of the antibody heavy chain, either alone or in combination.

A “variable region” of an antibody refers to the variable region of the antibody light chain or the variable region of the antibody heavy chain, either alone or in combination. As known in the art, the variable regions of the heavy and light chains each consist of four framework regions (FRs) connected by three complementarity determining regions (CDRs) also known as hypervariable regions, and contribute to the formation of the antigen binding site of antibodies. If variants of a subject variable region are desired, particularly with substitution in amino acid residues outside of a CDR region (for example, in the framework region), appropriate amino acid substitution, preferably, conservative amino acid substitution, can be identified by comparing the subject variable region to the variable regions of other antibodies which contain CDR1 and CDR2 sequences in the same canonical class as the subject variable region (Chothia and Lesk, J Mol Biol 196(4): 901-917, 1987).

The term “epitope” refers to a site on an antigen to which an antibody or extracellular trap segment binds. An epitope on a protein can be formed from contiguous amino acids or noncontiguous amino acids juxtaposed by tertiary folding of one or more proteins. Epitopes formed from contiguous amino acids (also known as linear epitopes) are typically retained on exposure to denaturing solvents whereas epitopes formed by tertiary folding (also known as conformational epitopes) are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols, in Methods in Molecular Biology, Vol. 66, Glenn E. Morris, Ed. (1996).

Antibodies that recognize the same or overlapping epitopes can be identified in a simple immunoassay showing the ability of one antibody to compete with the binding of another antibody to a target antigen. The epitope of an antibody can also be defined by X-ray crystallography of the antibody (or Fab fragment) bound to its antigen to identify contact residues.

Alternatively, two antibodies have the same epitope if all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

Competition between antibodies is determined by an assay in which an antibody under test inhibits specific binding of a reference antibody to a common antigen (see, e.g., Junghans et al., Cancer Res. 50: 1495, 1990). A test antibody competes with a reference antibody if an excess of a test antibody (e.g., at least 2×, 5×, 10×, 20×, or 100×) inhibits binding of the reference antibody by at least 50%. In some embodiments the test antibody inhibits binding of the reference antibody by 75%, 90%, or 99% as measured in a competitive binding assay. Antibodies identified by competition assay (competing antibodies) include antibodies binding to the same epitope as the reference antibody and antibodies binding to an adjacent epitope sufficiently proximal to the epitope bound by the reference antibody for steric hindrance to occur.

“Immune cells” as described herein, have their ordinary meaning as understood in light of the specification, and refer to cells that are part of the immune system. In some embodiments, the cell is part of the innate immune system. In some embodiments, the cell is part of the adaptive immune system. Non-limiting example of immune cells include blood cells, bone marrow cells, hematopoietic stem cells, lymphoid progenitor cells, myeloid progenitor cells, B cell progenitors, memory B cells, plasma cells, monocytes, macrophages, dendritic cells, basophils, neutrophils, eosinophils, mast cells, natural killer cells, T cell progenitors, memory T cell, cytotoxic T cells, and helper T cells.

“Effector cells” as described herein, has its ordinary meaning as understood in light of the specification, and refers to a lymphocyte that has been induced to differentiate into another cell type that can be capable of mounting a specific immune response, such as a terminally differentiated leukocyte that performs one or more specific functions. The main effector cells of the immune system, for example, are activated lymphocytes and phagocytes that are involved in destroying pathogens and removing them from the body. The effector cells can include large granular lymphocytes, such as, for example, natural killer cells and cytotoxic T lymphocytes. In some embodiments of the cells provided herein, the cell includes a first and second chimeric antigen receptor, wherein the first chimeric antigen receptor is specific for a ligand on a B cell, which promotes the in vivo expansion and activation of an effector cell and, wherein the second chimeric antigen receptor is specific for a ligand on a tumor. In some embodiments, the cells that undergo expansion and activation are lymphocytes, phagocytes, large granular lymphocytes, natural killer cells and/or cytotoxic T lymphocytes.

“Cancer antigen,” “tumor antigen,” or “tumor marker” has its ordinary meaning as understood in light of the specification, and refers to an antigenic substance that is produced in a tumor cell, which can therefore trigger an immune response in the host. These cancer antigens can be useful as markers for identifying a tumor cell, which will be a potential candidate during treatment or therapy. There are several types of cancer or tumor antigens. There are tumor specific antigens (TSA) which are present only on tumor cells and not on healthy cells, as well as tumor associated antigens (TAA) which are present in tumor cells and also on some normal cells. In some embodiments of the methods and chimeric antigens provided herein, the chimeric antigen receptors are specific for tumor specific antigens. In some embodiments, the chimeric antigen receptors are specific for tumor associated antigens. In some embodiments described herein, the tumor does not originate from a B-cell related cancer. In some embodiments, cells expressing a CAR that is specific for a TAA is further modified by the introduction of a suicide gene to limit the time of the CAR T-cell therapy and to reduce the attack of normal tissues expressing the TAA.

In any of the embodiments provided herein, the cancer antigen is EGFR, HER2, Mesothelin, cancer testis antigens, L1CAM, o-acetylated GD2, GD2, neoantigens, Var2, glypican-2 (GPC2), HPV antigens, alphafetoprotein, carcinoembryonic antigen, CA-125, MUC-1, epithelial tumor antigen, abnormal products of ras or p53, EphA2, MAGE-A3, MAGE-A4, MAGE-C2, PRAME, SSX2, adipophilin, AIM2, ALDH1A1, BCLX, B7H3, EpCAM, claudin 18.2, CS274, CPSF, cyclin D1, DKK1, ENAH, EpCAM, EphA3, EZH2, FAP, FGF5, glypican-3, G250, HLA-DOB, Hepsin, ID01, IGF2B3, IL13Ralpha2, Intestinal carboxylesterase, alpha-foetoprotein, kallikrein4, KIF20A, Lengsin, M-CSF, MCSP, mdm-2, Meloe, midkine, MMP-2, MMP-7, MUC1, MUC5AC, p53, PAX5, PBF, PRAME, PSMA, RAGE-1, RGS5, RhoC, RNF43, RUF43, FU2AS, secernin 1, SOX10, STEAP1, survivin, telomerase, TPBG, VEGF, WT1, NY-ESO-1, or ROR1. In some embodiments, the cell surface tumor specific molecule is ROR1. In some embodiments, the cancer antigen is expressed by a tumor. In some embodiments, the tumor is not a B-cell related cancer.

“Chimeric antigen receptors” (CARs), as described herein, has its ordinary meaning as understood in light of the specification, and refers to genetically engineered protein receptors, which can confer specificity onto an immune effector cell, such as for example, a T-cell. Without being limiting, the use of CAR bearing T-cells can promote in vivo expansion and activation. The CARs can also be designed to redirect T-cells to target cells that express specific cell-surface antigens, where they can activate lymphocytes, such as T-cells, upon target recognition. The CARs graft the specificity of a monoclonal antibody or binding fragment thereof or scFv onto a T-cell, with the transfer of their coding sequence facilitated by vectors. In order to use CARs as a therapy for a subject in need, a technique called adoptive cell transfer is used in which T-cells are removed from a subject and modified so that they can express the CARs that are specific for an antigen. The T-cells, which can then recognize and target an antigen, are reintroduced into the patient. In some embodiments, CAR expressing lymphocytes are described, wherein the CAR expressing lymphocyte can be delivered to a subject to target specific cells. A TcR is a molecule on the surface of T lymphocytes or T-cells that can recognize antigens. As described herein, the CAR promotes in vivo expansion and activation of effector cells.

The structure of the CAR can include fusions of single-chain variable fragments (scFv) that are derived from monoclonal antibodies that are attached to transmembrane and cytoplasmic signaling domains. Most CARs can include an extracellular scFv that is linked to an intracellular CD3ζ domain (first generation CAR). Additionally, the scFv can be linked to a co-stimulatory domain, which can increase their efficacy in the therapy of a subject in need (second generation CAR). When T-cells express this molecule, they can recognize and kill target cells that express a specific antigen targeted by the CAR.

The chimeric antigen receptor can include a binding portion that is specific for a ligand. Without being limiting, the binding portion can include an antibody or binding fragment thereof or scFv, a receptor ligand or mutants thereof, peptide, and/or polypeptide affinity molecule or binding partner. In some embodiments of the first chimeric antigen receptor, the first chimeric antigen receptor includes a binding portion, wherein the binding portion includes an antibody or binding fragment thereof or scFv, a receptor ligand or mutants thereof, peptide, and/or polypeptide affinity molecule or binding partner. In some embodiments, the binding portion is specific for a ligand on a B-cell. In some embodiments of the second chimeric antigen receptor, the second chimeric antigen receptor includes a binding portion, wherein the binding portion includes an antibody or binding fragment thereof or scFv, a receptor ligand or mutants thereof, peptide, and/or polypeptide affinity molecule or binding partner. In some embodiments, the binding portion is specific for a ligand on a tumor cell. In some embodiments, the tumor is not a tumor of a B-cell related cancer.

In some embodiments, a chimeric antigen receptor is provided, wherein the ligand or target molecule is a cell surface molecule that is found on tumor cells and is not substantially found on normal tissues, or restricted in its expression to non-vital normal tissues. In some embodiments, the tumor does not originate from a B-cell related cancer. In some embodiments, the ligand or target molecule is found on a tumor cell as well as on normal tissues. In some embodiments, the cells expressing a CAR that is specific for a ligand on tumor cells and normal tissue further includes a suicide gene to limit the time of therapy and increase their safety profile. Conditional suicide genes may also be applied to the donor T-cells to limit the attack on normal tissue that may express a tumor associated antigen or ligand.

Although humanized antibodies often incorporate all six CDRs (which can be as defined by Kabat) from a mouse antibody, they can also be made with less than all CDRs (e.g., at least 3, 4, or 5 CDRs from a mouse antibody) (e.g., De Pascalis R, Iwahashi M, Tamura M, et al. 2002. Grafting “Abbreviated” Complementary-Determining Regions Containing Specificity-Determining Residues Essential for Ligand Contact to Engineer a Less Immunogenic Humanized Monoclonal Antibody. J Immunol. 169:3076-3084; Vajdos F F, Adams C W, Breece T N, Presta L G, de Vos A M, Sidhu, S S. 2002. Comprehensive functional maps of the antigen-binding site of an anti-ErbB2 antibody obtained with shotgun scanning mutagenesis. J Mol Biol. 320: 415-428; Iwahashi M, Milenic D E, Padlan E A, et al. 1999. CDR substitutions of a humanized monoclonal antibody (CC49): Contributions of individual CDRs to antigen binding and immunogenicity. Mol Immunol. 36:1079-1091; Tamura M, Milenic D E, Iwahashi M, et al. 2000. Structural correlates of an anticarcinoma antibody: Identification of specificity-determining regions (SDRs) and development of a minimally immunogenic antibody variant by retention of SDRs only. J Immunol. 164:1432-1441).

A chimeric antibody is an antibody in which the mature variable regions of light and heavy chains of a non-human antibody (e.g., a mouse) are combined with human light and heavy chain constant regions. Such antibodies substantially or entirely retain the binding specificity of the mouse antibody, and are about two-thirds human sequence.

“Signaling domain” as described herein, has its ordinary meaning as understood in light of the specification, and is a domain on a chimeric antigen receptor that can promote cytokine release, in vivo T cell survival and tumor elimination. In some embodiments herein, a signaling domain includes CD28, 4-1BB, and/or CD3-zeta cytoplasmic domains.

A “cytokine” as described herein, has its ordinary meaning as understood in light of the specification, and is a small molecule that is secreted by one cell and that has an effect on other cells. Cytokines, sometime considered as “stress proteins,” include chemokines, interferons, interleukins, lymphokines, and tumor necrosis factors. Cytokines are produced by many cells, including macrophages, B lymphocytes, T lymphocytes and mast cells, as well as endothelial cells, fibroblasts, and various stromal cells. A particular cytokine may be produced by more than one type of cell. Non-limiting examples of cytokines include members of the IL-1 family, TNF family, interferons, IL-6 family, IL-10 family, TGF-beta family, and chemokines. Common cytokines include IL-1, IL-2, IL-3, IL-4, IL-5, IL-6, IL-8, IL-9, IL-10, IL-11, IL-12, IL-13, IL-15, IL-16, IL-18, IL-21, IL-33, IFN-alpha, IFN-beta, IFN-gamma, TNF-alpha, and TNF-beta.

“Cytokine signaling” as described herein, has its ordinary meaning as understood in light of the specification, and refers to the process by which a cytokine is recognized by a cytokine receptor on the surface of a cell, and elicits a response. These signals may either be “autocrine” (wherein the same cell both produces the cytokine and responds to it) or “paracrine” (where the cytokine is made by one cell and acts on another). Cytokine receptors are grouped into six major families: class I cytokine receptors, class II cytokine receptors, IL-1 receptors, TNF receptors, tyrosine kinase receptors, and chemokine receptors. Cytokines activate many pathways; for example, the JAK-STAT pathway. In this pathway, JAK proteins phosphorylate a cytokine receptor once that receptor binds to its corresponding cytokine. This newly phosphorylated residue on the cytokine receptor then acts as a binding site for a STAT protein. Once the STAT is bound, it is phosphorylated by JAK and forms a homodimer with another STAT. This complex then dissociates from the receptor, travels to the nucleus, and induces transcription of crucial genes.

### Methods of Treatment

Some embodiments relate to methods of treatment as part of immunotherapy. “Immunotherapy” has its ordinary meaning as understood in light of the specification, and refers to the process of using a subject's immune system to fight a disease. In some embodiments, immunotherapy is used to target a cancer. In some embodiments, the immunotherapy includes activating an immunoinhibitory pathway. “Immunoinhibitory pathway” has its ordinary meaning as understood in light of the specification, and refers to a pathway that inhibits, reduces, or eliminates immune function.

“Solid Tumors” as described herein, has its ordinary meaning as understood in light of the specification, and refers to a malignant cancerous mass of tissue. In some embodiments of the methods of treating, ameliorating, or inhibiting a non-B cell related disease in a subject provided herein, the method includes introducing, providing, or administering any one or more of the cells or compositions of any of the embodiments herein or the cells made by any one or more of the methods of the embodiments herein into a subject for therapy. In some embodiments, the subject has a cancer. In some embodiments, the cancer is a solid tumor. In some embodiments, the solid tumor is a breast cancer, brain cancer, lung cancer, liver cancer, stomach cancer, spleen cancer, colon cancer, renal cancer, pancreatic cancer, prostate cancer, uterine cancer, skin cancer, head cancer, neck cancer, sarcomas, neuroblastomas, or ovarian cancer.

“Engraftment” as described herein, has its ordinary meaning as understood in light of the specification, and refers to the incorporation of grafted tissue into the body of the host. Several characteristics of effective CAR T-cells include showing signs of adequate engraftment, which is required for responses. For example, detection of the CAR transgene by polymerase chain reaction is not informative about the surface expression of the CAR, which is the only form that matters for efficacy. Thus, the availability of reagents to specifically detect CARs at the cell surface by flow cytometry or other methods known to those skilled in the art is crucial to understand the activity and engraftment of CAR T-cells. In the embodiments described herein, the therapeutic potency of the adoptively transferred CARs are improved by allowing a B-cell targeting CAR to drive the activation, proliferation and dispersion of infused CAR T-cells that have a second CAR that provides for redirected killing of the solid tumor. In some embodiments described herein, the methods and cells including a CAR with B-cell specificity led to the surprising effect of having an improved level of engraftment compared to T-cells that only included CARs specific for a tumor ligand. As described in the embodiments herein, the obstacle of failure to exhibit engraftment is overcome by allowing a B cell targeting CAR to drive the activation, proliferation and dispersion of infused CAR T-cells that have a CAR that provides for redirected killing of the solid tumor.

“Subject” or “patient,” as described herein, has its ordinary meaning as understood in light of the specification, and refers to any organism upon which the embodiments described herein may be used or administered, e.g., for experimental, diagnostic, prophylactic, and/or therapeutic purposes. Subjects or patients include, for example, animals. In some embodiments, the subject is mice, rats, rabbits, non-human primates, and humans. In some embodiments, the subject is a cow, sheep, pig, horse, dog, cat, primate, or a human.

As used herein, the terms “treat,” “treating,” “treated,” or “treatment” has its ordinary meaning as understood in light of the specification, and refer to both therapeutic treatment and prophylactic or preventative treatment.

As used herein, the terms “ameliorate,” “ameliorating,” “amelioration,” or “ameliorated” has its ordinary meaning as understood in light of the specification, and in reference to cancer can mean reducing the symptoms of the cancer, reducing the size of a tumor, completely or partially removing the tumor (e.g., a complete or partial response), causing stable disease, preventing progression of the cancer (e.g., progression free survival), or any other effect on the cancer that would be considered by a physician to be a therapeutic, prophylactic, or preventative treatment of the cancer.

As used herein, the terms “administer,” administering,” or “administered” has its ordinary meaning as understood in light of the specification, and includes all means of introducing the compound, or pharmaceutically acceptable salt thereof, or CAR T cell composition, wherein the CAR T cell composition includes CAR T cells and wherein the CAR includes an E2 anti-fluorescein antibody fragment, to the patient, including, but not limited to, oral, intravenous, intratumoral, intramuscular, subcutaneous, and transdermal.

As used herein, the terms “transduction” and “transfection” has its ordinary meaning as understood in light of the specification, and are used equivalently and the terms mean introducing a nucleic acid into a cell by any artificial method, including viral and non-viral methods.

An “effective amount” of an agent, e.g., a pharmaceutical formulation, agent, cells, or composition, in the context of administration, refers to an amount effective, at dosages/amounts and for periods of time necessary, to achieve a desired result, such as a therapeutic or prophylactic result.

A “therapeutically effective amount” of a composition, e.g., a pharmaceutical formulation including agents or cells, refers to an amount effective, at dosages and for periods of time necessary, to achieve a desired therapeutic result, such as for treatment of a disease, condition, or disorder, and/or pharmacokinetic or pharmacodynamic effect of the treatment. The therapeutically effective amount may vary according to factors such as the disease state, age, sex, and weight of the subject, and the agents or populations of cells administered. In some embodiments, the provided methods involve administering the agents, cells and/or compositions at effective amounts, e.g., therapeutically effective amounts.

As used herein, the term “standard of care”, “best practice” and “standard therapy” refers to the treatment that is accepted by medical practitioners to be an appropriate, proper, effective, and/or widely used treatment for a certain disease. The standard of care of a certain disease depends on many different factors, including the biological effect of treatment, region or location within the body, patient status (e.g. age, weight, gender, hereditary risks, other disabilities, secondary conditions), toxicity, metabolism, bioaccumulation, therapeutic index, dosage, and other factors known in the art. Determining a standard of care for a disease is also dependent on establishing safety and efficacy in clinical trials as standardized by regulatory bodies such as the US Food and Drug Administration, International Council for Harmonisation, Health Canada, European Medicines Agency, Therapeutics Goods Administration, Central Drugs Standard Control Organization, National Medical Products Administration, Pharmaceuticals and Medical Devices Agency, Ministry of Food and Drug Safety, and the World Health Organization. The standard of care for a disease may include but is not limited to surgery, radiation, chemotherapy, targeted therapy, or immunotherapy (e.g., PD1/PDL1 or CTLA4 blockade therapy).

The above description discloses several methods and materials of the embodiments described herein. Some embodiments provided herein are susceptible to modifications in the methods and materials, as well as alterations in the fabrication methods and equipment. Such modifications will become apparent to those skilled in the art from a consideration of this disclosure or practice of the embodiments disclosed herein. Consequently, it is not intended that this disclosure be limited to the specific embodiments disclosed herein, but that it covers all modifications and alternatives coming within the true scope and spirit of the disclosure.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

In another embodiment of the methods described herein, any of the methods described herein can be used alone, or any of the methods described herein can be used in combination with any other method or methods described herein.

### EXAMPLES

While the present disclosure has been described in some detail for purposes of clarity and understanding, one skilled in the art will appreciate that various changes in form and detail can be made without departing from the true scope of the embodiments described herein.

### Example 1: Production and Purification of Multi-Specific Reagents

Sixteen constructs were generated, as outlined in the below Table 1. The sequences of the full constructs, and the construct domains, are as outlined in Tables 3-6.

TABLE 1
Domains of Multi-Specific Reagent Constructs

| Construct: | Domain Composition (from N to C Terminus): |
|---|---|
| CM04A | anti-PEG _ anti-CD3 |
| CM04B | mutated ApoE3 LBD (lipid binding domain only) _ anti-CD3 |
| CM04C | anti-PEG _ mutated ApoE3 LBD _ anti-CD3 |
| CM04D | mutated ApoE3 FL (full length protein) _ anti-CD3 |
| CM04E | anti-PEG _ mutated ApoE3 FL _ anti-CD3 |
| CM04F | anti-PEG _ anti-TRBC1 (TCR-β1) |
| CM04G | mutated ApoE3 LBD _ anti-TRBC1 |
| CM04H | anti-PEG _ mutated ApoE3 LBD _ anti- TRBC1 |
| CM04I | mutated ApoE3 FL _ anti- TRBC1 |
| CM04J | anti-PEG _ mutated ApoE3 FL _ anti- TRBC1 |
| CM04K | anti-PEG _ anti-CD5 |
| CM04L | mutated ApoE3 LBD _anti-CD5 |
| CM04M | anti-PEG _ mutated ApoE3 LBD _ anti-CD5 |
| CM04N | mutated ApoE3 FL _ anti-CD5 |
| CM04O | anti-PEG _ mutated ApoE3 FL _ anti-CD5 |
| CM04P | anti-PEG _ anti-EpCAM |

TABLE 2
Amino Acid Sequences of Linkers, Tags, and Signal Peptides

| Linker and Tag Name: | SEQ ID NO: | Sequence: |
|---|---|---|
| Linker 1 | 1 | GGGGSGGGGSGGGGS |
| Linker 2 | 2 | GGSSRSSSSGGGGSGGGG |
| Linker 3 | 3 | SPNSASHSGSAPQTSSAPGSQ |
| Linker 4 | 4 | EPKSSDKTHTSPPSPAPEL |
| HIS tag | 5 | GGGGSHHHHHHHH |
| IL-2 signal peptide | 53 | MYRMQLLSCIALSLALVTNS |

TABLE 3
Amino Acid Sequences of Multi-Specific Reagent Constructs

| | SEQ ID | |
|---|---|---|
| Construct: | NO: | Sequence: |
| CM04A | 6 | QIQLVQSGPELKKPGETVKISCKASGYTFKNYGMNWVKQAPGKGLKW |
| | | MGWINTYTGQPIYANDFKGRFAFSLETSASTAYLQINNLKNEDTATYFC |
| | | ARDWGPYWGQGTLVIVSASPNSASHSGSAPQTSSAPGSQNIMMTQSPSS |
| | | LAVSAGEKVTVNCKSSQSVLYSSNQMNYLAWYQQKPGQSPKLLIYWAS |
| | | TRESGVPDRFTGSGSGTDFTLTISSVQTEDLAVYYCLQYLSSWTFGGGTK |
| | | LEIKGGSSRSSSSGGGGSGGGGEVQLLESGGGLVQPGGSLRLSCAASGFT |
| | | FSSFPMAWVRQAPGKGLEWVSTISTSGGRTYYRDSVKGRFTISRDNSKN |
| | | TLYLQMNSLRAEDTAVYYCAKFRQYSGGFDYWGQGTLVTVSSSPNSAS |
| | | HSGSAPQTSSAPGSQDIQLTQPNSVSTSLGSTVKLSCTLSSGNIENNYVHW |
| | | YQLYEGRSPTTMIYDDDKRPDGVPDRFSGSIDRSSNSAFLTIHNVAIEDEA |
| | | IYFCHSYVSSFNVFGGGTKLTVLRGGGGSHHHHHHHH |
| CM04B | 7 | AGQPLQERAQAWGERLRARMEEMGSRTRDRLDEVKEQVAEVRAKLEE |
| | | QAQQIRLQAEAAQARLKSRFEPLAEDMQRQWAGQVEKVQAAEGTSAAP |
| | | VPSDNHGGSSRSSSSGGGGSGGGGEVQLLESGGGLVQPGGSLRLSCAAS |
| | | GFTFSSFPMAWVRQAPGKGLEWVSTISTSGGRTYYRDSVKGRFTISRDNS |
| | | KNTLYLQMNSLRAEDTAVYYCAKFRQYSGGFDYWGQGTLVTVSSSPNS |
| | | ASHSGSAPQTSSAPGSQDIQLTQPNSVSTSLGSTVKLSCTLSSGNIENNYV |
| | | HWYQLYEGRSPTTMIYDDDKRPDGVPDRFSGSIDRSSNSAFLTIHNVAIE |
| | | DEAIYFCHSYVSSFNVFGGGTKLTVLRGGGGSHHHHHHHH |
| CM04C | 8 | QIQLVQSGPELKKPGETVKISCKASGYTFKNYGMNWVKQAPGKGLKW |
| | | MGWINTYTGQPIYANDFKGRFAFSLETSASTAYLQINNLKNEDTATYFC |
| | | ARDWGPYWGQGTLVIVSASPNSASHSGSAPQTSSAPGSQNIMMTQSPSS |
| | | LAVSAGEKVTVNCKSSQSVLYSSNQMNYLAWYQQKPGQSPKLLIYWAS |
| | | TRESGVPDRFTGSGSGTDFTLTISSVQTEDLAVYYCLQYLSSWTFGGGTK |
| | | LEIKGGSSRSSSSGGGGSGGGGAGQPLQERAQAWGERLRARMEEMGSR |
| | | TRDRLDEVKEQVAEVRAKLEEQAQQIRLQAEAAQARLKSRFEPLAEDM |
| | | QRQWAGQVEKVQAAEGTSAAPVPSDNHGGSSRSSSSGGGGSGGGGEVQ |
| | | LLESGGGLVQPGGSLRLSCAASGFTFSSFPMAWVRQAPGKGLEWVSTIST |
| | | SGGRTYYRDSVKGRFTISRDNSKNTLYLQMNSLRAEDTAVYYCAKFRQ |
| | | YSGGFDYWGQGTLVTVSSSPNSASHSGSAPQTSSAPGSQDIQLTQPNSVS |
| | | TSLGSTVKLSCTLSSGNIENNYVHWYQLYEGRSPTTMIYDDDKRPDGVP |
| | | DRFSGSIDRSSNSAFLTIHNVAIEDEAIYFCHSYVSSFNVFGGGTKLTVLR |
| | | GGGGSHHHHHHHH |
| CM04D | 9 | KVEQAVETEPEPELRQQTEWQSGQRWELALGRFWDYLRWVQTLSEQV |
| | | QEELLSSQVTQELRALMDETMKELKAYKSELEEQLTPVAEETRARLSKE |
| | | LQAAQARLGADMEDVCGRLVQYRGEVQAMLGQSTEELRVRLASALRA |
| | | LRKRLLRDADDLQKRLAVYQAGAREGAERGLSAIRERLGPLVEQGRVR |
| | | AATVGSLAGQPLQERAQAWGERLRARMEEMGSRTRDRLDEVKEQVAE |
| | | VRAKLEEQAQQIRLQAEAFQARLKSWFEPLVEDMQRQWAGLVEKVQA |
| | | AVGTSAAPVPSDNHGGSSRSSSSGGGGSGGGGEVQLLESGGGLVQPGGS |
| | | LRLSCAASGFTFSSFPMAWVRQAPGKGLEWVSTISTSGGRTYYRDSVKG |
| | | RFTISRDNSKNTLYLQMNSLRAEDTAVYYCAKFRQYSGGFDYWGQGTL |
| | | VTVSSSPNSASHSGSAPQTSSAPGSQDIQLTQPNSVSTSLGSTVKLSCTLSS |
| | | GNIENNYVHWYQLYEGRSPTTMIYDDDKRPDGVPDRFSGSIDRSSNSAFL |
| | | TIHNVAIEDEAIYFCHSYVSSFNVFGGGTKLTVLRGGGGSHHHHHHHH |
| CM04E | 10 | QIQLVQSGPELKKPGETVKISCKASGYTFKNYGMNWVKQAPGKGLKW |
| | | MGWINTYTGQPIYANDFKGRFAFSLETSASTAYLQINNLKNEDTATYFC |
| | | ARDWGPYWGQGTLVIVSASPNSASHSGSAPQTSSAPGSQNIMMTQSPSS |
| | | LAVSAGEKVTVNCKSSQSVLYSSNQMNYLAWYQQKPGQSPKLLIYWAS |
| | | TRESGVPDRFTGSGSGTDFTLTISSVQTEDLAVYYCLQYLSSWTFGGGTK |
| | | LEIKGGSSRSSSSGGGGSGGGGKVEQAVETEPEPELRQQTEWQSGQRWE |
| | | LALGRFWDYLRWVQTLSEQVQEELLSSQVTQELRALMDETMKELKAYK |
| | | SELEEQLTPVAEETRARLSKELQAAQARLGADMEDVCGRLVQYRGEVQ |
| | | AMLGQSTEELRVRLASALRALRKRLLRDADDLQKRLAVYQAGAREGAE |
| | | RGLSAIRERLGPLVEQGRVRAATVGSLAGQPLQERAQAWGERLRARME |
| | | EMGSRTRDRLDEVKEQVAEVRAKLEEQAQQIRLQAEAFQARLKSWFEP |
| | | LVEDMQRQWAGLVEKVQAAVGTSAAPVPSDNHGGSSRSSSSGGGGSGG |
| | | GGEVOLLESGGGLVQPGGSLRLSCAASGFTFSSFPMAWVRQAPGKGLE |
| | | WVSTISTSGGRTYYRDSVKGRFTISRDNSKNTLYLQMNSLRAEDTAVYY |
| | | CAKFRQYSGGFDYWGQGTLVTVSSSPNSASHSGSAPQTSSAPGSQDIQLT |
| | | QPNSVSTSLGSTVKLSCTLSSGNIENNYVHWYQLYEGRSPTTMIYDDDK |
| | | RPDGVPDRFSGSIDRSSNSAFLTIHNVAIEDEAIYFCHSYVSSFNVFGGGT |
| | | KLTVLRGGGGSHHHHHHHH |
| CM04F | 11 | QIQLVQSGPELKKPGETVKISCKASGYTFKNYGMNWVKQAPGKGLKW |
| | | MGWINTYTGQPIYANDFKGRFAFSLETSASTAYLQINNLKNEDTATYFC |
| | | ARDWGPYWGQGTLVIVSASPNSASHSGSAPQTSSAPGSQNIMMTQSPSS |
| | | LAVSAGEKVTVNCKSSQSVLYSSNQMNYLAWYQQKPGQSPKLLIYWAS |
| | | TRESGVPDRFTGSGSGTDFTLTISSVQTEDLAVYYCLQYLSSWTFGGGTK |
| | | LEIKGGSSRSSSSGGGGSGGGGEVRLQQSGPDLIKPGASVKMSCKASGYT |
| | | FTGYVMHWVKQRPGQGLEWIGFINPYNDDIQSNERFRGKATLTSDKSST |
| | | TAYMELSSLTSEDSAVYYCARGAGYNFDGAYRFFDFWGQGTTLTVSSSP |
| | | NSASHSGSAPQTSSAPGSQDVVMTQSPLSLPVSLGDQASISCRSSQRLVHS |
| | | NGNTYLHWYLQKPGQSPKLLIYRVSNRFPGVPDRFSGSGSGTDFTLKISR |
| | | VEAEDLGIYFCSQSTHVPYTFGGGTKLEIKGGGGSHHHHHHHH |
| CM04G | 12 | AGQPLQERAQAWGERLRARMEEMGSRTRDRLDEVKEQVAEVRAKLEE |
| | | QAQQIRLQAEAAQARLKSRFEPLAEDMQRQWAGQVEKVQAAEGTSAAP |
| | | VPSDNHGGSSRSSSSGGGGSGGGGEVRLQQSGPDLIKPGASVKMSCKAS |
| | | GYTFTGYVMHWVKQRPGQGLEWIGFINPYNDDIQSNERFRGKATLTSDK |
| | | SSTTAYMELSSLTSEDSAVYYCARGAGYNFDGAYRFFDFWGQGTTLTVS |
| | | SSPNSASHSGSAPQTSSAPGSQDVVMTQSPLSLPVSLGDQASISCRSSQRL |
| | | VHSNGNTYLHWYLQKPGQSPKLLIYRVSNRFPGVPDRFSGSGSGTDFTL |
| | | KISRVEAEDLGIYFCSQSTHVPYTFGGGTKLEIKGGGGSHHHHHHHH |
| CM04H | 13 | QIQLVQSGPELKKPGETVKISCKASGYTFKNYGMNWVKQAPGKGLKW |
| | | MGWINTYTGQPIYANDFKGRFAFSLETSASTAYLQINNLKNEDTATYFC |
| | | ARDWGPYWGQGTLVIVSASPNSASHSGSAPQTSSAPGSQNIMMTQSPSS |
| | | LAVSAGEKVTVNCKSSQSVLYSSNQMNYLAWYQQKPGQSPKLLIYWAS |
| | | TRESGVPDRFTGSGSGTDFTLTISSVQTEDLAVYYCLQYLSSWTFGGGTK |
| | | LEIKGGSSRSSSSGGGGSGGGGAGQPLQERAQAWGERLRARMEEMGSR |
| | | TRDRLDEVKEQVAEVRAKLEEQAQQIRLQAEAAQARLKSRFEPLAEDM |
| | | QRQWAGQVEKVQAAEGTSAAPVPSDNHGGSSRSSSSGGGGSGGGGEVR |
| | | LQQSGPDLIKPGASVKMSCKASGYTFTGYVMHWVKQRPGQGLEWIGFI |
| | | NPYNDDIQSNERFRGKATLTSDKSSTTAYMELSSLTSEDSAVYYCARGA |
| | | GYNFDGAYRFFDFWGQGTTLTVSSSPNSASHSGSAPQTSSAPGSQDVVM |
| | | TQSPLSLPVSLGDQASISCRSSQRLVHSNGNTYLHWYLQKPGQSPKLLIY |
| | | RVSNRFPGVPDRFSGSGSGTDFTLKISRVEAEDLGIYFCSQSTHVPYTFGG |
| | | GTKLEIKGGGGSHHHHHHHH |
| CM04I | 14 | KVEQAVETEPEPELRQQTEWQSGQRWELALGRFWDYLRWVQTLSEQV |
| | | QEELLSSQVTQELRALMDETMKELKAYKSELEEQLTPVAEETRARLSKE |
| | | LQAAQARLGADMEDVCGRLVQYRGEVQAMLGQSTEELRVRLASALRA |
| | | LRKRLLRDADDLQKRLAVYQAGAREGAERGLSAIRERLGPLVEQGRVR |
| | | AATVGSLAGQPLQERAQAWGERLRARMEEMGSRTRDRLDEVKEQVAE |
| | | VRAKLEEQAQQIRLQAEAFQARLKSWFEPLVEDMQRQWAGLVEKVQA |
| | | AVGTSAAPVPSDNHGGSSRSSSSGGGGSGGGGEVRLQQSGPDLIKPGAS |
| | | VKMSCKASGYTFTGYVMHWVKQRPGQGLEWIGFINPYNDDIQSNERFR |
| | | GKATLTSDKSSTTAYMELSSLTSEDSAVYYCARGAGYNFDGAYRFFDF |
| | | WGQGTTLTVSSSPNSASHSGSAPQTSSAPGSQDVVMTQSPLSLPVSLGDQ |
| | | ASISCRSSQRLVHSNGNTYLHWYLQKPGQSPKLLIYRVSNRFPGVPDRFS |
| | | GSGSGTDFTLKISRVEAEDLGIYFCSQSTHVPYTFGGGTKLEIKGGGGSH |
| | | HHHHHHH |
| CM04J | 15 | QIQLVQSGPELKKPGETVKISCKASGYTFKNYGMNWVKQAPGKGLKW |
| | | MGWINTYTGQPIYANDFKGRFAFSLETSASTAYLQINNLKNEDTATYFC |
| | | ARDWGPYWGQGTLVIVSASPNSASHSGSAPQTSSAPGSQNIMMTQSPSS |
| | | LAVSAGEKVTVNCKSSQSVLYSSNQMNYLAWYQQKPGQSPKLLIYWAS |
| | | TRESGVPDRFTGSGSGTDFTLTISSVQTEDLAVYYCLQYLSSWTFGGGTK |
| | | LEIKGGSSRSSSSGGGGSGGGGKVEQAVETEPEPELRQQTEWQSGQRWE |
| | | LALGRFWDYLRWVQTLSEQVQEELLSSQVTQELRALMDETMKELKAYK |
| | | SELEEQLTPVAEETRARLSKELQAAQARLGADMEDVCGRLVQYRGEVQ |
| | | AMLGQSTEELRVRLASALRALRKRLLRDADDLQKRLAVYQAGAREGAE |
| | | RGLSAIRERLGPLVEQGRVRAATVGSLAGQPLQERAQAWGERLRARME |
| | | EMGSRTRDRLDEVKEQVAEVRAKLEEQAQQIRLQAEAFQARLKSWFEP |
| | | LVEDMQRQWAGLVEKVQAAVGTSAAPVPSDNHGGSSRSSSSGGGGSGG |
| | | GGEVRLQQSGPDLIKPGASVKMSCKASGYTFTGYVMHWVKQRPGQGLE |
| | | WIGFINPYNDDIQSNERFRGKATLTSDKSSTTAYMELSSLTSEDSAVYYC |
| | | ARGAGYNFDGAYRFFDFWGQGTTLTVSSSPNSASHSGSAPQTSSAPGSQ |
| | | DVVMTQSPLSLPVSLGDQASISCRSSQRLVHSNGNTYLHWYLQKPGQSP |
| | | KLLIYRVSNRFPGVPDRFSGSGSGTDFTLKISRVEAEDLGIYFCSQSTHVP |
| | | YTFGGGTKLEIKGGGGSHHHHHHHH |
| CM04K | 16 | QIQLVQSGPELKKPGETVKISCKASGYTFKNYGMNWVKQAPGKGLKW |
| | | MGWINTYTGQPIYANDFKGRFAFSLETSASTAYLQINNLKNEDTATYFC |
| | | ARDWGPYWGQGTLVIVSASPNSASHSGSAPQTSSAPGSQNIMMTQSPSS |
| | | LAVSAGEKVTVNCKSSQSVLYSSNQMNYLAWYQQKPGQSPKLLIYWAS |
| | | TRESGVPDRFTGSGSGTDFTLTISSVQTEDLAVYYCLQYLSSWTFGGGTK |
| | | LEIKGGSSRSSSSGGGGSGGGGQIQLVQSGPGLKKPGGSVRISCAASGYTF |
| | | TNYGMNWVKQAPGKGLRWMGWINTHTGEPTYADDFKGRFTFSLDTSK |
| | | STAYLQINSLRAEDTATYFCTRRGYDWYFDVWGQGTTVTVSSSPNSASH |
| | | SGSAPQTSSAPGSQDIQMTQSPSSMSASLGDRVTITCRASQDINSYLSWFQ |
| | | QKPGKSPKTLIYRANRLVDGVPSRFSGSGSGTDYTLTISSLQYEDFGIYYC |
| | | QQYDESPWTFGGGTKLEIKGGGGSHHHHHHHH |
| CM04L | 17 | AGQPLQERAQAWGERLRARMEEMGSRTRDRLDEVKEQVAEVRAKLEE |
| | | QAQQIRLQAEAAQARLKSRFEPLAEDMQRQWAGQVEKVQAAEGTSAAP |
| | | VPSDNHGGSSRSSSSGGGGSGGGGQIQLVQSGPGLKKPGGSVRISCAASG |
| | | YTFTNYGMNWVKQAPGKGLRWMGWINTHTGEPTYADDFKGRFTFSLD |
| | | TSKSTAYLQINSLRAEDTATYFCTRRGYDWYFDVWGQGTTVTVSSSPNS |
| | | ASHSGSAPQTSSAPGSQDIQMTQSPSSMSASLGDRVTITCRASQDINSYLS |
| | | WFQQKPGKSPKTLIYRANRLVDGVPSRFSGSGSGTDYTLTISSLQYEDFGI |
| | | YYCQQYDESPWTFGGGTKLEIKGGGGSHHHHHHHH |
| CM04M | 18 | QIQLVQSGPELKKPGETVKISCKASGYTFKNYGMNWVKQAPGKGLKW |
| | | MGWINTYTGQPIYANDFKGRFAFSLETSASTAYLQINNLKNEDTATYFC |
| | | ARDWGPYWGQGTLVIVSASPNSASHSGSAPQTSSAPGSQNIMMTQSPSS |
| | | LAVSAGEKVTVNCKSSQSVLYSSNQMNYLAWYQQKPGQSPKLLIYWAS |
| | | TRESGVPDRFTGSGSGTDFTLTISSVQTEDLAVYYCLQYLSSWTFGGGTK |
| | | LEIKGGSSRSSSSGGGGSGGGGAGQPLQERAQAWGERLRARMEEMGSR |
| | | TRDRLDEVKEQVAEVRAKLEEQAQQIRLQAEAAQARLKSRFEPLAEDM |
| | | QRQWAGQVEKVQAAEGTSAAPVPSDNH5GGSSRSSSSGGGGSGGGGQI |
| | | QLVQSGPGLKKPGGSVRISCAASGYTFTNYGMNWVKQAPGKGLRWMG |
| | | WINTHTGEPTYADDFKGRFTFSLDTSKSTAYLQINSLRAEDTATYFCTRR |
| | | GYDWYFDVWGQGTTVTVSSSPNSASHSGSAPQTSSAPGSQDIQMTQSPS |
| | | SMSASLGDRVTITCRASQDINSYLSWFQQKPGKSPKTLIYRANRLVDGVP |
| | | SRFSGSGSGTDYTLTISSLQYEDFGIYYCQQYDESPWTFGGGTKLEIKGG |
| | | GGSHHHHHHHH |
| CM04N | 19 | KVEQAVETEPEPELRQQTEWQSGQRWELALGRFWDYLRWVQTLSEQV |
| | | QEELLSSQVTQELRALMDETMKELKAYKSELEEQLTPVAEETRARLSKE |
| | | LQAAQARLGADMEDVCGRLVQYRGEVQAMLGQSTEELRVRLASALRA |
| | | LRKRLLRDADDLQKRLAVYQAGAREGAERGLSAIRERLGPLVEQGRVR |
| | | AATVGSLAGQPLQERAQAWGERLRARMEEMGSRTRDRLDEVKEQVAE |
| | | VRAKLEEQAQQIRLQAEAFQARLKSWFEPLVEDMQRQWAGLVEKVQA |
| | | AVGTSAAPVPSDNHGGSSRSSSSGGGGSGGGGQIQLVQSGPGLKKPGGS |
| | | VRISCAASGYTFTNYGMNWVKQAPGKGLRWMGWINTHTGEPTYADDF |
| | | KGRFTFSLDTSKSTAYLQINSLRAEDTATYFCTRRGYDWYFDVWGQGTT |
| | | VTVSSSPNSASHSGSAPQTSSAPGSQDIQMTQSPSSMSASLGDRVTITCRA |
| | | SQDINSYLSWFQQKPGKSPKTLIYRANRLVDGVPSRFSGSGSGTDYTLTIS |
| | | SLQYEDFGIYYCQQYDESPWTFGGGTKLEIKGGGGSHHHHHHHH |
| CM04O | 20 | QIQLVQSGPELKKPGETVKISCKASGYTFKNYGMNWVKQAPGKGLKW |
| | | MGWINTYTGQPIYANDFKGRFAFSLETSASTAYLQINNLKNEDTATYFC |
| | | ARDWGPYWGQGTLVIVSASPNSASHSGSAPQTSSAPGSQNIMMTQSPSS |
| | | LAVSAGEKVTVNCKSSQSVLYSSNQMNYLAWYQQKPGQSPKLLIYWAS |
| | | TRESGVPDRFTGSGSGTDFTLTISSVQTEDLAVYYCLQYLSSWTFGGGTK |
| | | LEIKGGSSRSSSSGGGGSGGGGKVEQAVETEPEPELRQQTEWQSGQRWE |
| | | LALGRFWDYLRWVQTLSEQVQEELLSSQVTQELRALMDETMKELKAYK |
| | | SELEEQLTPVAEETRARLSKELQAAQARLGADMEDVCGRLVQYRGEVQ |
| | | AMLGQSTEELRVRLASALRALRKRLLRDADDLQKRLAVYQAGAREGAE |
| | | RGLSAIRERLGPLVEQGRVRAATVGSLAGQPLQERAQAWGERLRARME |
| | | EMGSRTRDRLDEVKEQVAEVRAKLEEQAQQIRLQAEAFQARLKSWFEP |
| | | LVEDMQRQWAGLVEKVQAAVGTSAAPVPSDNHGGSSRSSSSGGGGSGG |
| | | GGQIQLVQSGPGLKKPGGSVRISCAASGYTFTNYGMNWVKQAPGKGLR |
| | | WMGWINTHTGEPTYADDFKGRFTFSLDTSKSTAYLQINSLRAEDTATYF |
| | | CTRRGYDWYFDVWGQGTTVTVSSSPNSASHSGSAPQTSSAPGSQDIQMT |
| | | QSPSSMSASLGDRVTITCRASQDINSYLSWFQQKPGKSPKTLIYRANRLV |
| | | DGVPSRFSGSGSGTDYTLTISSLQYEDFGIYYCQQYDESPWTFGGGTKLEI |
| | | KGGGGSHHHHHHHH |
| CM04P | 21 | QIQLVQSGPELKKPGETVKISCKASGYTFKNYGMNWVKQAPGKGLKW |
| | | MGWINTYTGQPIYANDFKGRFAFSLETSASTAYLQINNLKNEDTATYFC |
| | | ARDWGPYWGQGTLVIVSASPNSASHSGSAPQTSSAPGSQNIMMTQSPSS |
| | | LAVSAGEKVTVNCKSSQSVLYSSNQMNYLAWYQQKPGQSPKLLIYWAS |
| | | TRESGVPDRFTGSGSGTDFTLTISSVQTEDLAVYYCLQYLSSWTFGGGTK |
| | | LEIKGGSSRSSSSGGGGSGGGGQVKLQQSGPELKKPGETVKISCKASGYT |
| | | FTNYGMNWVKQAPGKGLKWMGWINTYTGESTYADDFKGRFAFSLETS |
| | | ASAAYLQINNLKNEDTATYFCARFAIKGDYWGQGTTVTVSSSPNSASHS |
| | | GSAPQTSSAPGSQDIVLTQSPFSNPVTLGTSASISCRSTKSLLHSNGITYLY |
| | | WYLQKPGQSPQLLIYQMSNLASGVPDRESSSGSGTDFTLRISRVEAEDVG |
| | | VYYCAQNLEIPRTFGGGTKLEIKGGGGSHHHHHHHH |

TABLE 4
DNA Sequences of Multi-Specific Reagent Constructs

| | SEQ ID | |
|---|---|---|
| Construct: | NO: | Sequence: |
| CM04A | 22 | CAGATCCAGCTGGTGCAGTCCGGCCCAGAGCTGAAAAAGCCAGGCGA |
| | | GACCGTGAAGATCTCCTGCAAGGCCAGCGGCTACACTTTCAAGAATT |
| | | ATGGCATGAACTGGGTGAAGCAGGCCCCCGGCAAGGGGCTGAAGTG |
| | | GATGGGGTGGATTAACACCTATACCGGCCAGCCCATTTACGCCAACG |
| | | ATTTCAAGGGGAGATTCGCCTTTAGCCTGGAAACCAGCGCGAGCACC |
| | | GCCTATCTGCAGATCAACAATCTTAAGAATGAGGATACAGCCACCTA |
| | | CTTTTGCGCCCGGGATTGGGGCCCATACTGGGGCCAGGGCACCCTGG |
| | | TGATCGTGTCCGCTTCCCCCAATTCTGCCTCCCACAGCGGCAGCGCCC |
| | | CACAGACTAGCTCCGCCCCCGGTTCCCAGAATATCATGATGACACAG |
| | | AGCCCTTCCAGCCTGGCCGTGTCTGCCGGCGAAAAGGTGACCGTGAA |
| | | CTGCAAGTCTAGCCAGTCTGTGCTGTACAGCTCCAATCAGATGAACTA |
| | | TCTGGCCTGGTACCAGCAGAAGCCTGGCCAGAGCCCTAAGCTGCTGA |
| | | TTTACTGGGCCAGCACACGCGAGTCTGGGGTCCCTGACAGATTTACCG |
| | | GTAGCGGAAGCGGCACAGATTTCACACTGACAATCTCTTCTGTGCAG |
| | | ACCGAAGACCTCGCAGTGTACTACTGTCTGCAGTATCTGAGCTCTTGG |
| | | ACTTTCGGCGGGGGGACCAAGCTGGAAATTAAGGGTGGAAGCAGTAG |
| | | ATCATCCAGTTCTGGTGGGGGAGGATCCGGGGGCGGGGGTGAGGTTC |
| | | AGCTCCTTGAATCAGGCGGAGGGCTCGTCCAACCAGGCGGATCTTTG |
| | | CGGCTGTCATGTGCCGCATCCGGATTCACGTTCTCCTCCTTTCCGATG |
| | | GCATGGGTGAGGCAAGCTCCTGGAAAAGGTCTCGAGTGGGTATCCAC |
| | | CATAAGTACTTCTGGCGGGCGCACTTACTACAGGGATAGTGTGAAGG |
| | | GAAGGTTTACCATTTCCAGAGATAACTCAAAGAATACCCTGTACCTGC |
| | | AAATGAACTCCCTCAGGGCTGAAGATACGGCTGTGTACTACTGTGCT |
| | | AAATTTCGACAATACTCAGGTGGGTTTGACTATTGGGGCCAGGGGAC |
| | | ACTGGTGACTGTGTCATCAAGTCCCAATTCCGCCTCACATAGCGGAAG |
| | | TGCCCCACAAACTTCCTCCGCGCCAGGTTCTCAAGACATTCAATTGAC |
| | | GCAGCCGAATAGCGTCTCTACGAGCTTGGGGAGTACCGTTAAGCTTTC |
| | | ATGTACTCTTTCCTCCGGTAACATAGAGAACAACTACGTGCATTGGTA |
| | | CCAACTCTACGAGGGCAGGTCTCCAACGACTATGATTTACGATGACG |
| | | ATAAACGACCTGATGGGGTCCCTGACCGCTTTTCAGGAAGCATTGAC |
| | | AGAAGCTCTAATTCCGCGTTTTTGACTATCCACAACGTAGCGATTGAA |
| | | GATGAAGCAATTTACTTCTGTCATTCCTATGTATCCAGCTTTAACGTCT |
| | | TCGGAGGTGGAACTAAGTTGACGGTACTCCGAGGGGGAGGTGGGTCT |
| | | CACCACCATCACCACCACCATCAT |
| CM04B | 23 | GCTGGACAGCCGCTGCAAGAGCGGGCACAAGCCTGGGGGGAAAGAC |
| | | TCAGGGCCAGAATGGAAGAAATGGGATCAAGAACAAGGGACAGGCT |
| | | CGATGAGGTGAAGGAGCAAGTAGCAGAGGTTCGAGCAAAGCTGGAA |
| | | GAACAAGCTCAGCAGATTAGATTGCAAGCAGAGGCGGCTCAAGCACG |
| | | ACTCAAGAGCAGGTTCGAGCCATTGGCTGAAGATATGCAGCGACAAT |
| | | GGGCTGGTCAGGTTGAGAAAGTCCAAGCGGCGGAAGGGACCAGTGC |
| | | AGCACCGGTACCATCCGACAATCATGGTGGAAGCAGTAGATCATCCA |
| | | GTTCTGGTGGGGGAGGATCCGGGGGCGGGGGTGAGGTTCAGCTCCTT |
| | | GAATCAGGCGGAGGGCTCGTCCAACCAGGCGGATCTTTGCGGCTGTC |
| | | ATGTGCCGCATCCGGATTCACGTTCTCCTCCTTTCCGATGGCATGGGT |
| | | GAGGCAAGCTCCTGGAAAAGGTCTCGAGTGGGTATCCACCATAAGTA |
| | | CTTCTGGCGGGCGCACTTACTACAGGGATAGTGTGAAGGGAAGGTTT |
| | | ACCATTTCCAGAGATAACTCAAAGAATACCCTGTACCTGCAAATGAA |
| | | CTCCCTCAGGGCTGAAGATACGGCTGTGTACTACTGTGCTAAATTTCG |
| | | ACAATACTCAGGTGGGTTTGACTATTGGGGCCAGGGGACACTGGTGA |
| | | CTGTGTCATCAAGTCCCAATTCCGCCTCACATAGCGGAAGTGCCCCAC |
| | | AAACTTCCTCCGCGCCAGGTTCTCAAGACATTCAATTGACGCAGCCGA |
| | | ATAGCGTCTCTACGAGCTTGGGGAGTACCGTTAAGCTTTCATGTACTC |
| | | TTTCCTCCGGTAACATAGAGAACAACTACGTGCATTGGTACCAACTCT |
| | | ACGAGGGCAGGTCTCCAACGACTATGATTTACGATGACGATAAACGA |
| | | CCTGATGGGGTCCCTGACCGCTTTTCAGGAAGCATTGACAGAAGCTCT |
| | | AATTCCGCGTTTTTGACTATCCACAACGTAGCGATTGAAGATGAAGCA |
| | | ATTTACTTCTGTCATTCCTATGTATCCAGCTTTAACGTCTTCGGAGGTG |
| | | GAACTAAGTTGACGGTACTCCGAGGGGGAGGTGGGTCTCACCACCAT |
| | | CACCACCACCATCAT |
| CM04C | 24 | CAGATCCAGCTGGTGCAGTCCGGCCCAGAGCTGAAAAAGCCAGGCGA |
| | | GACCGTGAAGATCTCCTGCAAGGCCAGCGGCTACACTTTCAAGAATT |
| | | ATGGCATGAACTGGGTGAAGCAGGCCCCCGGCAAGGGGCTGAAGTG |
| | | GATGGGGTGGATTAACACCTATACCGGCCAGCCCATTTACGCCAACG |
| | | ATTTCAAGGGGAGATTCGCCTTTAGCCTGGAAACCAGCGCGAGCACC |
| | | GCCTATCTGCAGATCAACAATCTTAAGAATGAGGATACAGCCACCTA |
| | | CTTTTGCGCCCGGGATTGGGGCCCATACTGGGGCCAGGGCACCCTGG |
| | | TGATCGTGTCCGCTTCCCCCAATTCTGCCTCCCACAGCGGCAGCGCCC |
| | | CACAGACTAGCTCCGCCCCCGGTTCCCAGAATATCATGATGACACAG |
| | | AGCCCTTCCAGCCTGGCCGTGTCTGCCGGCGAAAAGGTGACCGTGAA |
| | | CTGCAAGTCTAGCCAGTCTGTGCTGTACAGCTCCAATCAGATGAACTA |
| | | TCTGGCCTGGTACCAGCAGAAGCCTGGCCAGAGCCCTAAGCTGCTGA |
| | | TTTACTGGGCCAGCACACGCGAGTCTGGGGTCCCTGACAGATTTACCG |
| | | GTAGCGGAAGCGGCACAGATTTCACACTGACAATCTCTTCTGTGCAG |
| | | ACCGAAGACCTCGCAGTGTACTACTGTCTGCAGTATCTGAGCTCTTGG |
| | | ACTTTCGGCGGGGGGACCAAGCTGGAAATTAAGGGCGGATCTAGTCG |
| | | GTCATCCTCCAGTGGTGGTGGGGGTAGCGGGGGAGGCGGTGCTGGAC |
| | | AGCCGCTGCAAGAGCGGGCACAAGCCTGGGGGGAAAGACTCAGGGC |
| | | CAGAATGGAAGAAATGGGATCAAGAACAAGGGACAGGCTCGATGAG |
| | | GTGAAGGAGCAAGTAGCAGAGGTTCGAGCAAAGCTGGAAGAACAAG |
| | | CTCAGCAGATTAGATTGCAAGCAGAGGCGGCTCAAGCACGACTCAAG |
| | | AGCAGGTTCGAGCCATTGGCTGAAGATATGCAGCGACAATGGGCTGG |
| | | TCAGGTTGAGAAAGTCCAAGCGGCGGAAGGGACCAGTGCAGCACCG |
| | | GTACCATCCGACAATCATGGTGGAAGCAGTAGATCATCCAGTTCTGG |
| | | TGGGGGAGGATCCGGGGGCGGGGGTGAGGTTCAGCTCCTTGAATCAG |
| | | GCGGAGGGCTCGTCCAACCAGGCGGATCTTTGCGGCTGTCATGTGCC |
| | | GCATCCGGATTCACGTTCTCCTCCTTTCCGATGGCATGGGTGAGGCAA |
| | | GCTCCTGGAAAAGGTCTCGAGTGGGTATCCACCATAAGTACTTCTGGC |
| | | GGGCGCACTTACTACAGGGATAGTGTGAAGGGAAGGTTTACCATTTC |
| | | CAGAGATAACTCAAAGAATACCCTGTACCTGCAAATGAACTCCCTCA |
| | | GGGCTGAAGATACGGCTGTGTACTACTGTGCTAAATTTCGACAATACT |
| | | CAGGTGGGTTTGACTATTGGGGCCAGGGGACACTGGTGACTGTGTCA |
| | | TCAAGTCCCAATTCCGCCTCACATAGCGGAAGTGCCCCACAAACTTCC |
| | | TCCGCGCCAGGTTCTCAAGACATTCAATTGACGCAGCCGAATAGCGT |
| | | CTCTACGAGCTTGGGGAGTACCGTTAAGCTTTCATGTACTCTTTCCTC |
| | | CGGTAACATAGAGAACAACTACGTGCATTGGTACCAACTCTACGAGG |
| | | GCAGGTCTCCAACGACTATGATTTACGATGACGATAAACGACCTGAT |
| | | GGGGTCCCTGACCGCTTTTCAGGAAGCATTGACAGAAGCTCTAATTCC |
| | | GCGTTTTTGACTATCCACAACGTAGCGATTGAAGATGAAGCAATTTAC |
| | | TTCTGTCATTCCTATGTATCCAGCTTTAACGTCTTCGGAGGTGGAACT |
| | | AAGTTGACGGTACTCCGAGGGGGAGGTGGGTCTCACCACCATCACCA |
| | | CCACCATCAT |
| CM04D | 25 | AAGGTGGAGCAAGCGGTGGAGACAGAGCCGGAGCCCGAGCTGCGCC |
| | | AGCAGACCGAGTGGCAGAGCGGCCAGCGCTGGGAACTGGCACTGGG |
| | | TCGCTTTTGGGATTACCTGCGCTGGGTGCAGACACTGTCTGAGCAGGT |
| | | GCAGGAGGAGCTGCTCAGCTCCCAGGTCACCCAGGAACTGAGGGCGC |
| | | TGATGGACGAGACCATGAAGGAGTTGAAGGCCTACAAATCGGAACTG |
| | | GAGGAACAACTGACCCCGGTGGCGGAGGAGACGCGGGCACGGCTGT |
| | | CCAAGGAGCTGCAGGCGGCGCAGGCCCGGCTGGGCGCGGACATGGA |
| | | GGACGTGTGCGGCCGCCTGGTGCAGTACCGCGGCGAGGTGCAGGCCA |
| | | TGCTCGGCCAGAGCACCGAGGAGCTGCGGGTGCGCCTCGCCTCCGCC |
| | | CTGCGCGCCCTGCGTAAGCGGCTCCTCCGCGATGCCGATGACCTGCA |
| | | GAAGCGCCTGGCAGTGTACCAGGCCGGGGCCCGCGAGGGCGCCGAG |
| | | CGCGGCCTCAGCGCCATCCGCGAGCGCCTGGGGCCCCTGGTGGAACA |
| | | GGGCCGCGTGCGGGCCGCCACTGTGGGCTCCCTGGCCGGCCAGCCGC |
| | | TACAGGAGCGGGCCCAGGCCTGGGGCGAGCGGCTGCGCGCGCGGAT |
| | | GGAGGAGATGGGCAGCCGGACCCGCGACCGCCTGGACGAGGTGAAG |
| | | GAGCAGGTGGCGGAGGTGCGCGCCAAGCTGGAGGAGCAGGCCCAGC |
| | | AGATACGCCTGCAGGCCGAGGCCTTCCAGGCCCGCCTCAAGAGCTGG |
| | | TTCGAGCCCCTGGTGGAAGACATGCAGCGCCAGTGGGCCGGGCTGGT |
| | | GGAGAAGGTGCAGGCTGCCGTGGGCACCAGCGCCGCCCCTGTGCCCA |
| | | GCGACAATCACGGTGGAAGCAGTAGATCATCCAGTTCTGGTGGGGGA |
| | | GGATCCGGGGGCGGGGGTGAGGTTCAGCTCCTTGAATCAGGCGGAGG |
| | | GCTCGTCCAACCAGGCGGATCTTTGCGGCTGTCATGTGCCGCATCCGG |
| | | ATTCACGTTCTCCTCCTTTCCGATGGCATGGGTGAGGCAAGCTCCTGG |
| | | AAAAGGTCTCGAGTGGGTATCCACCATAAGTACTTCTGGCGGGCGCA |
| | | CTTACTACAGGGATAGTGTGAAGGGAAGGTTTACCATTTCCAGAGAT |
| | | AACTCAAAGAATACCCTGTACCTGCAAATGAACTCCCTCAGGGCTGA |
| | | AGATACGGCTGTGTACTACTGTGCTAAATTTCGACAATACTCAGGTGG |
| | | GTTTGACTATTGGGGCCAGGGGACACTGGTGACTGTGTCATCAAGTCC |
| | | CAATTCCGCCTCACATAGCGGAAGTGCCCCACAAACTTCCTCCGCGCC |
| | | AGGTTCTCAAGACATTCAATTGACGCAGCCGAATAGCGTCTCTACGA |
| | | GCTTGGGGAGTACCGTTAAGCTTTCATGTACTCTTTCCTCCGGTAACA |
| | | TAGAGAACAACTACGTGCATTGGTACCAACTCTACGAGGGCAGGTCT |
| | | CCAACGACTATGATTTACGATGACGATAAACGACCTGATGGGGTCCC |
| | | TGACCGCTTTTCAGGAAGCATTGACAGAAGCTCTAATTCCGCGTTTTT |
| | | GACTATCCACAACGTAGCGATTGAAGATGAAGCAATTTACTTCTGTCA |
| | | TTCCTATGTATCCAGCTTTAACGTCTTCGGAGGTGGAACTAAGTTGAC |
| | | GGTACTCCGAGGGGGAGGTGGGTCTCACCACCATCACCACCACCATC |
| | | AT |
| CM04E | 26 | CAGATCCAGCTGGTGCAGTCCGGCCCAGAGCTGAAAAAGCCAGGCGA |
| | | GACCGTGAAGATCTCCTGCAAGGCCAGCGGCTACACTTTCAAGAATT |
| | | ATGGCATGAACTGGGTGAAGCAGGCCCCCGGCAAGGGGCTGAAGTG |
| | | GATGGGGTGGATTAACACCTATACCGGCCAGCCCATTTACGCCAACG |
| | | ATTTCAAGGGGAGATTCGCCTTTAGCCTGGAAACCAGCGCGAGCACC |
| | | GCCTATCTGCAGATCAACAATCTTAAGAATGAGGATACAGCCACCTA |
| | | CTTTTGCGCCCGGGATTGGGGCCCATACTGGGGCCAGGGCACCCTGG |
| | | TGATCGTGTCCGCTTCCCCCAATTCTGCCTCCCACAGCGGCAGCGCCC |
| | | CACAGACTAGCTCCGCCCCCGGTTCCCAGAATATCATGATGACACAG |
| | | AGCCCTTCCAGCCTGGCCGTGTCTGCCGGCGAAAAGGTGACCGTGAA |
| | | CTGCAAGTCTAGCCAGTCTGTGCTGTACAGCTCCAATCAGATGAACTA |
| | | TCTGGCCTGGTACCAGCAGAAGCCTGGCCAGAGCCCTAAGCTGCTGA |
| | | TTTACTGGGCCAGCACACGCGAGTCTGGGGTCCCTGACAGATTTACCG |
| | | GTAGCGGAAGCGGCACAGATTTCACACTGACAATCTCTTCTGTGCAG |
| | | ACCGAAGACCTCGCAGTGTACTACTGTCTGCAGTATCTGAGCTCTTGG |
| | | ACTTTCGGCGGGGGGACCAAGCTGGAAATTAAGGGCGGATCTAGTCG |
| | | GTCATCCTCCAGTGGTGGTGGGGGTAGCGGGGGAGGCGGTAAGGTGG |
| | | AGCAAGCGGTGGAGACAGAGCCGGAGCCCGAGCTGCGCCAGCAGAC |
| | | CGAGTGGCAGAGCGGCCAGCGCTGGGAACTGGCACTGGGTCGCTTTT |
| | | GGGATTACCTGCGCTGGGTGCAGACACTGTCTGAGCAGGTGCAGGAG |
| | | GAGCTGCTCAGCTCCCAGGTCACCCAGGAACTGAGGGCGCTGATGGA |
| | | CGAGACCATGAAGGAGTTGAAGGCCTACAAATCGGAACTGGAGGAA |
| | | CAACTGACCCCGGTGGCGGAGGAGACGCGGGCACGGCTGTCCAAGG |
| | | AGCTGCAGGCGGCGCAGGCCCGGCTGGGCGCGGACATGGAGGACGT |
| | | GTGCGGCCGCCTGGTGCAGTACCGCGGCGAGGTGCAGGCCATGCTCG |
| | | GCCAGAGCACCGAGGAGCTGCGGGTGCGCCTCGCCTCCGCCCTGCGC |
| | | GCCCTGCGTAAGCGGCTCCTCCGCGATGCCGATGACCTGCAGAAGCG |
| | | CCTGGCAGTGTACCAGGCCGGGGCCCGCGAGGGCGCCGAGCGCGGCC |
| | | TCAGCGCCATCCGCGAGCGCCTGGGGCCCCTGGTGGAACAGGGCCGC |
| | | GTGCGGGCCGCCACTGTGGGCTCCCTGGCCGGCCAGCCGCTACAGGA |
| | | GCGGGCCCAGGCCTGGGGCGAGCGGCTGCGCGCGCGGATGGAGGAG |
| | | ATGGGCAGCCGGACCCGCGACCGCCTGGACGAGGTGAAGGAGCAGG |
| | | TGGCGGAGGTGCGCGCCAAGCTGGAGGAGCAGGCCCAGCAGATACG |
| | | CCTGCAGGCCGAGGCCTTCCAGGCCCGCCTCAAGAGCTGGTTCGAGC |
| | | CCCTGGTGGAAGACATGCAGCGCCAGTGGGCCGGGCTGGTGGAGAAG |
| | | GTGCAGGCTGCCGTGGGCACCAGCGCCGCCCCTGTGCCCAGCGACAA |
| | | TCACGGTGGAAGCAGTAGATCATCCAGTTCTGGTGGGGGAGGATCCG |
| | | GGGGCGGGGGTGAGGTTCAGCTCCTTGAATCAGGCGGAGGGCTCGTC |
| | | CAACCAGGCGGATCTTTGCGGCTGTCATGTGCCGCATCCGGATTCACG |
| | | TTCTCCTCCTTTCCGATGGCATGGGTGAGGCAAGCTCCTGGAAAAGGT |
| | | CTCGAGTGGGTATCCACCATAAGTACTTCTGGCGGGCGCACTTACTAC |
| | | AGGGATAGTGTGAAGGGAAGGTTTACCATTTCCAGAGATAACTCAAA |
| | | GAATACCCTGTACCTGCAAATGAACTCCCTCAGGGCTGAAGATACGG |
| | | CTGTGTACTACTGTGCTAAATTTCGACAATACTCAGGTGGGTTTGACT |
| | | ATTGGGGCCAGGGGACACTGGTGACTGTGTCATCAAGTCCCAATTCC |
| | | GCCTCACATAGCGGAAGTGCCCCACAAACTTCCTCCGCGCCAGGTTCT |
| | | CAAGACATTCAATTGACGCAGCCGAATAGCGTCTCTACGAGCTTGGG |
| | | GAGTACCGTTAAGCTTTCATGTACTCTTTCCTCCGGTAACATAGAGAA |
| | | CAACTACGTGCATTGGTACCAACTCTACGAGGGCAGGTCTCCAACGA |
| | | CTATGATTTACGATGACGATAAACGACCTGATGGGGTCCCTGACCGCT |
| | | TTTCAGGAAGCATTGACAGAAGCTCTAATTCCGCGTTTTTGACTATCC |
| | | ACAACGTAGCGATTGAAGATGAAGCAATTTACTTCTGTCATTCCTATG |
| | | TATCCAGCTTTAACGTCTTCGGAGGTGGAACTAAGTTGACGGTACTCC |
| | | GAGGGGGAGGTGGGTCTCACCACCATCACCACCACCATCAT |
| CM04F | 27 | CAGATCCAGCTGGTGCAGTCCGGCCCAGAGCTGAAAAAGCCAGGCGA |
| | | GACCGTGAAGATCTCCTGCAAGGCCAGCGGCTACACTTTCAAGAATT |
| | | ATGGCATGAACTGGGTGAAGCAGGCCCCCGGCAAGGGGCTGAAGTG |
| | | GATGGGGTGGATTAACACCTATACCGGCCAGCCCATTTACGCCAACG |
| | | ATTTCAAGGGGAGATTCGCCTTTAGCCTGGAAACCAGCGCGAGCACC |
| | | GCCTATCTGCAGATCAACAATCTTAAGAATGAGGATACAGCCACCTA |
| | | CTTTTGCGCCCGGGATTGGGGCCCATACTGGGGCCAGGGCACCCTGG |
| | | TGATCGTGTCCGCTTCCCCCAATTCTGCCTCCCACAGCGGCAGCGCCC |
| | | CACAGACTAGCTCCGCCCCCGGTTCCCAGAATATCATGATGACACAG |
| | | AGCCCTTCCAGCCTGGCCGTGTCTGCCGGCGAAAAGGTGACCGTGAA |
| | | CTGCAAGTCTAGCCAGTCTGTGCTGTACAGCTCCAATCAGATGAACTA |
| | | TCTGGCCTGGTACCAGCAGAAGCCTGGCCAGAGCCCTAAGCTGCTGA |
| | | TTTACTGGGCCAGCACACGCGAGTCTGGGGTCCCTGACAGATTTACCG |
| | | GTAGCGGAAGCGGCACAGATTTCACACTGACAATCTCTTCTGTGCAG |
| | | ACCGAAGACCTCGCAGTGTACTACTGTCTGCAGTATCTGAGCTCTTGG |
| | | ACTTTCGGCGGGGGGACCAAGCTGGAAATTAAGGGTGGAAGCAGTAG |
| | | ATCATCCAGTAGTGGGGGGGGCGGATCTGGAGGTGGGGGAGAGGTG |
| | | CGGCTGCAGCAGTCCGGCCCCGACCTGATCAAGCCCGGCGCCTCCGT |
| | | GAAGATGTCTTGCAAAGCCTCCGGCTACACCTTCACAGGATATGTGAT |
| | | GCACTGGGTGAAGCAGAGGCCCGGCCAGGGACTGGAATGGATCGGCT |
| | | TCATCAACCCATACAACGATGACATTCAGTCCAACGAGAGATTTAGG |
| | | GGAAAGGCCACCCTGACCAGTGACAAATCAAGCACTACAGCCTACAT |
| | | GGAGCTGAGCTCACTGACCTCTGAGGACTCCGCCGTGTATTACTGTGC |
| | | CAGAGGCGCCGGCTACAATTTTGACGGCGCCTACCGGTTCTTCGACTT |
| | | CTGGGGGCAGGGCACCACCCTGACCGTGTCCAGCTCCCCCAATAGTG |
| | | CCTCCCATTCTGGCTCCGCCCCCCAGACTAGCTCTGCCCCTGGGTCCC |
| | | AGGACGTGGTGATGACCCAGTCCCCCCTGTCTCTGCCTGTGTCCCTGG |
| | | GAGACCAGGCCTCTATCAGCTGCCGGAGCTCTCAGAGGCTGGTGCAC |
| | | AGCAATGGGAATACATACCTGCACTGGTACCTGCAGAAGCCAGGCCA |
| | | GAGCCCCAAGCTGCTGATCTACAGAGTGAGCAACAGGTTCCCCGGAG |
| | | TCCCTGATAGGTTTAGCGGATCTGGCAGCGGCACCGACTTCACTCTGA |
| | | AGATTAGCAGAGTGGAGGCCGAGGATCTGGGCATCTACTTTTGCTCTC |
| | | AGTCCACCCATGTGCCTTACACCTTTGGGGGAGGCACCAAGCTGGAG |
| | | ATTAAGAGGGGGGGGGGGGCTCCCACCACCACCATCACCACCACCA |
| | | C |
| CM04G | 28 | GCTGGACAGCCGCTGCAAGAGCGGGCACAAGCCTGGGGGGAAAGAC |
| | | TCAGGGCCAGAATGGAAGAAATGGGATCAAGAACAAGGGACAGGCT |
| | | CGATGAGGTGAAGGAGCAAGTAGCAGAGGTTCGAGCAAAGCTGGAA |
| | | GAACAAGCTCAGCAGATTAGATTGCAAGCAGAGGCGGCTCAAGCACG |
| | | ACTCAAGAGCAGGTTCGAGCCATTGGCTGAAGATATGCAGCGACAAT |
| | | GGGCTGGTCAGGTTGAGAAAGTCCAAGCGGCGGAAGGGACCAGTGC |
| | | AGCACCGGTACCATCCGACAATCATGGTGGAAGCAGTAGATCATCCA |
| | | GTAGTGGGGGGGGCGGATCTGGAGGTGGGGGAGAGGTGCGGCTGCA |
| | | GCAGTCCGGCCCCGACCTGATCAAGCCCGGCGCCTCCGTGAAGATGT |
| | | CTTGCAAAGCCTCCGGCTACACCTTCACAGGATATGTGATGCACTGGG |
| | | TGAAGCAGAGGCCCGGCCAGGGACTGGAATGGATCGGCTTCATCAAC |
| | | CCATACAACGATGACATTCAGTCCAACGAGAGATTTAGGGGAAAGGC |
| | | CACCCTGACCAGTGACAAATCAAGCACTACAGCCTACATGGAGCTGA |
| | | GCTCACTGACCTCTGAGGACTCCGCCGTGTATTACTGTGCCAGAGGCG |
| | | CCGGCTACAATTTTGACGGCGCCTACCGGTTCTTCGACTTCTGGGGGC |
| | | AGGGCACCACCCTGACCGTGTCCAGCTCCCCCAATAGTGCCTCCCATT |
| | | CTGGCTCCGCCCCCCAGACTAGCTCTGCCCCTGGGTCCCAGGACGTGG |
| | | TGATGACCCAGTCCCCCCTGTCTCTGCCTGTGTCCCTGGGAGACCAGG |
| | | CCTCTATCAGCTGCCGGAGCTCTCAGAGGCTGGTGCACAGCAATGGG |
| | | AATACATACCTGCACTGGTACCTGCAGAAGCCAGGCCAGAGCCCCAA |
| | | GCTGCTGATCTACAGAGTGAGCAACAGGTTCCCCGGAGTCCCTGATA |
| | | GGTTTAGCGGATCTGGCAGCGGCACCGACTTCACTCTGAAGATTAGC |
| | | AGAGTGGAGGCCGAGGATCTGGGCATCTACTTTTGCTCTCAGTCCACC |
| | | CATGTGCCTTACACCTTTGGGGGAGGCACCAAGCTGGAGATTAAGAG |
| | | GGGCGGGGGCGGCTCCCACCACCACCATCACCACCACCAC |
| CM04H | 29 | CAGATCCAGCTGGTGCAGTCCGGCCCAGAGCTGAAAAAGCCAGGCGA |
| | | GACCGTGAAGATCTCCTGCAAGGCCAGCGGCTACACTTTCAAGAATT |
| | | ATGGCATGAACTGGGTGAAGCAGGCCCCCGGCAAGGGGCTGAAGTG |
| | | GATGGGGTGGATTAACACCTATACCGGCCAGCCCATTTACGCCAACG |
| | | ATTTCAAGGGGAGATTCGCCTTTAGCCTGGAAACCAGCGCGAGCACC |
| | | GCCTATCTGCAGATCAACAATCTTAAGAATGAGGATACAGCCACCTA |
| | | CTTTTGCGCCCGGGATTGGGGCCCATACTGGGGCCAGGGCACCCTGG |
| | | TGATCGTGTCCGCTTCCCCCAATTCTGCCTCCCACAGCGGCAGCGCCC |
| | | CACAGACTAGCTCCGCCCCCGGTTCCCAGAATATCATGATGACACAG |
| | | AGCCCTTCCAGCCTGGCCGTGTCTGCCGGCGAAAAGGTGACCGTGAA |
| | | CTGCAAGTCTAGCCAGTCTGTGCTGTACAGCTCCAATCAGATGAACTA |
| | | TCTGGCCTGGTACCAGCAGAAGCCTGGCCAGAGCCCTAAGCTGCTGA |
| | | TTTACTGGGCCAGCACACGCGAGTCTGGGGTCCCTGACAGATTTACCG |
| | | GTAGCGGAAGCGGCACAGATTTCACACTGACAATCTCTTCTGTGCAG |
| | | ACCGAAGACCTCGCAGTGTACTACTGTCTGCAGTATCTGAGCTCTTGG |
| | | ACTTTCGGCGGGGGGACCAAGCTGGAAATTAAGGGCGGATCTAGTCG |
| | | GTCATCCTCCAGTGGTGGTGGGGGTAGCGGGGGAGGCGGTGCTGGAC |
| | | AGCCGCTGCAAGAGCGGGCACAAGCCTGGGGGGAAAGACTCAGGGC |
| | | CAGAATGGAAGAAATGGGATCAAGAACAAGGGACAGGCTCGATGAG |
| | | GTGAAGGAGCAAGTAGCAGAGGTTCGAGCAAAGCTGGAAGAACAAG |
| | | CTCAGCAGATTAGATTGCAAGCAGAGGCGGCTCAAGCACGACTCAAG |
| | | AGCAGGTTCGAGCCATTGGCTGAAGATATGCAGCGACAATGGGCTGG |
| | | TCAGGTTGAGAAAGTCCAAGCGGCGGAAGGGACCAGTGCAGCACCG |
| | | GTACCATCCGACAATCATGGTGGAAGCAGTAGATCATCCAGTAGTGG |
| | | GGGGGGCGGATCTGGAGGTGGGGGAGAGGTGCGGCTGCAGCAGTCC |
| | | GGCCCCGACCTGATCAAGCCCGGCGCCTCCGTGAAGATGTCTTGCAA |
| | | AGCCTCCGGCTACACCTTCACAGGATATGTGATGCACTGGGTGAAGC |
| | | AGAGGCCCGGCCAGGGACTGGAATGGATCGGCTTCATCAACCCATAC |
| | | AACGATGACATTCAGTCCAACGAGAGATTTAGGGGAAAGGCCACCCT |
| | | GACCAGTGACAAATCAAGCACTACAGCCTACATGGAGCTGAGCTCAC |
| | | TGACCTCTGAGGACTCCGCCGTGTATTACTGTGCCAGAGGCGCCGGCT |
| | | ACAATTTTGACGGCGCCTACCGGTTCTTCGACTTCTGGGGGCAGGGCA |
| | | CCACCCTGACCGTGTCCAGCTCCCCCAATAGTGCCTCCCATTCTGGCT |
| | | CCGCCCCCCAGACTAGCTCTGCCCCTGGGTCCCAGGACGTGGTGATG |
| | | ACCCAGTCCCCCCTGTCTCTGCCTGTGTCCCTGGGAGACCAGGCCTCT |
| | | ATCAGCTGCCGGAGCTCTCAGAGGCTGGTGCACAGCAATGGGAATAC |
| | | ATACCTGCACTGGTACCTGCAGAAGCCAGGCCAGAGCCCCAAGCTGC |
| | | TGATCTACAGAGTGAGCAACAGGTTCCCCGGAGTCCCTGATAGGTTT |
| | | AGCGGATCTGGCAGCGGCACCGACTTCACTCTGAAGATTAGCAGAGT |
| | | GGAGGCCGAGGATCTGGGCATCTACTTTTGCTCTCAGTCCACCCATGT |
| | | GCCTTACACCTTTGGGGGAGGCACCAAGCTGGAGATTAAGAGGGGCG |
| | | GGGGCGGCTCCCACCACCACCATCACCACCACCAC |
| CM04I | 30 | AAGGTGGAGCAAGCGGTGGAGACAGAGCCGGAGCCCGAGCTGCGCC |
| | | AGCAGACCGAGTGGCAGAGCGGCCAGCGCTGGGAACTGGCACTGGG |
| | | TCGCTTTTGGGATTACCTGCGCTGGGTGCAGACACTGTCTGAGCAGGT |
| | | GCAGGAGGAGCTGCTCAGCTCCCAGGTCACCCAGGAACTGAGGGCGC |
| | | TGATGGACGAGACCATGAAGGAGTTGAAGGCCTACAAATCGGAACTG |
| | | GAGGAACAACTGACCCCGGTGGCGGAGGAGACGCGGGCACGGCTGT |
| | | CCAAGGAGCTGCAGGCGGCGCAGGCCCGGCTGGGCGCGGACATGGA |
| | | GGACGTGTGCGGCCGCCTGGTGCAGTACCGCGGCGAGGTGCAGGCCA |
| | | TGCTCGGCCAGAGCACCGAGGAGCTGCGGGTGCGCCTCGCCTCCGCC |
| | | CTGCGCGCCCTGCGTAAGCGGCTCCTCCGCGATGCCGATGACCTGCA |
| | | GAAGCGCCTGGCAGTGTACCAGGCCGGGGCCCGCGAGGGCGCCGAG |
| | | CGCGGCCTCAGCGCCATCCGCGAGCGCCTGGGGCCCCTGGTGGAACA |
| | | GGGCCGCGTGCGGGCCGCCACTGTGGGCTCCCTGGCCGGCCAGCCGC |
| | | TACAGGAGCGGGCCCAGGCCTGGGGCGAGCGGCTGCGCGCGCGGAT |
| | | GGAGGAGATGGGCAGCCGGACCCGCGACCGCCTGGACGAGGTGAAG |
| | | GAGCAGGTGGCGGAGGTGCGCGCCAAGCTGGAGGAGCAGGCCCAGC |
| | | AGATACGCCTGCAGGCCGAGGCCTTCCAGGCCCGCCTCAAGAGCTGG |
| | | TTCGAGCCCCTGGTGGAAGACATGCAGCGCCAGTGGGCCGGGCTGGT |
| | | GGAGAAGGTGCAGGCTGCCGTGGGCACCAGCGCCGCCCCTGTGCCCA |
| | | GCGACAATCACGGTGGAAGCAGTAGATCATCCAGTTCTGGTGGGGGA |
| | | GGATCCGGGGGCGGGGGTGAGGTGCGGCTGCAGCAGTCCGGCCCCGA |
| | | CCTGATCAAGCCCGGCGCCTCCGTGAAGATGTCTTGCAAAGCCTCCG |
| | | GCTACACCTTCACAGGATATGTGATGCACTGGGTGAAGCAGAGGCCC |
| | | GGCCAGGGACTGGAATGGATCGGCTTCATCAACCCATACAACGATGA |
| | | CATTCAGTCCAACGAGAGATTTAGGGGAAAGGCCACCCTGACCAGTG |
| | | ACAAATCAAGCACTACAGCCTACATGGAGCTGAGCTCACTGACCTCT |
| | | GAGGACTCCGCCGTGTATTACTGTGCCAGAGGCGCCGGCTACAATTTT |
| | | GACGGCGCCTACCGGTTCTTCGACTTCTGGGGGCAGGGCACCACCCT |
| | | GACCGTGTCCAGCTCCCCCAATAGTGCCTCCCATTCTGGCTCCGCCCC |
| | | CCAGACTAGCTCTGCCCCTGGGTCCCAGGACGTGGTGATGACCCAGT |
| | | CCCCCCTGTCTCTGCCTGTGTCCCTGGGAGACCAGGCCTCTATCAGCT |
| | | GCCGGAGCTCTCAGAGGCTGGTGCACAGCAATGGGAATACATACCTG |
| | | CACTGGTACCTGCAGAAGCCAGGCCAGAGCCCCAAGCTGCTGATCTA |
| | | CAGAGTGAGCAACAGGTTCCCCGGAGTCCCTGATAGGTTTAGCGGAT |
| | | CTGGCAGCGGCACCGACTTCACTCTGAAGATTAGCAGAGTGGAGGCC |
| | | GAGGATCTGGGCATCTACTTTTGCTCTCAGTCCACCCATGTGCCTTAC |
| | | ACCTTTGGGGGAGGCACCAAGCTGGAGATTAAGAGGGGCGGGGGCG |
| | | GCTCCCACCACCACCATCACCACCACCAC |
| CM04J | 31 | CAGATCCAGCTGGTGCAGTCCGGCCCAGAGCTGAAAAAGCCAGGCGA |
| | | GACCGTGAAGATCTCCTGCAAGGCCAGCGGCTACACTTTCAAGAATT |
| | | ATGGCATGAACTGGGTGAAGCAGGCCCCCGGCAAGGGGCTGAAGTG |
| | | GATGGGGTGGATTAACACCTATACCGGCCAGCCCATTTACGCCAACG |
| | | ATTTCAAGGGGAGATTCGCCTTTAGCCTGGAAACCAGCGCGAGCACC |
| | | GCCTATCTGCAGATCAACAATCTTAAGAATGAGGATACAGCCACCTA |
| | | CTTTTGCGCCCGGGATTGGGGCCCATACTGGGGCCAGGGCACCCTGG |
| | | TGATCGTGTCCGCTTCCCCCAATTCTGCCTCCCACAGCGGCAGCGCCC |
| | | CACAGACTAGCTCCGCCCCCGGTTCCCAGAATATCATGATGACACAG |
| | | AGCCCTTCCAGCCTGGCCGTGTCTGCCGGCGAAAAGGTGACCGTGAA |
| | | CTGCAAGTCTAGCCAGTCTGTGCTGTACAGCTCCAATCAGATGAACTA |
| | | TCTGGCCTGGTACCAGCAGAAGCCTGGCCAGAGCCCTAAGCTGCTGA |
| | | TTTACTGGGCCAGCACACGCGAGTCTGGGGTCCCTGACAGATTTACCG |
| | | GTAGCGGAAGCGGCACAGATTTCACACTGACAATCTCTTCTGTGCAG |
| | | ACCGAAGACCTCGCAGTGTACTACTGTCTGCAGTATCTGAGCTCTTGG |
| | | ACTTTCGGCGGGGGGACCAAGCTGGAAATTAAGGGCGGATCTAGTCG |
| | | GTCATCCTCCAGTGGTGGTGGGGGTAGCGGGGGAGGCGGTAAGGTGG |
| | | AGCAAGCGGTGGAGACAGAGCCGGAGCCCGAGCTGCGCCAGCAGAC |
| | | CGAGTGGCAGAGCGGCCAGCGCTGGGAACTGGCACTGGGTCGCTTTT |
| | | GGGATTACCTGCGCTGGGTGCAGACACTGTCTGAGCAGGTGCAGGAG |
| | | GAGCTGCTCAGCTCCCAGGTCACCCAGGAACTGAGGGCGCTGATGGA |
| | | CGAGACCATGAAGGAGTTGAAGGCCTACAAATCGGAACTGGAGGAA |
| | | CAACTGACCCCGGTGGCGGAGGAGACGCGGGCACGGCTGTCCAAGG |
| | | AGCTGCAGGCGGCGCAGGCCCGGCTGGGCGCGGACATGGAGGACGT |
| | | GTGCGGCCGCCTGGTGCAGTACCGCGGCGAGGTGCAGGCCATGCTCG |
| | | GCCAGAGCACCGAGGAGCTGCGGGTGCGCCTCGCCTCCGCCCTGCGC |
| | | GCCCTGCGTAAGCGGCTCCTCCGCGATGCCGATGACCTGCAGAAGCG |
| | | CCTGGCAGTGTACCAGGCCGGGGCCCGCGAGGGCGCCGAGCGCGGCC |
| | | TCAGCGCCATCCGCGAGCGCCTGGGGCCCCTGGTGGAACAGGGCCGC |
| | | GTGCGGGCCGCCACTGTGGGCTCCCTGGCCGGCCAGCCGCTACAGGA |
| | | GCGGGCCCAGGCCTGGGGCGAGCGGCTGCGCGCGCGGATGGAGGAG |
| | | ATGGGCAGCCGGACCCGCGACCGCCTGGACGAGGTGAAGGAGCAGG |
| | | TGGCGGAGGTGCGCGCCAAGCTGGAGGAGCAGGCCCAGCAGATACG |
| | | CCTGCAGGCCGAGGCCTTCCAGGCCCGCCTCAAGAGCTGGTTCGAGC |
| | | CCCTGGTGGAAGACATGCAGCGCCAGTGGGCCGGGCTGGTGGAGAAG |
| | | GTGCAGGCTGCCGTGGGCACCAGCGCCGCCCCTGTGCCCAGCGACAA |
| | | TCACGGTGGAAGCAGTAGATCATCCAGTTCTGGTGGGGGAGGATCCG |
| | | GGGGGGGGGTGAGGTGCGGCTGCAGCAGTCCGGCCCCGACCTGATC |
| | | AAGCCCGGCGCCTCCGTGAAGATGTCTTGCAAAGCCTCCGGCTACAC |
| | | CTTCACAGGATATGTGATGCACTGGGTGAAGCAGAGGCCCGGCCAGG |
| | | GACTGGAATGGATCGGCTTCATCAACCCATACAACGATGACATTCAG |
| | | TCCAACGAGAGATTTAGGGGAAAGGCCACCCTGACCAGTGACAAATC |
| | | AAGCACTACAGCCTACATGGAGCTGAGCTCACTGACCTCTGAGGACT |
| | | CCGCCGTGTATTACTGTGCCAGAGGCGCCGGCTACAATTTTGACGGCG |
| | | CCTACCGGTTCTTCGACTTCTGGGGGCAGGGCACCACCCTGACCGTGT |
| | | CCAGCTCCCCCAATAGTGCCTCCCATTCTGGCTCCGCCCCCCAGACTA |
| | | GCTCTGCCCCTGGGTCCCAGGACGTGGTGATGACCCAGTCCCCCCTGT |
| | | CTCTGCCTGTGTCCCTGGGAGACCAGGCCTCTATCAGCTGCCGGAGCT |
| | | CTCAGAGGCTGGTGCACAGCAATGGGAATACATACCTGCACTGGTAC |
| | | CTGCAGAAGCCAGGCCAGAGCCCCAAGCTGCTGATCTACAGAGTGAG |
| | | CAACAGGTTCCCCGGAGTCCCTGATAGGTTTAGCGGATCTGGCAGCG |
| | | GCACCGACTTCACTCTGAAGATTAGCAGAGTGGAGGCCGAGGATCTG |
| | | GGCATCTACTTTTGCTCTCAGTCCACCCATGTGCCTTACACCTTTGGG |
| | | GGAGGCACCAAGCTGGAGATTAAGAGGGGCGGGGGCGGCTCCCACC |
| | | ACCACCATCACCACCACCAC |
| CM04K | 32 | CAGATCCAGCTGGTGCAGTCCGGCCCAGAGCTGAAAAAGCCAGGCGA |
| | | GACCGTGAAGATCTCCTGCAAGGCCAGCGGCTACACTTTCAAGAATT |
| | | ATGGCATGAACTGGGTGAAGCAGGCCCCCGGCAAGGGGCTGAAGTG |
| | | GATGGGGTGGATTAACACCTATACCGGCCAGCCCATTTACGCCAACG |
| | | ATTTCAAGGGGAGATTCGCCTTTAGCCTGGAAACCAGCGCGAGCACC |
| | | GCCTATCTGCAGATCAACAATCTTAAGAATGAGGATACAGCCACCTA |
| | | CTTTTGCGCCCGGGATTGGGGCCCATACTGGGGCCAGGGCACCCTGG |
| | | TGATCGTGTCCGCTTCCCCCAATTCTGCCTCCCACAGCGGCAGCGCCC |
| | | CACAGACTAGCTCCGCCCCCGGTTCCCAGAATATCATGATGACACAG |
| | | AGCCCTTCCAGCCTGGCCGTGTCTGCCGGCGAAAAGGTGACCGTGAA |
| | | CTGCAAGTCTAGCCAGTCTGTGCTGTACAGCTCCAATCAGATGAACTA |
| | | TCTGGCCTGGTACCAGCAGAAGCCTGGCCAGAGCCCTAAGCTGCTGA |
| | | TTTACTGGGCCAGCACACGCGAGTCTGGGGTCCCTGACAGATTTACCG |
| | | GTAGCGGAAGCGGCACAGATTTCACACTGACAATCTCTTCTGTGCAG |
| | | ACCGAAGACCTCGCAGTGTACTACTGTCTGCAGTATCTGAGCTCTTGG |
| | | ACTTTCGGGGGGGGACCAAGCTGGAAATTAAGGGTGGAAGCAGTAG |
| | | ATCATCCAGTTCTGGTGGGGGAGGATCCGGGGGCGGGGGTCAGATCC |
| | | AGCTCGTACAAAGTGGTCCAGGTCTCAAAAAACCAGGAGGAAGCGTC |
| | | CGGATAAGCTGCGCCGCTTCAGGATATACCTTTACGAATTACGGTATG |
| | | AATTGGGTAAAACAGGCTCCGGGAAAAGGACTCAGATGGATGGGGT |
| | | GGATCAATACGCACACGGGCGAGCCGACTTACGCGGATGATTTTAAG |
| | | GGTCGCTTTACTTTTTCCCTCGACACCTCAAAATCCACTGCCTATCTGC |
| | | AAATAAACAGTCTTCGCGCAGAGGATACAGCCACCTATTTTTGCACA |
| | | AGGCGGGGGTACGATTGGTATTTCGATGTATGGGGGCAGGGTACAAC |
| | | GGTAACAGTGAGTTCAAGTCCCAACAGCGCCTCTCACAGCGGCAGCG |
| | | CACCTCAGACGAGCTCTGCTCCTGGCAGCCAAGACATCCAGATGACC |
| | | CAGAGCCCTTCCTCTATGTCCGCATCCCTCGGAGACCGCGTAACAATC |
| | | ACATGTCGAGCCAGTCAAGACATCAATAGCTATCTTAGTTGGTTCCAG |
| | | CAGAAACCTGGAAAAAGTCCTAAGACCCTTATTTATAGAGCGAATAG |
| | | GTTGGTTGATGGAGTCCCGTCTAGGTTTAGCGGGTCTGGGTCTGGGAC |
| | | TGACTACACCCTTACTATCTCCTCTCTGCAATACGAAGACTTCGGCAT |
| | | TTATTATTGCCAGCAATATGATGAGTCTCCCTGGACTTTCGGTGGGGG |
| | | GACCAAGTTGGAAATAAAAGGCGGGGGCGGCTCCCACCACCACCATC |
| | | ACCACCACCAC |
| CM04L | 33 | GCTGGACAGCCGCTGCAAGAGCGGGCACAAGCCTGGGGGGAAAGAC |
| | | TCAGGGCCAGAATGGAAGAAATGGGATCAAGAACAAGGGACAGGCT |
| | | CGATGAGGTGAAGGAGCAAGTAGCAGAGGTTCGAGCAAAGCTGGAA |
| | | GAACAAGCTCAGCAGATTAGATTGCAAGCAGAGGCGGCTCAAGCACG |
| | | ACTCAAGAGCAGGTTCGAGCCATTGGCTGAAGATATGCAGCGACAAT |
| | | GGGCTGGTCAGGTTGAGAAAGTCCAAGCGGCGGAAGGGACCAGTGC |
| | | AGCACCGGTACCATCCGACAATCATGGTGGAAGCAGTAGATCATCCA |
| | | GTTCTGGTGGGGGAGGATCCGGGGGCGGGGGTCAGATCCAGCTCGTA |
| | | CAAAGTGGTCCAGGTCTCAAAAAACCAGGAGGAAGCGTCCGGATAA |
| | | GCTGCGCCGCTTCAGGATATACCTTTACGAATTACGGTATGAATTGGG |
| | | TAAAACAGGCTCCGGGAAAAGGACTCAGATGGATGGGGTGGATCAAT |
| | | ACGCACACGGGCGAGCCGACTTACGCGGATGATTTTAAGGGTCGCTT |
| | | TACTTTTTCCCTCGACACCTCAAAATCCACTGCCTATCTGCAAATAAA |
| | | CAGTCTTCGCGCAGAGGATACAGCCACCTATTTTTGCACAAGGCGGG |
| | | GGTACGATTGGTATTTCGATGTATGGGGGCAGGGTACAACGGTAACA |
| | | GTGAGTTCAAGTCCCAACAGCGCCTCTCACAGCGGCAGCGCACCTCA |
| | | GACGAGCTCTGCTCCTGGCAGCCAAGACATCCAGATGACCCAGAGCC |
| | | CTTCCTCTATGTCCGCATCCCTCGGAGACCGCGTAACAATCACATGTC |
| | | GAGCCAGTCAAGACATCAATAGCTATCTTAGTTGGTTCCAGCAGAAA |
| | | CCTGGAAAAAGTCCTAAGACCCTTATTTATAGAGCGAATAGGTTGGTT |
| | | GATGGAGTCCCGTCTAGGTTTAGCGGGTCTGGGTCTGGGACTGACTAC |
| | | ACCCTTACTATCTCCTCTCTGCAATACGAAGACTTCGGCATTTATTATT |
| | | GCCAGCAATATGATGAGTCTCCCTGGACTTTCGGTGGGGGGACCAAG |
| | | TTGGAAATAAAAGGCGGGGGCGGCTCCCACCACCACCATCACCACCA |
| | | CCAC |
| CM04M | 34 | CAGATCCAGCTGGTGCAGTCCGGCCCAGAGCTGAAAAAGCCAGGCGA |
| | | GACCGTGAAGATCTCCTGCAAGGCCAGCGGCTACACTTTCAAGAATT |
| | | ATGGCATGAACTGGGTGAAGCAGGCCCCCGGCAAGGGGCTGAAGTG |
| | | GATGGGGTGGATTAACACCTATACCGGCCAGCCCATTTACGCCAACG |
| | | ATTTCAAGGGGAGATTCGCCTTTAGCCTGGAAACCAGCGCGAGCACC |
| | | GCCTATCTGCAGATCAACAATCTTAAGAATGAGGATACAGCCACCTA |
| | | CTTTTGCGCCCGGGATTGGGGCCCATACTGGGGCCAGGGCACCCTGG |
| | | TGATCGTGTCCGCTTCCCCCAATTCTGCCTCCCACAGCGGCAGCGCCC |
| | | CACAGACTAGCTCCGCCCCCGGTTCCCAGAATATCATGATGACACAG |
| | | AGCCCTTCCAGCCTGGCCGTGTCTGCCGGCGAAAAGGTGACCGTGAA |
| | | CTGCAAGTCTAGCCAGTCTGTGCTGTACAGCTCCAATCAGATGAACTA |
| | | TCTGGCCTGGTACCAGCAGAAGCCTGGCCAGAGCCCTAAGCTGCTGA |
| | | TTTACTGGGCCAGCACACGCGAGTCTGGGGTCCCTGACAGATTTACCG |
| | | GTAGCGGAAGCGGCACAGATTTCACACTGACAATCTCTTCTGTGCAG |
| | | ACCGAAGACCTCGCAGTGTACTACTGTCTGCAGTATCTGAGCTCTTGG |
| | | ACTTTCGGCGGGGGGACCAAGCTGGAAATTAAGGGCGGATCTAGTCG |
| | | GTCATCCTCCAGTGGTGGTGGGGGTAGCGGGGGAGGCGGTGCTGGAC |
| | | AGCCGCTGCAAGAGCGGGCACAAGCCTGGGGGGAAAGACTCAGGGC |
| | | CAGAATGGAAGAAATGGGATCAAGAACAAGGGACAGGCTCGATGAG |
| | | GTGAAGGAGCAAGTAGCAGAGGTTCGAGCAAAGCTGGAAGAACAAG |
| | | CTCAGCAGATTAGATTGCAAGCAGAGGCGGCTCAAGCACGACTCAAG |
| | | AGCAGGTTCGAGCCATTGGCTGAAGATATGCAGCGACAATGGGCTGG |
| | | TCAGGTTGAGAAAGTCCAAGCGGCGGAAGGGACCAGTGCAGCACCG |
| | | GTACCATCCGACAATCATGGTGGAAGCAGTAGATCATCCAGTTCTGG |
| | | TGGGGGAGGATCCGGGGGCGGGGGTCAGATCCAGCTCGTACAAAGTG |
| | | GTCCAGGTCTCAAAAAACCAGGAGGAAGCGTCCGGATAAGCTGCGCC |
| | | GCTTCAGGATATACCTTTACGAATTACGGTATGAATTGGGTAAAACA |
| | | GGCTCCGGGAAAAGGACTCAGATGGATGGGGTGGATCAATACGCACA |
| | | CGGGCGAGCCGACTTACGCGGATGATTTTAAGGGTCGCTTTACTTTTT |
| | | CCCTCGACACCTCAAAATCCACTGCCTATCTGCAAATAAACAGTCTTC |
| | | GCGCAGAGGATACAGCCACCTATTTTTGCACAAGGCGGGGGTACGAT |
| | | TGGTATTTCGATGTATGGGGGCAGGGTACAACGGTAACAGTGAGTTC |
| | | AAGTCCCAACAGCGCCTCTCACAGCGGCAGCGCACCTCAGACGAGCT |
| | | CTGCTCCTGGCAGCCAAGACATCCAGATGACCCAGAGCCCTTCCTCTA |
| | | TGTCCGCATCCCTCGGAGACCGCGTAACAATCACATGTCGAGCCAGT |
| | | CAAGACATCAATAGCTATCTTAGTTGGTTCCAGCAGAAACCTGGAAA |
| | | AAGTCCTAAGACCCTTATTTATAGAGCGAATAGGTTGGTTGATGGAGT |
| | | CCCGTCTAGGTTTAGCGGGTCTGGGTCTGGGACTGACTACACCCTTAC |
| | | TATCTCCTCTCTGCAATACGAAGACTTCGGCATTTATTATTGCCAGCA |
| | | ATATGATGAGTCTCCCTGGACTTTCGGTGGGGGGACCAAGTTGGAAA |
| | | TAAAAGGCGGGGGCGGCTCCCACCACCACCATCACCACCACCAC |
| CM04N | 35 | AAGGTGGAGCAAGCGGTGGAGACAGAGCCGGAGCCCGAGCTGCGCC |
| | | AGCAGACCGAGTGGCAGAGCGGCCAGCGCTGGGAACTGGCACTGGG |
| | | TCGCTTTTGGGATTACCTGCGCTGGGTGCAGACACTGTCTGAGCAGGT |
| | | GCAGGAGGAGCTGCTCAGCTCCCAGGTCACCCAGGAACTGAGGGCGC |
| | | TGATGGACGAGACCATGAAGGAGTTGAAGGCCTACAAATCGGAACTG |
| | | GAGGAACAACTGACCCCGGTGGCGGAGGAGACGCGGGCACGGCTGT |
| | | CCAAGGAGCTGCAGGCGGCGCAGGCCCGGCTGGGCGCGGACATGGA |
| | | GGACGTGTGCGGCCGCCTGGTGCAGTACCGCGGCGAGGTGCAGGCCA |
| | | TGCTCGGCCAGAGCACCGAGGAGCTGCGGGTGCGCCTCGCCTCCGCC |
| | | CTGCGCGCCCTGCGTAAGCGGCTCCTCCGCGATGCCGATGACCTGCA |
| | | GAAGCGCCTGGCAGTGTACCAGGCCGGGGCCCGCGAGGGCGCCGAG |
| | | CGCGGCCTCAGCGCCATCCGCGAGCGCCTGGGGCCCCTGGTGGAACA |
| | | GGGCCGCGTGCGGGCCGCCACTGTGGGCTCCCTGGCCGGCCAGCCGC |
| | | TACAGGAGCGGGCCCAGGCCTGGGGCGAGCGGCTGCGCGCGCGGAT |
| | | GGAGGAGATGGGCAGCCGGACCCGCGACCGCCTGGACGAGGTGAAG |
| | | GAGCAGGTGGCGGAGGTGCGCGCCAAGCTGGAGGAGCAGGCCCAGC |
| | | AGATACGCCTGCAGGCCGAGGCCTTCCAGGCCCGCCTCAAGAGCTGG |
| | | TTCGAGCCCCTGGTGGAAGACATGCAGCGCCAGTGGGCCGGGCTGGT |
| | | GGAGAAGGTGCAGGCTGCCGTGGGCACCAGCGCCGCCCCTGTGCCCA |
| | | GCGACAATCACGGTGGAAGCAGTAGATCATCCAGTTCTGGTGGGGGA |
| | | GGATCCGGGGGCGGGGGTCAGATCCAGCTCGTACAAAGTGGTCCAGG |
| | | TCTCAAAAAACCAGGAGGAAGCGTCCGGATAAGCTGCGCCGCTTCAG |
| | | GATATACCTTTACGAATTACGGTATGAATTGGGTAAAACAGGCTCCG |
| | | GGAAAAGGACTCAGATGGATGGGGTGGATCAATACGCACACGGGCG |
| | | AGCCGACTTACGCGGATGATTTTAAGGGTCGCTTTACTTTTTCCCTCG |
| | | ACACCTCAAAATCCACTGCCTATCTGCAAATAAACAGTCTTCGCGCAG |
| | | AGGATACAGCCACCTATTTTTGCACAAGGCGGGGGTACGATTGGTAT |
| | | TTCGATGTATGGGGGCAGGGTACAACGGTAACAGTGAGTTCAAGTCC |
| | | CAACAGCGCCTCTCACAGCGGCAGCGCACCTCAGACGAGCTCTGCTC |
| | | CTGGCAGCCAAGACATCCAGATGACCCAGAGCCCTTCCTCTATGTCCG |
| | | CATCCCTCGGAGACCGCGTAACAATCACATGTCGAGCCAGTCAAGAC |
| | | ATCAATAGCTATCTTAGTTGGTTCCAGCAGAAACCTGGAAAAAGTCCT |
| | | AAGACCCTTATTTATAGAGCGAATAGGTTGGTTGATGGAGTCCCGTCT |
| | | AGGTTTAGCGGGTCTGGGTCTGGGACTGACTACACCCTTACTATCTCC |
| | | TCTCTGCAATACGAAGACTTCGGCATTTATTATTGCCAGCAATATGAT |
| | | GAGTCTCCCTGGACTTTCGGTGGGGGGACCAAGTTGGAAATAAAAGG |
| | | CGGGGGCGGCTCCCACCACCACCATCACCACCACCAC |
| CM040 | 36 | CAGATCCAGCTGGTGCAGTCCGGCCCAGAGCTGAAAAAGCCAGGCGA |
| | | GACCGTGAAGATCTCCTGCAAGGCCAGCGGCTACACTTTCAAGAATT |
| | | ATGGCATGAACTGGGTGAAGCAGGCCCCCGGCAAGGGGCTGAAGTG |
| | | GATGGGGTGGATTAACACCTATACCGGCCAGCCCATTTACGCCAACG |
| | | ATTTCAAGGGGAGATTCGCCTTTAGCCTGGAAACCAGCGCGAGCACC |
| | | GCCTATCTGCAGATCAACAATCTTAAGAATGAGGATACAGCCACCTA |
| | | CTTTTGCGCCCGGGATTGGGGCCCATACTGGGGCCAGGGCACCCTGG |
| | | TGATCGTGTCCGCTTCCCCCAATTCTGCCTCCCACAGCGGCAGCGCCC |
| | | CACAGACTAGCTCCGCCCCCGGTTCCCAGAATATCATGATGACACAG |
| | | AGCCCTTCCAGCCTGGCCGTGTCTGCCGGCGAAAAGGTGACCGTGAA |
| | | CTGCAAGTCTAGCCAGTCTGTGCTGTACAGCTCCAATCAGATGAACTA |
| | | TCTGGCCTGGTACCAGCAGAAGCCTGGCCAGAGCCCTAAGCTGCTGA |
| | | TTTACTGGGCCAGCACACGCGAGTCTGGGGTCCCTGACAGATTTACCG |
| | | GTAGCGGAAGCGGCACAGATTTCACACTGACAATCTCTTCTGTGCAG |
| | | ACCGAAGACCTCGCAGTGTACTACTGTCTGCAGTATCTGAGCTCTTGG |
| | | ACTTTCGGCGGGGGGACCAAGCTGGAAATTAAGGGCGGATCTAGTCG |
| | | GTCATCCTCCAGTGGTGGTGGGGGTAGCGGGGGAGGCGGTAAGGTGG |
| | | AGCAAGCGGTGGAGACAGAGCCGGAGCCCGAGCTGCGCCAGCAGAC |
| | | CGAGTGGCAGAGCGGCCAGCGCTGGGAACTGGCACTGGGTCGCTTTT |
| | | GGGATTACCTGCGCTGGGTGCAGACACTGTCTGAGCAGGTGCAGGAG |
| | | GAGCTGCTCAGCTCCCAGGTCACCCAGGAACTGAGGGCGCTGATGGA |
| | | CGAGACCATGAAGGAGTTGAAGGCCTACAAATCGGAACTGGAGGAA |
| | | CAACTGACCCCGGTGGCGGAGGAGACGCGGGCACGGCTGTCCAAGG |
| | | AGCTGCAGGCGGCGCAGGCCCGGCTGGGCGCGGACATGGAGGACGT |
| | | GTGCGGCCGCCTGGTGCAGTACCGCGGCGAGGTGCAGGCCATGCTCG |
| | | GCCAGAGCACCGAGGAGCTGCGGGTGCGCCTCGCCTCCGCCCTGCGC |
| | | GCCCTGCGTAAGCGGCTCCTCCGCGATGCCGATGACCTGCAGAAGCG |
| | | CCTGGCAGTGTACCAGGCCGGGGCCCGCGAGGGCGCCGAGCGCGGCC |
| | | TCAGCGCCATCCGCGAGCGCCTGGGGCCCCTGGTGGAACAGGGCCGC |
| | | GTGCGGGCCGCCACTGTGGGCTCCCTGGCCGGCCAGCCGCTACAGGA |
| | | GCGGGCCCAGGCCTGGGGCGAGCGGCTGCGCGCGCGGATGGAGGAG |
| | | ATGGGCAGCCGGACCCGCGACCGCCTGGACGAGGTGAAGGAGCAGG |
| | | TGGCGGAGGTGCGCGCCAAGCTGGAGGAGCAGGCCCAGCAGATACG |
| | | CCTGCAGGCCGAGGCCTTCCAGGCCCGCCTCAAGAGCTGGTTCGAGC |
| | | CCCTGGTGGAAGACATGCAGCGCCAGTGGGCCGGGCTGGTGGAGAAG |
| | | GTGCAGGCTGCCGTGGGCACCAGCGCCGCCCCTGTGCCCAGCGACAA |
| | | TCACGGTGGAAGCAGTAGATCATCCAGTTCTGGTGGGGGAGGATCCG |
| | | GGGGCGGGGGTCAGATCCAGCTCGTACAAAGTGGTCCAGGTCTCAAA |
| | | AAACCAGGAGGAAGCGTCCGGATAAGCTGCGCCGCTTCAGGATATAC |
| | | CTTTACGAATTACGGTATGAATTGGGTAAAACAGGCTCCGGGAAAAG |
| | | GACTCAGATGGATGGGGTGGATCAATACGCACACGGGCGAGCCGACT |
| | | TACGCGGATGATTTTAAGGGTCGCTTTACTTTTTCCCTCGACACCTCA |
| | | AAATCCACTGCCTATCTGCAAATAAACAGTCTTCGCGCAGAGGATAC |
| | | AGCCACCTATTTTTGCACAAGGCGGGGGTACGATTGGTATTTCGATGT |
| | | ATGGGGGCAGGGTACAACGGTAACAGTGAGTTCAAGTCCCAACAGCG |
| | | CCTCTCACAGCGGCAGCGCACCTCAGACGAGCTCTGCTCCTGGCAGC |
| | | CAAGACATCCAGATGACCCAGAGCCCTTCCTCTATGTCCGCATCCCTC |
| | | GGAGACCGCGTAACAATCACATGTCGAGCCAGTCAAGACATCAATAG |
| | | CTATCTTAGTTGGTTCCAGCAGAAACCTGGAAAAAGTCCTAAGACCCT |
| | | TATTTATAGAGCGAATAGGTTGGTTGATGGAGTCCCGTCTAGGTTTAG |
| | | CGGGTCTGGGTCTGGGACTGACTACACCCTTACTATCTCCTCTCTGCA |
| | | ATACGAAGACTTCGGCATTTATTATTGCCAGCAATATGATGAGTCTCC |
| | | CTGGACTTTCGGTGGGGGGACCAAGTTGGAAATAAAAGGCGGGGGCG |
| | | GCTCCCACCACCACCATCACCACCACCAC |
| CM04P | 37 | CAGATCCAGCTGGTGCAGTCCGGCCCAGAGCTGAAAAAGCCAGGCGA |
| | | GACCGTGAAGATCTCCTGCAAGGCCAGCGGCTACACTTTCAAGAATT |
| | | ATGGCATGAACTGGGTGAAGCAGGCCCCCGGCAAGGGGCTGAAGTG |
| | | GATGGGGTGGATTAACACCTATACCGGCCAGCCCATTTACGCCAACG |
| | | ATTTCAAGGGGAGATTCGCCTTTAGCCTGGAAACCAGCGCGAGCACC |
| | | GCCTATCTGCAGATCAACAATCTTAAGAATGAGGATACAGCCACCTA |
| | | CTTTTGCGCCCGGGATTGGGGCCCATACTGGGGCCAGGGCACCCTGG |
| | | TGATCGTGTCCGCTTCCCCCAATTCTGCCTCCCACAGCGGCAGCGCCC |
| | | CACAGACTAGCTCCGCCCCCGGTTCCCAGAATATCATGATGACACAG |
| | | AGCCCTTCCAGCCTGGCCGTGTCTGCCGGCGAAAAGGTGACCGTGAA |
| | | CTGCAAGTCTAGCCAGTCTGTGCTGTACAGCTCCAATCAGATGAACTA |
| | | TCTGGCCTGGTACCAGCAGAAGCCTGGCCAGAGCCCTAAGCTGCTGA |
| | | TTTACTGGGCCAGCACACGCGAGTCTGGGGTCCCTGACAGATTTACCG |
| | | GTAGCGGAAGCGGCACAGATTTCACACTGACAATCTCTTCTGTGCAG |
| | | ACCGAAGACCTCGCAGTGTACTACTGTCTGCAGTATCTGAGCTCTTGG |
| | | ACTTTCGGCGGGGGGACCAAGCTGGAAATTAAGGGTGGAAGCAGTAG |
| | | ATCATCCAGTAGTGGGGGGGGCGGATCTGGAGGTGGGGGACAGGTG |
| | | AAACTGCAGCAGTCTGGCCCCGAGCTGAAGAAGCCCGGCGAGACCGT |
| | | GAAGATCTCCTGCAAGGCCTCCGGCTACACCTTTACCAATTACGGAAT |
| | | GAACTGGGTGAAGCAGGCTCCAGGAAAGGGCCTGAAATGGATGGGC |
| | | TGGATCAACACTTATACTGGCGAGAGCACCTACGCAGACGATTTCAA |
| | | GGGTAGGTTCGCATTTAGCCTGGAGACCTCCGCCAGCGCCGCCTACCT |
| | | GCAGATCAATAATCTGAAAAACGAGGACACAGCCACCTATTTCTGCG |
| | | CCCGGTTTGCCATCAAAGGGGATTACTGGGGCCAGGGCACCACAGTG |
| | | ACCGTGTCCTCCTCCCCCAATAGTGCCTCCCATTCTGGCTCCGCCCCC |
| | | CAGACTAGCTCTGCCCCTGGGTCCCAGGATATTGTCCTGACCCAGTCC |
| | | CCATTCTCCAACCCCGTGACCCTGGGGACCAGCGCCTCCATTAGCTGT |
| | | AGGTCAACTAAGAGCCTGCTGCACAGCAATGGAATCACCTACCTGTA |
| | | CTGGTACCTGCAGAAGCCAGGCCAGTCCCCTCAGCTGCTGATCTACCA |
| | | GATGAGCAACCTGGCCTCAGGCGTGCCTGATCGGTTTTCCAGCTCCGG |
| | | CAGCGGCACCGACTTCACCCTGCGGATCTCCAGGGTGGAGGCTGAAG |
| | | ACGTGGGGGTGTACTACTGCGCCCAGAACCTGGAGATCCCTAGGACA |
| | | TTTGGAGGCGGCACCAAGCTGGAGATCAAGGGCGGGGGCGGCTCCCA |
| | | CCACCACCATCACCACCACCAC |

TABLE 5
Amino Acid Sequences of Domains of Multi-Specific Reagent Constructs

| | SEQ ID | |
|---|---|---|
| Construct: | NO: | Sequence: |
| Anti-PEG (scFv) | 38 | QIQLVQSGPELKKPGETVKISCKASGYTFKNYGMNWVKQAPGKGLK |
| | | WMGWINTYTGQPIYANDFKGRFAFSLETSASTAYLQINNLKNEDTAT |
| | | YFCARDWGPYWGQGTLVIVSASPNSASHSGSAPQTSSAPGSQNIMM |
| | | TQSPSSLAVSAGEKVTVNCKSSQSVLYSSNQMNYLAWYQQKPGQSP |
| | | KLLIYWASTRESGVPDRFTGSGSGTDFTLTISSVQTEDLAVYYCLQYL |
| | | SSWTFGGGTKLEIK |
| Linker 2 | 2 | GGSSRSSSSGGGGSGGGG |
| Anti-CD3 (scFv) | 39 | EVQLLESGGGLVQPGGSLRLSCAASGFTFSSFPMAWVRQAPGKGLE |
| | | WVSTISTSGGRTYYRDSVKGRFTISRDNSKNTLYLQMNSLRAEDTAV |
| | | YYCAKFRQYSGGFDYWGQGTLVTVSSSPNSASHSGSAPQTSSAPGSQ |
| | | DIQLTQPNSVSTSLGSTVKLSCTLSSGNIENNYVHWYQLYEGRSPTT |
| | | MIYDDDKRPDGVPDRFSGSIDRSSNSAFLTIHNVAIEDEAIYFCHSYVS |
| | | SFNVFGGGTKLTVLR |
| Mutated ApoE3 | 40 | AGQPLQERAQAWGERLRARMEEMGSRTRDRLDEVKEQVAEVRAKL |
| LBD (lipid | | EEQAQQIRLQAEAAQARLKSRFEPLAEDMQRQWAGQVEKVQAAEG |
| binding domain) | | TSAAPVPSDNH |
| Mutated ApoE3 | 41 | KVEQAVETEPEPELRQQTEWQSGQRWELALGRFWDYLRWVQTLSE |
| FL (full length) | | QVQEELLSSQVTQELRALMDETMKELKAYKSELEEQLTPVAEETRA |
| | | RLSKELQAAQARLGADMEDVCGRLVQYRGEVQAMLGQSTEELRVR |
| | | LASALRALRKRLLRDADDLQKRLAVYQAGAREGAERGLSAIRERLG |
| | | PLVEQGRVRAATVGSLAGQPLQERAQAWGERLRARMEEMGSRTRD |
| | | RLDEVKEQVAEVRAKLEEQAQQIRLQAEAFQARLKSWFEPLVEDMQ |
| | | RQWAGLVEKVQAAVGTSAAPVPSDNH |
| Anti-TRBC1 | 42 | EVRLQQSGPDLIKPGASVKMSCKASGYTFTGYVMHWVKQRPGQGL |
| (scFv) | | EWIGFINPYNDDIQSNERFRGKATLTSDKSSTTAYMELSSLTSEDSAV |
| | | YYCARGAGYNFDGAYRFFDFWGQGTTLTVSSSPNSASHSGSAPQTSS |
| | | APGSQDVVMTQSPLSLPVSLGDQASISCRSSQRLVHSNGNTYLHWYL |
| | | QKPGQSPKLLIYRVSNRFPGVPDRFSGSGSGTDFTLKISRVEAEDLGIY |
| | | FCSQSTHVPYTFGGGTKLEIK |
| Anti-CD5 (scFv) | 43 | QIQLVQSGPGLKKPGGSVRISCAASGYTFTNYGMNWVKQAPGKGLR |
| | | WMGWINTHTGEPTYADDFKGRFTFSLDTSKSTAYLQINSLRAEDTAT |
| | | YFCTRRGYDWYFDVWGQGTTVTVSSSPNSASHSGSAPQTSSAPGSQ |
| | | DIQMTQSPSSMSASLGDRVTITCRASQDINSYLSWFQQKPGKSPKTLI |
| | | YRANRLVDGVPSRFSGSGSGTDYTLTISSLQYEDFGIYYCQQYDESP |
| | | WTFGGGTKLEIK |
| Anti- | 44 | QVKLQQSGPELKKPGETVKISCKASGYTFTNYGMNWVKQAPGKGL |
| EpCAM(scFv) | | KWMGWINTYTGESTYADDFKGRFAFSLETSASAAYLQINNLKNEDT |
| | | ATYFCARFAIKGDYWGQGTTVTVSSSPNSASHSGSAPQTSSAPGSQDI |
| | | VLTQSPFSNPVTLGTSASISCRSTKSLLHSNGITYLYWYLQKPGQSPQ |
| | | LLIYQMSNLASGVPDRESSSGSGTDFTLRISRVEAEDVGVYYCAQNL |
| | | EIPRTFGGGTKLEIK |

TABLE 6
DNA Sequences of Domains of Multi-Specific Reagent Constructs

| | SEQ ID | |
|---|---|---|
| Construct: | NO: | Sequence: |
| anti-PEG (scFv) | 45 | CAGATCCAGCTGGTGCAGTCCGGCCCAGAGCTGAAAAAGCCAGG |
| | | CGAGACCGTGAAGATCTCCTGCAAGGCCAGCGGCTACACTTTCAA |
| | | GAATTATGGCATGAACTGGGTGAAGCAGGCCCCCGGCAAGGGGC |
| | | TGAAGTGGATGGGGTGGATTAACACCTATACCGGCCAGCCCATTT |
| | | ACGCCAACGATTTCAAGGGGAGATTCGCCTTTAGCCTGGAAACCA |
| | | GCGCGAGCACCGCCTATCTGCAGATCAACAATCTTAAGAATGAGG |
| | | ATACAGCCACCTACTTTTGCGCCCGGGATTGGGGCCCATACTGGG |
| | | GCCAGGGCACCCTGGTGATCGTGTCCGCTTCCCCCAATTCTGCCTC |
| | | CCACAGCGGCAGCGCCCCACAGACTAGCTCCGCCCCCGGTTCCCA |
| | | GAATATCATGATGACACAGAGCCCTTCCAGCCTGGCCGTGTCTGC |
| | | CGGCGAAAAGGTGACCGTGAACTGCAAGTCTAGCCAGTCTGTGCT |
| | | GTACAGCTCCAATCAGATGAACTATCTGGCCTGGTACCAGCAGAA |
| | | GCCTGGCCAGAGCCCTAAGCTGCTGATTTACTGGGCCAGCACACG |
| | | CGAGTCTGGGGTCCCTGACAGATTTACCGGTAGCGGAAGCGGCAC |
| | | AGATTTCACACTGACAATCTCTTCTGTGCAGACCGAAGACCTCGC |
| | | AGTGTACTACTGTCTGCAGTATCTGAGCTCTTGGACTTTCGGCGGG |
| | | GGGACCAAGCTGGAAATTAAG |
| Linker 2 | 46 | GGTGGAAGCAGTAGATCATCCAGTTCTGGTGGGGGAGGATCCGG |
| | | GGGCGGGGGT |
| Anti-CD3 (scFv) | 47 | GAGGTTCAGCTCCTTGAATCAGGCGGAGGGCTCGTCCAACCAGGC |
| | | GGATCTTTGCGGCTGTCATGTGCCGCATCCGGATTCACGTTCTCCT |
| | | CCTTTCCGATGGCATGGGTGAGGCAAGCTCCTGGAAAAGGTCTCG |
| | | AGTGGGTATCCACCATAAGTACTTCTGGCGGGCGCACTTACTACA |
| | | GGGATAGTGTGAAGGGAAGGTTTACCATTTCCAGAGATAACTCAA |
| | | AGAATACCCTGTACCTGCAAATGAACTCCCTCAGGGCTGAAGATA |
| | | CGGCTGTGTACTACTGTGCTAAATTTCGACAATACTCAGGTGGGT |
| | | TTGACTATTGGGGCCAGGGGACACTGGTGACTGTGTCATCAAGTC |
| | | CCAATTCCGCCTCACATAGCGGAAGTGCCCCACAAACTTCCTCCG |
| | | CGCCAGGTTCTCAAGACATTCAATTGACGCAGCCGAATAGCGTCT |
| | | CTACGAGCTTGGGGAGTACCGTTAAGCTTTCATGTACTCTTTCCTC |
| | | CGGTAACATAGAGAACAACTACGTGCATTGGTACCAACTCTACGA |
| | | GGGCAGGTCTCCAACGACTATGATTTACGATGACGATAAACGACC |
| | | TGATGGGGTCCCTGACCGCTTTTCAGGAAGCATTGACAGAAGCTC |
| | | TAATTCCGCGTTTTTGACTATCCACAACGTAGCGATTGAAGATGA |
| | | AGCAATTTACTTCTGTCATTCCTATGTATCCAGCTTTAACGTCTTC |
| | | GGAGGTGGAACTAAGTTGACGGTACTCCGA |
| Mutated ApoE3 | 48 | GCTGGACAGCCGCTGCAAGAGCGGGCACAAGCCTGGGGGGAAAG |
| LBD (lipid | | ACTCAGGGCCAGAATGGAAGAAATGGGATCAAGAACAAGGGACA |
| binding domain) | | GGCTCGATGAGGTGAAGGAGCAAGTAGCAGAGGTTCGAGCAAAG |
| | | CTGGAAGAACAAGCTCAGCAGATTAGATTGCAAGCAGAGGCGGC |
| | | TCAAGCACGACTCAAGAGCAGGTTCGAGCCATTGGCTGAAGATAT |
| | | GCAGCGACAATGGGCTGGTCAGGTTGAGAAAGTCCAAGCGGCGG |
| | | AAGGGACCAGTGCAGCACCGGTACCATCCGACAATCAT |
| Mutated ApoE3 | 49 | AAGGTGGAGCAAGCGGTGGAGACAGAGCCGGAGCCCGAGCTGCG |
| FL (full length) | | CCAGCAGACCGAGTGGCAGAGCGGCCAGCGCTGGGAACTGGCAC |
| | | TGGGTCGCTTTTGGGATTACCTGCGCTGGGTGCAGACACTGTCTG |
| | | AGCAGGTGCAGGAGGAGCTGCTCAGCTCCCAGGTCACCCAGGAA |
| | | CTGAGGGCGCTGATGGACGAGACCATGAAGGAGTTGAAGGCCTA |
| | | CAAATCGGAACTGGAGGAACAACTGACCCCGGTGGCGGAGGAGA |
| | | CGCGGGCACGGCTGTCCAAGGAGCTGCAGGCGGCGCAGGCCCGG |
| | | CTGGGCGCGGACATGGAGGACGTGTGCGGCCGCCTGGTGCAGTA |
| | | CCGCGGCGAGGTGCAGGCCATGCTCGGCCAGAGCACCGAGGAGC |
| | | TGCGGGTGCGCCTCGCCTCCGCCCTGCGCGCCCTGCGTAAGCGGC |
| | | TCCTCCGCGATGCCGATGACCTGCAGAAGCGCCTGGCAGTGTACC |
| | | AGGCCGGGGCCCGCGAGGGCGCCGAGCGCGGCCTCAGCGCCATC |
| | | CGCGAGCGCCTGGGGCCCCTGGTGGAACAGGGCCGCGTGCGGGC |
| | | CGCCACTGTGGGCTCCCTGGCCGGCCAGCCGCTACAGGAGCGGGC |
| | | CCAGGCCTGGGGCGAGCGGCTGCGCGCGCGGATGGAGGAGATGG |
| | | GCAGCCGGACCCGCGACCGCCTGGACGAGGTGAAGGAGCAGGTG |
| | | GCGGAGGTGCGCGCCAAGCTGGAGGAGCAGGCCCAGCAGATACG |
| | | CCTGCAGGCCGAGGCCTTCCAGGCCCGCCTCAAGAGCTGGTTCGA |
| | | GCCCCTGGTGGAAGACATGCAGCGCCAGTGGGCCGGGCTGGTGG |
| | | AGAAGGTGCAGGCTGCCGTGGGCACCAGCGCCGCCCCTGTGCCCA |
| | | GCGACAATCAC |
| Anti-TRBC1 | 50 | GAGGTGCGGCTGCAGCAGTCCGGCCCCGACCTGATCAAGCCCGGC |
| (scFv) | | GCCTCCGTGAAGATGTCTTGCAAAGCCTCCGGCTACACCTTCACA |
| | | GGATATGTGATGCACTGGGTGAAGCAGAGGCCCGGCCAGGGACT |
| | | GGAATGGATCGGCTTCATCAACCCATACAACGATGACATTCAGTC |
| | | CAACGAGAGATTTAGGGGAAAGGCCACCCTGACCAGTGACAAAT |
| | | CAAGCACTACAGCCTACATGGAGCTGAGCTCACTGACCTCTGAGG |
| | | ACTCCGCCGTGTATTACTGTGCCAGAGGCGCCGGCTACAATTTTG |
| | | ACGGCGCCTACCGGTTCTTCGACTTCTGGGGGCAGGGCACCACCC |
| | | TGACCGTGTCCAGCTCCCCCAATAGTGCCTCCCATTCTGGCTCCGC |
| | | CCCCCAGACTAGCTCTGCCCCTGGGTCCCAGGACGTGGTGATGAC |
| | | CCAGTCCCCCCTGTCTCTGCCTGTGTCCCTGGGAGACCAGGCCTCT |
| | | ATCAGCTGCCGGAGCTCTCAGAGGCTGGTGCACAGCAATGGGAAT |
| | | ACATACCTGCACTGGTACCTGCAGAAGCCAGGCCAGAGCCCCAA |
| | | GCTGCTGATCTACAGAGTGAGCAACAGGTTCCCCGGAGTCCCTGA |
| | | TAGGTTTAGCGGATCTGGCAGCGGCACCGACTTCACTCTGAAGAT |
| | | TAGCAGAGTGGAGGCCGAGGATCTGGGCATCTACTTTTGCTCTCA |
| | | GTCCACCCATGTGCCTTACACCTTTGGGGGAGGCACCAAGCTGGA |
| | | GATTAAGAGG |
| Anti-CD5 (scFv) | 51 | CAGATCCAGCTCGTACAAAGTGGTCCAGGTCTCAAAAAACCAGG |
| | | AGGAAGCGTCCGGATAAGCTGCGCCGCTTCAGGATATACCTTTAC |
| | | GAATTACGGTATGAATTGGGTAAAACAGGCTCCGGGAAAAGGAC |
| | | TCAGATGGATGGGGTGGATCAATACGCACACGGGCGAGCCGACT |
| | | TACGCGGATGATTTTAAGGGTCGCTTTACTTTTTCCCTCGACACCT |
| | | CAAAATCCACTGCCTATCTGCAAATAAACAGTCTTCGCGCAGAGG |
| | | ATACAGCCACCTATTTTTGCACAAGGCGGGGGTACGATTGGTATT |
| | | TCGATGTATGGGGGCAGGGTACAACGGTAACAGTGAGTTCAAGTC |
| | | CCAACAGCGCCTCTCACAGCGGCAGCGCACCTCAGACGAGCTCTG |
| | | CTCCTGGCAGCCAAGACATCCAGATGACCCAGAGCCCTTCCTCTA |
| | | TGTCCGCATCCCTCGGAGACCGCGTAACAATCACATGTCGAGCCA |
| | | GTCAAGACATCAATAGCTATCTTAGTTGGTTCCAGCAGAAACCTG |
| | | GAAAAAGTCCTAAGACCCTTATTTATAGAGCGAATAGGTTGGTTG |
| | | ATGGAGTCCCGTCTAGGTTTAGCGGGTCTGGGTCTGGGACTGACT |
| | | ACACCCTTACTATCTCCTCTCTGCAATACGAAGACTTCGGCATTTA |
| | | TTATTGCCAGCAATATGATGAGTCTCCCTGGACTTTCGGTGGGGG |
| | | GACCAAGTTGGAAATAAAA |
| Anti-EpCAM5 | 52 | CAGGTGAAACTGCAGCAGTCTGGCCCCGAGCTGAAGAAGCCCGG |
| (scFv) | | CGAGACCGTGAAGATCTCCTGCAAGGCCTCCGGCTACACCTTTAC |
| | | CAATTACGGAATGAACTGGGTGAAGCAGGCTCCAGGAAAGGGCC |
| | | TGAAATGGATGGGCTGGATCAACACTTATACTGGCGAGAGCACCT |
| | | ACGCAGACGATTTCAAGGGTAGGTTCGCATTTAGCCTGGAGACCT |
| | | CCGCCAGCGCCGCCTACCTGCAGATCAATAATCTGAAAAACGAGG |
| | | ACACAGCCACCTATTTCTGCGCCCGGTTTGCCATCAAAGGGGATT |
| | | ACTGGGGCCAGGGCACCACAGTGACCGTGTCCTCCTCCCCCAATA |
| | | GTGCCTCCCATTCTGGCTCCGCCCCCCAGACTAGCTCTGCCCCTGG |
| | | GTCCCAGGATATTGTCCTGACCCAGTCCCCATTCTCCAACCCCGTG |
| | | ACCCTGGGGACCAGCGCCTCCATTAGCTGTAGGTCAACTAAGAGC |
| | | CTGCTGCACAGCAATGGAATCACCTACCTGTACTGGTACCTGCAG |
| | | AAGCCAGGCCAGTCCCCTCAGCTGCTGATCTACCAGATGAGCAAC |
| | | CTGGCCTCAGGCGTGCCTGATCGGTTTTCCAGCTCCGGCAGCGGC |
| | | ACCGACTTCACCCTGCGGATCTCCAGGGTGGAGGCTGAAGACGTG |
| | | GGGGTGTACTACTGCGCCCAGAACCTGGAGATCCCTAGGACATTT |
| | | GGAGGCGGCACCAAGCTGGAGATCAAG |
| IL-2 signal | 54 | ATGTACAGGATGCAACTCCTGTCTTGCATTGCACTAAGTCTTGCAC |
| sequence | | TTGTCACGAATTCG |

TABLE 7
Amino Acid Sequences of Constructs Comprising the IL-2 Signal Peptide

| | SEQ ID | |
|---|---|---|
| Construct: | NO: | Sequence: |
| CM04A + SP | 55 | MYRMQLLSCIALSLALVTNSQIQLVQSGPELKKPGETVKISCKASGYTFK |
| | | NYGMNWVKQAPGKGLKWMGWINTYTGQPIYANDFKGRFAFSLETSAS |
| | | TAYLQINNLKNEDTATYFCARDWGPYWGQGTLVIVSASPNSASHSGSAP |
| | | QTSSAPGSQNIMMTQSPSSLAVSAGEKVTVNCKSSQSVLYSSNQMNYLA |
| | | WYQQKPGQSPKLLIYWASTRESGVPDRFTGSGSGTDFTLTISSVQTEDLA |
| | | VYYCLQYLSSWTFGGGTKLEIKGGSSRSSSSGGGGSGGGGEVQLLESGG |
| | | GLVQPGGSLRLSCAASGFTFSSFPMAWVRQAPGKGLEWVSTISTSGGRT |
| | | YYRDSVKGRFTISRDNSKNTLYLQMNSLRAEDTAVYYCAKFRQYSGGF |
| | | DYWGQGTLVTVSSSPNSASHSGSAPQTSSAPGSQDIQLTQPNSVSTSLGS |
| | | TVKLSCTLSSGNIENNYVHWYQLYEGRSPTTMIYDDDKRPDGVPDRFSG |
| | | SIDRSSNSAFLTIHNVAIEDEAIYFCHSYVSSFNVFGGGTKLTVLRGGGGS |
| | | HHHHHHHH |
| CM04B + SP | 56 | MYRMQLLSCIALSLALVTNSAGQPLQERAQAWGERLRARMEEMGSRTR |
| | | DRLDEVKEQVAEVRAKLEEQAQQIRLQAEAAQARLKSRFEPLAEDMQR |
| | | QWAGQVEKVQAAEGTSAAPVPSDNHGGSSRSSSSGGGGSGGGGEVQLL |
| | | ESGGGLVQPGGSLRLSCAASGFTFSSFPMAWVRQAPGKGLEWVSTISTS |
| | | GGRTYYRDSVKGRFTISRDNSKNTLYLQMNSLRAEDTAVYYCAKFRQY |
| | | SGGFDYWGQGTLVTVSSSPNSASHSGSAPQTSSAPGSQDIQLTQPNSVST |
| | | SLGSTVKLSCTLSSGNIENNYVHWYQLYEGRSPTTMIYDDDKRPDGVPD |
| | | RFSGSIDRSSNSAFLTIHNVAIEDEAIYFCHSYVSSFNVFGGGTKLTVLRG |
| | | GGGSHHHHHHHH |
| CM04C + SP | 57 | MYRMQLLSCIALSLALVTNSQIQLVQSGPELKKPGETVKISCKASGYTFK |
| | | NYGMNWVKQAPGKGLKWMGWINTYTGQPIYANDFKGRFAFSLETSAS |
| | | TAYLQINNLKNEDTATYFCARDWGPYWGQGTLVIVSASPNSASHSGSAP |
| | | QTSSAPGSQNIMMTQSPSSLAVSAGEKVTVNCKSSQSVLYSSNQMNYLA |
| | | WYQQKPGQSPKLLIYWASTRESGVPDRFTGSGSGTDFTLTISSVQTEDLA |
| | | VYYCLQYLSSWTFGGGTKLEIKGGSSRSSSSGGGGSGGGGAGQPLQERA |
| | | QAWGERLRARMEEMGSRTRDRLDEVKEQVAEVRAKLEEQAQQIRLQAE |
| | | AAQARLKSRFEPLAEDMQRQWAGQVEKVQAAEGTSAAPVPSDNHGGSS |
| | | RSSSSGGGGSGGGGEVQLLESGGGLVQPGGSLRLSCAASGFTFSSFPMA |
| | | WVRQAPGKGLEWVSTISTSGGRTYYRDSVKGRFTISRDNSKNTLYLQMN |
| | | SLRAEDTAVYYCAKFRQYSGGFDYWGQGTLVTVSSSPNSASHSGSAPQT |
| | | SSAPGSQDIQLTQPNSVSTSLGSTVKLSCTLSSGNIENNYVHWYQLYEGR |
| | | SPTTMIYDDDKRPDGVPDRFSGSIDRSSNSAFLTIHNVAIEDEAIYFCHSY |
| | | VSSFNVFGGGTKLTVLRGGGGSHHHHHHHH |

TABLE 8
DNA Sequences of Constructs Comprising the IL-2 Signal Peptide

| | SEQ ID | |
|---|---|---|
| Construct: | NO: | Sequence: |
| CM04A + SP | 58 | ATGTACAGGATGCAACTCCTGTCTTGCATTGCACTAAGTCTTGCACTT |
| | | GTCACGAATTCGCAGATCCAGCTGGTGCAGTCCGGCCCAGAGCTGAA |
| | | AAAGCCAGGCGAGACCGTGAAGATCTCCTGCAAGGCCAGCGGCTACA |
| | | CTTTCAAGAATTATGGCATGAACTGGGTGAAGCAGGCCCCCGGCAAG |
| | | GGGCTGAAGTGGATGGGGTGGATTAACACCTATACCGGCCAGCCCAT |
| | | TTACGCCAACGATTTCAAGGGGAGATTCGCCTTTAGCCTGGAAACCA |
| | | GCGCGAGCACCGCCTATCTGCAGATCAACAATCTTAAGAATGAGGAT |
| | | ACAGCCACCTACTTTTGCGCCCGGGATTGGGGCCCATACTGGGGCCA |
| | | GGGCACCCTGGTGATCGTGTCCGCTTCCCCCAATTCTGCCTCCCACAG |
| | | CGGCAGCGCCCCACAGACTAGCTCCGCCCCCGGTTCCCAGAATATCA |
| | | TGATGACACAGAGCCCTTCCAGCCTGGCCGTGTCTGCCGGCGAAAAG |
| | | GTGACCGTGAACTGCAAGTCTAGCCAGTCTGTGCTGTACAGCTCCAAT |
| | | CAGATGAACTATCTGGCCTGGTACCAGCAGAAGCCTGGCCAGAGCCC |
| | | TAAGCTGCTGATTTACTGGGCCAGCACACGCGAGTCTGGGGTCCCTG |
| | | ACAGATTTACCGGTAGCGGAAGCGGCACAGATTTCACACTGACAATC |
| | | TCTTCTGTGCAGACCGAAGACCTCGCAGTGTACTACTGTCTGCAGTAT |
| | | CTGAGCTCTTGGACTTTCGGCGGGGGGACCAAGCTGGAAATTAAGGG |
| | | TGGAAGCAGTAGATCATCCAGTTCTGGTGGGGGAGGATCCGGGGGCG |
| | | GGGGTGAGGTTCAGCTCCTTGAATCAGGCGGAGGGCTCGTCCAACCA |
| | | GGCGGATCTTTGCGGCTGTCATGTGCCGCATCCGGATTCACGTTCTCC |
| | | TCCTTTCCGATGGCATGGGTGAGGCAAGCTCCTGGAAAAGGTCTCGA |
| | | GTGGGTATCCACCATAAGTACTTCTGGCGGGCGCACTTACTACAGGG |
| | | ATAGTGTGAAGGGAAGGTTTACCATTTCCAGAGATAACTCAAAGAAT |
| | | ACCCTGTACCTGCAAATGAACTCCCTCAGGGCTGAAGATACGGCTGT |
| | | GTACTACTGTGCTAAATTTCGACAATACTCAGGTGGGTTTGACTATTG |
| | | GGGCCAGGGGACACTGGTGACTGTGTCATCAAGTCCCAATTCCGCCT |
| | | CACATAGCGGAAGTGCCCCACAAACTTCCTCCGCGCCAGGTTCTCAA |
| | | GACATTCAATTGACGCAGCCGAATAGCGTCTCTACGAGCTTGGGGAG |
| | | TACCGTTAAGCTTTCATGTACTCTTTCCTCCGGTAACATAGAGAACAA |
| | | CTACGTGCATTGGTACCAACTCTACGAGGGCAGGTCTCCAACGACTAT |
| | | GATTTACGATGACGATAAACGACCTGATGGGGTCCCTGACCGCTTTTC |
| | | AGGAAGCATTGACAGAAGCTCTAATTCCGCGTTTTTGACTATCCACAA |
| | | CGTAGCGATTGAAGATGAAGCAATTTACTTCTGTCATTCCTATGTATC |
| | | CAGCTTTAACGTCTTCGGAGGTGGAACTAAGTTGACGGTACTCCGAG |
| | | GGGGAGGTGGGTCTCACCACCATCACCACCACCATCAT |
| CM04B + SP | 59 | ATGTACAGGATGCAACTCCTGTCTTGCATTGCACTAAGTCTTGCACTT |
| | | GTCACGAATTCGGCTGGACAGCCGCTGCAAGAGCGGGCACAAGCCTG |
| | | GGGGGAAAGACTCAGGGCCAGAATGGAAGAAATGGGATCAAGAACA |
| | | AGGGACAGGCTCGATGAGGTGAAGGAGCAAGTAGCAGAGGTTCGAG |
| | | CAAAGCTGGAAGAACAAGCTCAGCAGATTAGATTGCAAGCAGAGGC |
| | | GGCTCAAGCACGACTCAAGAGCAGGTTCGAGCCATTGGCTGAAGATA |
| | | TGCAGCGACAATGGGCTGGTCAGGTTGAGAAAGTCCAAGCGGCGGAA |
| | | GGGACCAGTGCAGCACCGGTACCATCCGACAATCATGGTGGAAGCAG |
| | | TAGATCATCCAGTTCTGGTGGGGGAGGATCCGGGGGCGGGGGTGAGG |
| | | TTCAGCTCCTTGAATCAGGCGGAGGGCTCGTCCAACCAGGCGGATCTT |
| | | TGCGGCTGTCATGTGCCGCATCCGGATTCACGTTCTCCTCCTTTCCGAT |
| | | GGCATGGGTGAGGCAAGCTCCTGGAAAAGGTCTCGAGTGGGTATCCA |
| | | CCATAAGTACTTCTGGCGGGCGCACTTACTACAGGGATAGTGTGAAG |
| | | GGAAGGTTTACCATTTCCAGAGATAACTCAAAGAATACCCTGTACCT |
| | | GCAAATGAACTCCCTCAGGGCTGAAGATACGGCTGTGTACTACTGTG |
| | | CTAAATTTCGACAATACTCAGGTGGGTTTGACTATTGGGGCCAGGGG |
| | | ACACTGGTGACTGTGTCATCAAGTCCCAATTCCGCCTCACATAGCGGA |
| | | AGTGCCCCACAAACTTCCTCCGCGCCAGGTTCTCAAGACATTCAATTG |
| | | ACGCAGCCGAATAGCGTCTCTACGAGCTTGGGGAGTACCGTTAAGCT |
| | | TTCATGTACTCTTTCCTCCGGTAACATAGAGAACAACTACGTGCATTG |
| | | GTACCAACTCTACGAGGGCAGGTCTCCAACGACTATGATTTACGATG |
| | | ACGATAAACGACCTGATGGGGTCCCTGACCGCTTTTCAGGAAGCATT |
| | | GACAGAAGCTCTAATTCCGCGTTTTTGACTATCCACAACGTAGCGATT |
| | | GAAGATGAAGCAATTTACTTCTGTCATTCCTATGTATCCAGCTTTAAC |
| | | GTCTTCGGAGGTGGAACTAAGTTGACGGTACTCCGAGGGGGAGGTGG |
| | | GTCTCACCACCATCACCACCACCATCAT |
| CM04C + SP | 60 | ATGTACAGGATGCAACTCCTGTCTTGCATTGCACTAAGTCTTGCACTT |
| | | GTCACGAATTCGCAGATCCAGCTGGTGCAGTCCGGCCCAGAGCTGAA |
| | | AAAGCCAGGCGAGACCGTGAAGATCTCCTGCAAGGCCAGCGGCTACA |
| | | CTTTCAAGAATTATGGCATGAACTGGGTGAAGCAGGCCCCCGGCAAG |
| | | GGGCTGAAGTGGATGGGGTGGATTAACACCTATACCGGCCAGCCCAT |
| | | TTACGCCAACGATTTCAAGGGGAGATTCGCCTTTAGCCTGGAAACCA |
| | | GCGCGAGCACCGCCTATCTGCAGATCAACAATCTTAAGAATGAGGAT |
| | | ACAGCCACCTACTTTTGCGCCCGGGATTGGGGCCCATACTGGGGCCA |
| | | GGGCACCCTGGTGATCGTGTCCGCTTCCCCCAATTCTGCCTCCCACAG |
| | | CGGCAGCGCCCCACAGACTAGCTCCGCCCCCGGTTCCCAGAATATCA |
| | | TGATGACACAGAGCCCTTCCAGCCTGGCCGTGTCTGCCGGCGAAAAG |
| | | GTGACCGTGAACTGCAAGTCTAGCCAGTCTGTGCTGTACAGCTCCAAT |
| | | CAGATGAACTATCTGGCCTGGTACCAGCAGAAGCCTGGCCAGAGCCC |
| | | TAAGCTGCTGATTTACTGGGCCAGCACACGCGAGTCTGGGGTCCCTG |
| | | ACAGATTTACCGGTAGCGGAAGCGGCACAGATTTCACACTGACAATC |
| | | TCTTCTGTGCAGACCGAAGACCTCGCAGTGTACTACTGTCTGCAGTAT |
| | | CTGAGCTCTTGGACTTTCGGCGGGGGGACCAAGCTGGAAATTAAGGG |
| | | CGGATCTAGTCGGTCATCCTCCAGTGGTGGTGGGGGTAGCGGGGGAG |
| | | GCGGTGCTGGACAGCCGCTGCAAGAGCGGGCACAAGCCTGGGGGGA |
| | | AAGACTCAGGGCCAGAATGGAAGAAATGGGATCAAGAACAAGGGAC |
| | | AGGCTCGATGAGGTGAAGGAGCAAGTAGCAGAGGTTCGAGCAAAGC |
| | | TGGAAGAACAAGCTCAGCAGATTAGATTGCAAGCAGAGGCGGCTCAA |
| | | GCACGACTCAAGAGCAGGTTCGAGCCATTGGCTGAAGATATGCAGCG |
| | | ACAATGGGCTGGTCAGGTTGAGAAAGTCCAAGCGGCGGAAGGGACC |
| | | AGTGCAGCACCGGTACCATCCGACAATCATGGTGGAAGCAGTAGATC |
| | | ATCCAGTTCTGGTGGGGGAGGATCCGGGGGCGGGGGTGAGGTTCAGC |
| | | TCCTTGAATCAGGCGGAGGGCTCGTCCAACCAGGCGGATCTTTGCGG |
| | | CTGTCATGTGCCGCATCCGGATTCACGTTCTCCTCCTTTCCGATGGCAT |
| | | GGGTGAGGCAAGCTCCTGGAAAAGGTCTCGAGTGGGTATCCACCATA |
| | | AGTACTTCTGGCGGGCGCACTTACTACAGGGATAGTGTGAAGGGAAG |
| | | GTTTACCATTTCCAGAGATAACTCAAAGAATACCCTGTACCTGCAAAT |
| | | GAACTCCCTCAGGGCTGAAGATACGGCTGTGTACTACTGTGCTAAATT |
| | | TCGACAATACTCAGGTGGGTTTGACTATTGGGGCCAGGGGACACTGG |
| | | TGACTGTGTCATCAAGTCCCAATTCCGCCTCACATAGCGGAAGTGCCC |
| | | CACAAACTTCCTCCGCGCCAGGTTCTCAAGACATTCAATTGACGCAGC |
| | | CGAATAGCGTCTCTACGAGCTTGGGGAGTACCGTTAAGCTTTCATGTA |
| | | CTCTTTCCTCCGGTAACATAGAGAACAACTACGTGCATTGGTACCAAC |
| | | TCTACGAGGGCAGGTCTCCAACGACTATGATTTACGATGACGATAAA |
| | | CGACCTGATGGGGTCCCTGACCGCTTTTCAGGAAGCATTGACAGAAG |
| | | CTCTAATTCCGCGTTTTTGACTATCCACAACGTAGCGATTGAAGATGA |
| | | AGCAATTTACTTCTGTCATTCCTATGTATCCAGCTTTAACGTCTTCGGA |
| | | GGTGGAACTAAGTTGACGGTACTCCGAGGGGGAGGTGGGTCTCACCA |
| | | CCATCACCACCACCATCAT |

Three constructs, CM04A (αPEG/αCD3), CM04P (αPEG/αEPCAM), intended as a negative control), and CM04B (mutApoE3 Lipid Binding Domain/αCD3), were expressed from 50 ml Chinese Hamster Ovary (CHO) suspension cells (Evitria). The supernatant was filtered and applied to a 1 ml His-Excel column using an AKTA Purifier 100. A step gradient protocol was used to elute the protein in a buffer containing 20 mM sodium phosphate, 300 mM sodium chloride and up to 500 mM Imidazole, pH 7.4. The protein peak of interest was pooled, buffer exchanged into PBS and validated using SDS-page and an anti-His tag HRP Western blot. Concentrations were calculated using a Nanodrop spectrophotometer.

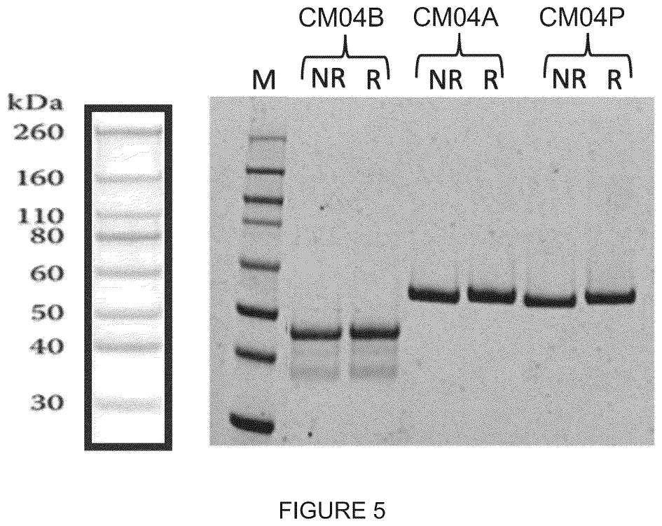

Each of the three protein constructs was then run on an SDS-PAGE gel for analysis and to separate based on their predicted molecular weights (FIG. 5). The estimated molecular weights of reagents CM04A (59.1 kDa), CM04P (58.9 kDa), and CM04B (43.6 kDa) align well with the observed results from the gel analysis. Additionally, a high level of purity (>90%) was observed for all proteins.

### Example 2: Binding of Multi-Specific Reagents to Jurkat T Cells (Supernatant)

Each multi-specific reagent was then screened for binding to Jurkat T cells. Jurkat T cells were plated into individual wells of a 96-well plate (˜100,000 cells per well in 200 ul ImmunoCult™-XF T Cell Expansion Medium (StemCell Technologies)). 20 μl of CHO culture supernatant containing expressed multi-specific engager proteins CM04A, CM04P, or CM04B were added to separate wells. As a control, CHO cell culture medium alone was added. Following incubation for two hours, cells were washed and stained in 50 μl FACS buffer (PBS (Gibco)/2% FBS (LabTech)) containing a 1/100 dilution of an APC-conjugated anti-His tag secondary antibody (BioLegend). Cells were washed again and resuspended in 100 μl FACS buffer plus 0.1 μg/ml DAPI live/dead staining dye (Roche). Cell samples were run on a MACSQuant® Analyzer 10 Flow Cytometer and analyzed using the FlowJo (v10) software.

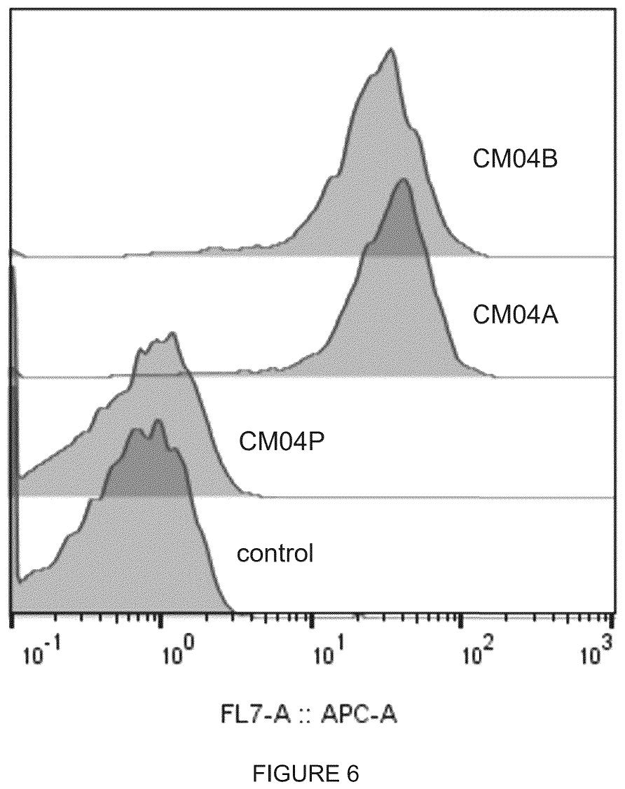

The histogram plots show APC fluorescent labelling of live (DAPI negative) cells following flow cytometry analysis, with binding to Jurkat T cells evident for CM04A and CM04B supernatant treatment (which both contain an anti-CD3 domain), but not for the CM04P supernatant (which contains domains that are irrelevant for T cell binding) (FIG. 6).

### Example 3: Binding of Multi-Specific Reagents to PEG Using ELISA

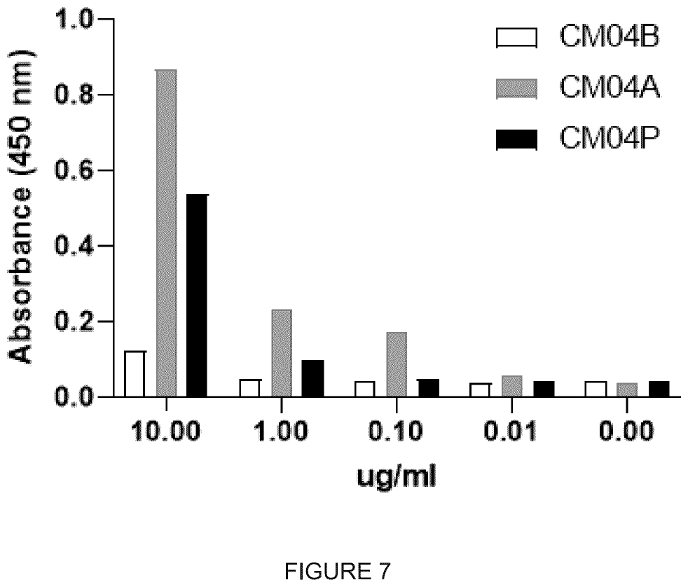

The constructs were each then screened for their ability to bind to PEG. 1 μg/ml streptavidin was coated onto an ELISA plate, followed by 1 mg/ml of PEG-Biotin [Alpha-Biotin-Omega-Carboxysuccinimidyl Ester Poly(ethylene glycol)]. Multi-specific engager proteins were added at concentrations between 10 μg/ml and 0.01 μg/ml and binding was detected using an anti-HIS tag-HRP antibody. As shown in FIG. 7, PEG-binding was observed for CM04A and CM04P (both containing an anti-PEG domain), but not for CM04B (without the anti-PEG domain).

### Example 4: Multi-Specific Reagent-Mediated Delivery of mRNA to T Cells

The delivery efficiency of mRNA to T cells was determined. mRNA-containing Lipid Nanoparticles (mRNA-LNPs) containing CleanCap EGFP mRNA (5moU) (Trilink BioTechnologies) were generated using a NanoAssemblr Spark instrument (Precision Nanosystems) according to the manufacturer's protocol.

Primary human PBMCs were isolated from blood and maintained in cell culture medium along with 10 ng/ml recombinant human IL-2 (Miltenyi).

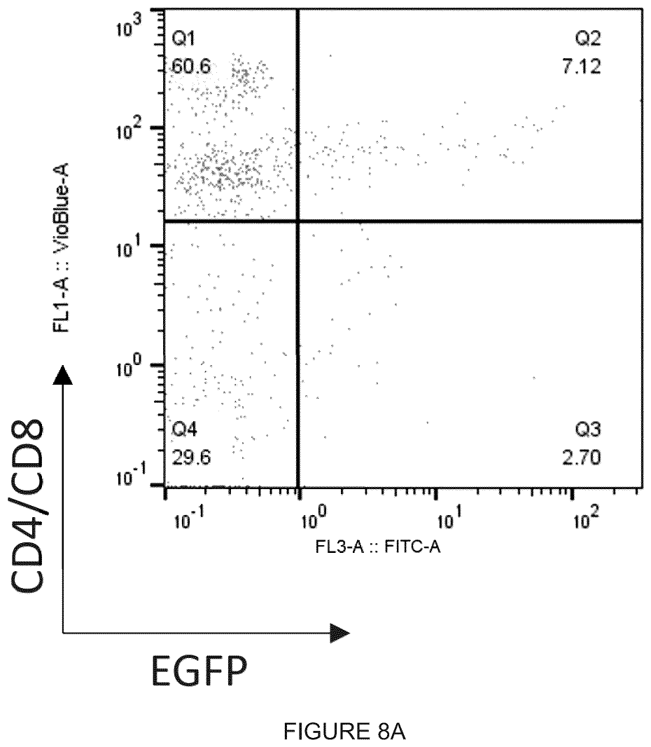

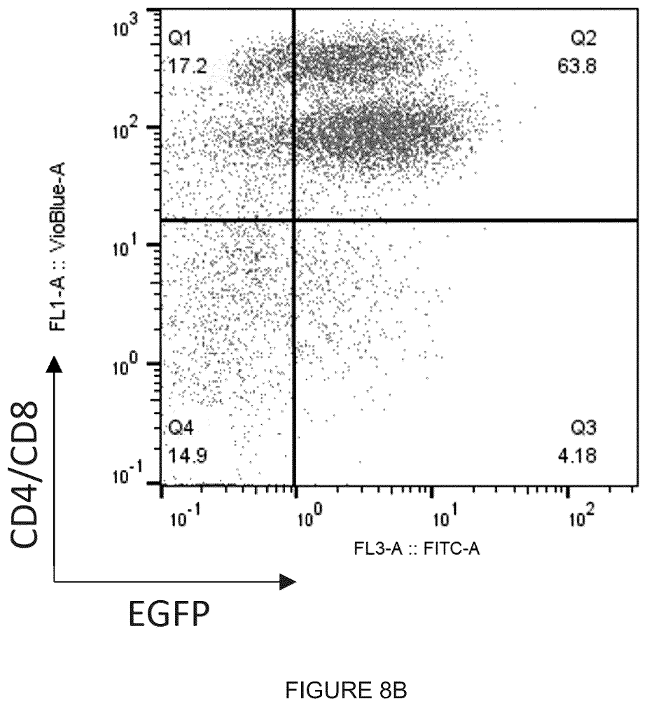

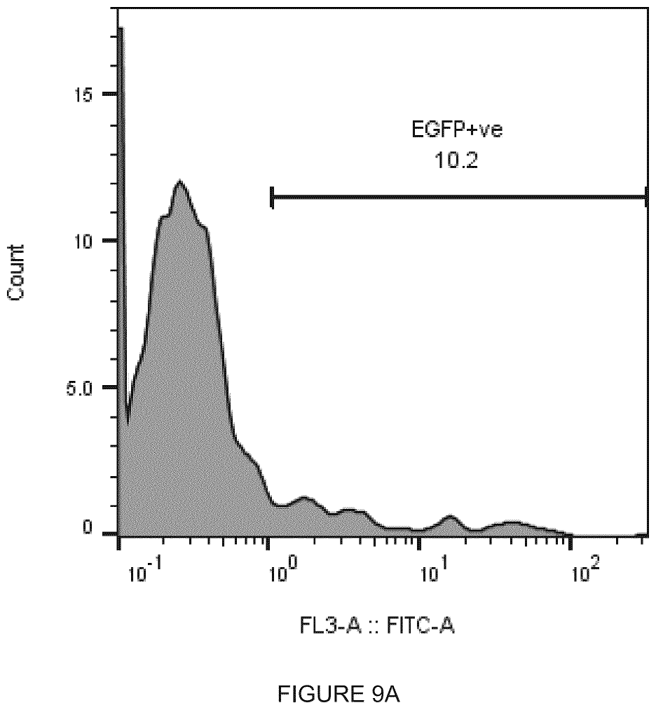

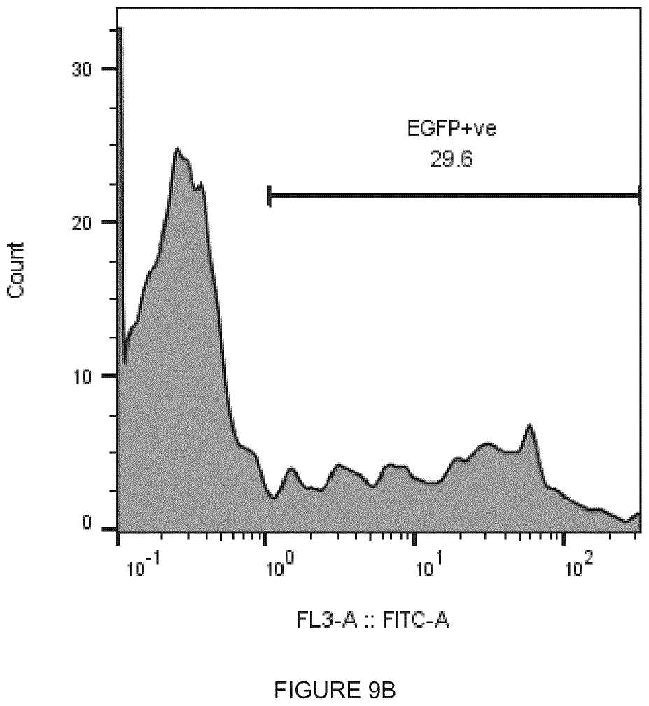

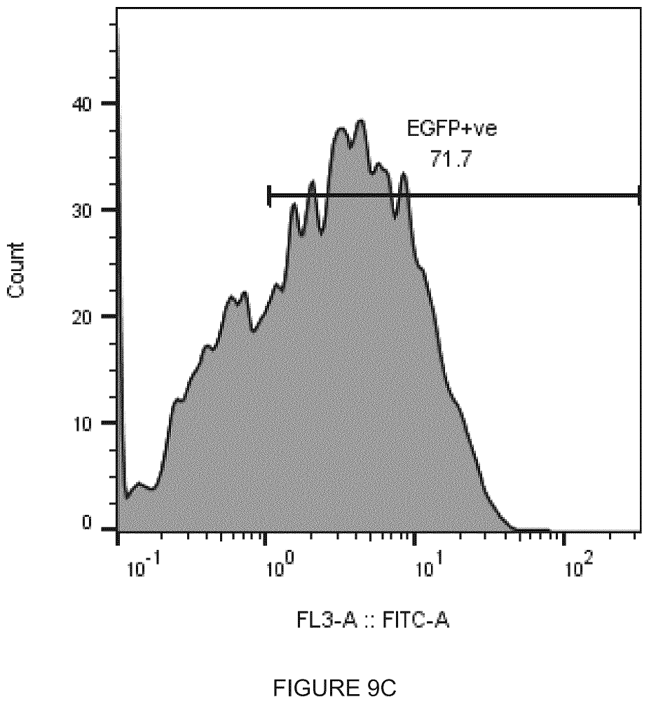

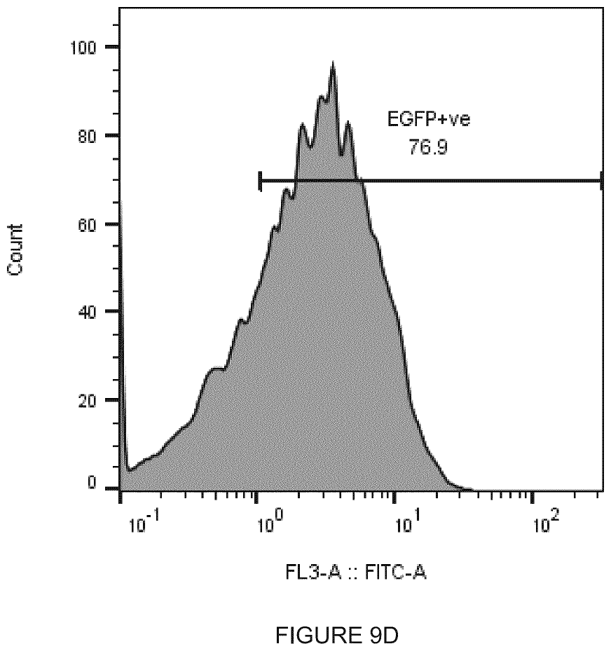

Cells were plated at approximately 100,000 cells/well and treated with 2 μl (approximately 0.1 μg) of EGFP mRNA-LNPs per well, plus 1 μg/ml multi-specificity reagent (CM04B). Approximately 96 hours after treatment, PBMCs were stained with Fixable Viability Dye eFluor™ 780, plus BV421-labelled CD4 and CD8 antibodies. The population of EGFP-positive T cells was determined using Flow Cytometry (FIGS. 8A-8B). In tangent, EGFP fluorescence was also screened within CD4/CD8+ T cell populations from starting PBMCs treated with LNPs alone or LNPs plus CM04P, CM04A, or CM04B protein (FIGS. 9A-9D). The plots show specific T cell-uptake of EGFP mRNA-LNPs mediated by CM04A- and CM04B-treatment (both containing anti-CD3 domain), but not by CM04P-treatment (containing a domain irrelevant for T cell-binding). Together, the data indicated that multi-specific engagers possessing an anti-CD3 binding domain significantly enhance LNP uptake specifically into primary human T cells.

### Example 5: Reduced Expression and APOE Blocking in a Liver Cell Line

Next, the multi-specific reagents were screened for activity in the presence of an ApoE3 protein. ApoE3 is a bi-modular protein with one domain primarily mediating lipid binding and another primarily mediating binding to low density lipoprotein receptor (LDLR) on cell surface. One major route for the uptake of LNPs into liver cells is through ApoE3 bound simultaneously to both lipids on LNPs and LDLR on liver cells, and subsequent internalization into cells. De-coupling ApoE lipid binding and LDLR-binding can potentially block liver cell uptake of LNPs.

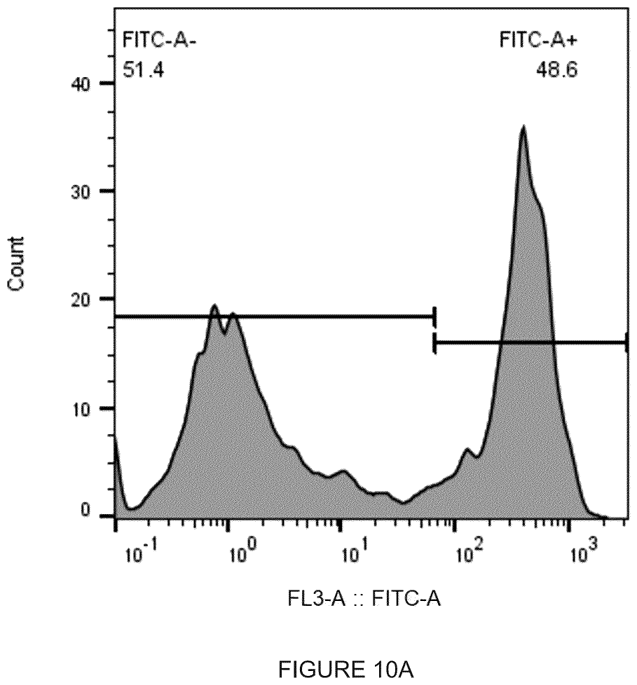

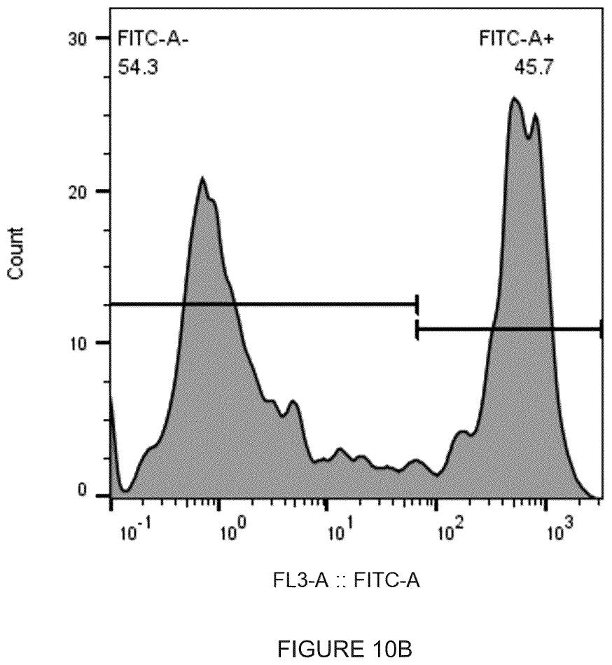

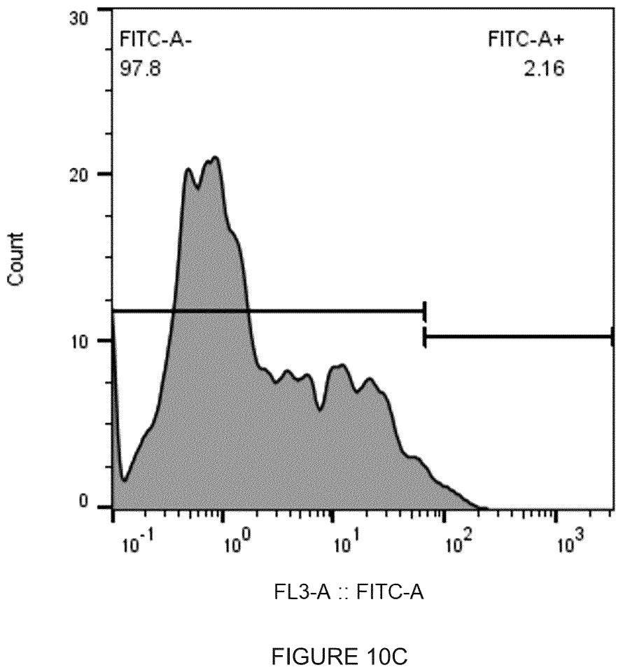

24 hours prior to treatment with EGFP mRNA-LNPs, human liver cancer cell line HepG2 cells were plated at ˜50,000 cells/well into wells of a 96-well plate and cultured in DMEM medium (Gibco) supplemented with 10% FBS (LabTech) 2 μl of EGFP mRNA-LNPs (approximately 0.1 μg mRNA) were incubated with 10 μl of 0.1 mg/ml purified dual-specific engager protein, or with 10 μl PBS, for −2 hours prior to cell treatment. Additionally, recombinant ApoE3 protein was added to all wells to a final concentration of 1 μg/ml. 24 h post-treatment with specified reagents, HepG2 cells were stained with DAPI live/dead dye, and the cell populations were then analyzed by flow cytometry (FIGS. 10A-10C). As shown, CM04B (containing ApoE3-LBD only) strongly inhibits LNP-uptake into HepG2 cell line mediated by ApoE3, whereas such effect is not evident for CM04A (containing no domain from ApoE3). Together, this data indicated that the multi-specific reagent including an ApoE3 lipid binding domain (LBD) is capable of inhibiting LNP uptake into a HepG2 liver cell line.

### Example 6: Multi-Specific Proteins with Increased Affinities

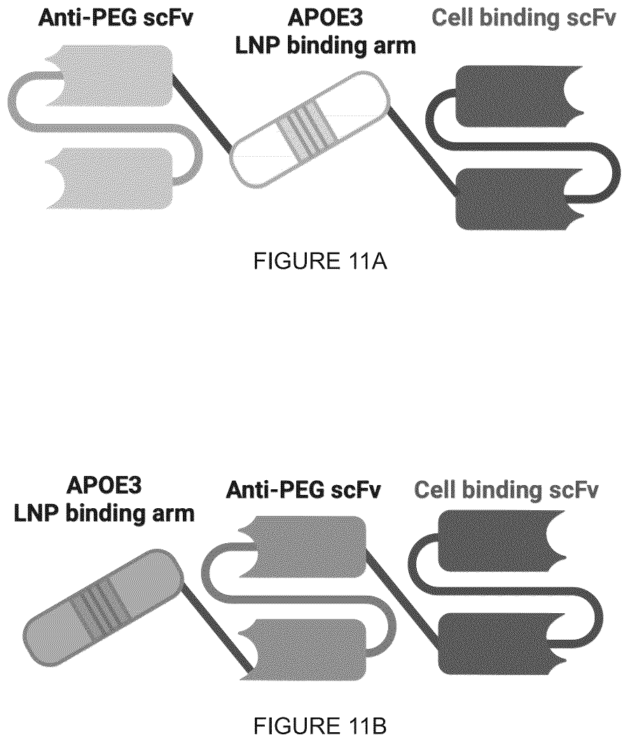

Variations to the multi-specific molecules were made to produce enhanced affinities. Non-limiting examples of the enhanced multi-specific molecules are as shown in FIGS. 11A-11D. The increased affinity dual targeting reagents include a PEG binding molecule fused to ApoE3 LBD which targets the LNP, this combination is additionally fused to a cell targeting antibody fragment.

Three constructs, including CM04A (αPEG/αCD3), CM04B (mutApoE3 Lipid Binding Domain/αCD3) and CM04C (αPEG/mutApoE3 Lipid Binding Domain/αCD3), were expressed from 100 ml CHO suspension cells (Evitria). The supernatant was filtered and applied to a 1 ml His-Excel column using an AKTA Purifier 100. A step gradient protocol was used to elute the protein in a buffer containing 20 mM sodium phosphate, 300 mM sodium chloride and up to 500 mM Imidazole, pH 7.4. The protein peak of interest was pooled, buffer exchanged into PBS and validated using SDS-page and an anti-His tag HRP Western blot. Concentrations were calculated using a Nanodrop spectrophotometer.

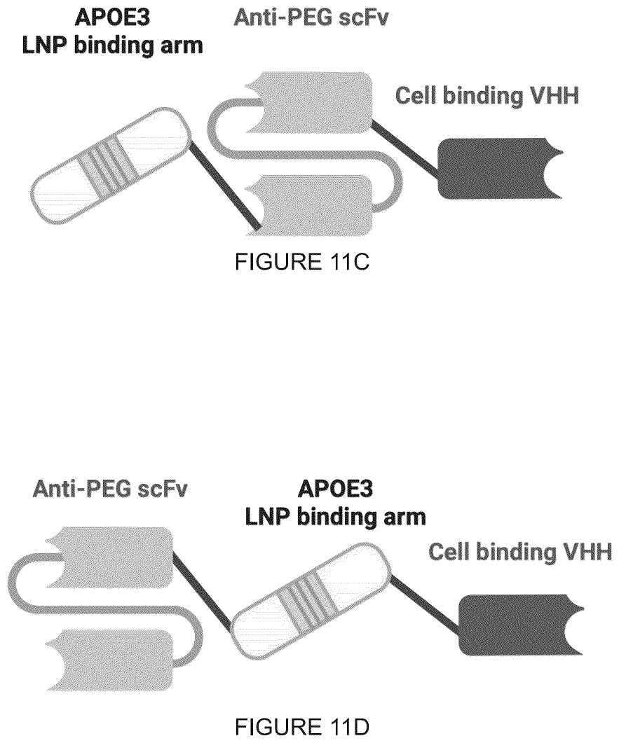

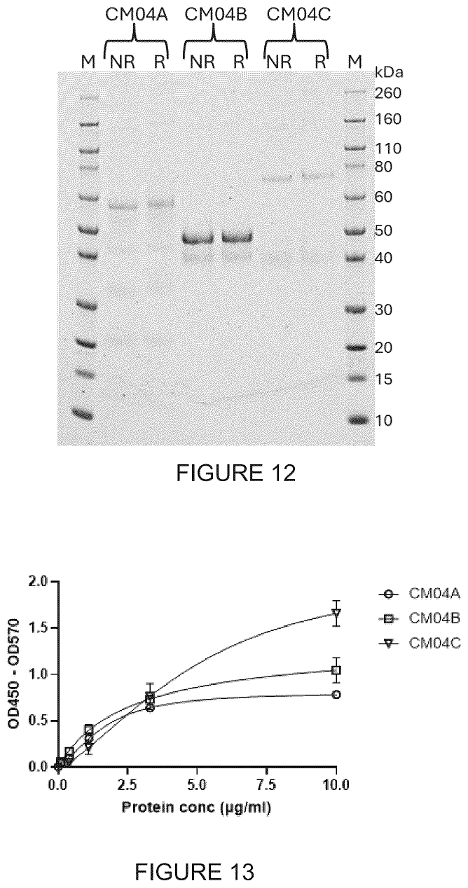

Each of the three protein constructs was then run on an SDS-PAGE gel for analysis, as well as to separate the proteins based on their predicted molecular weights (FIG. 12). The estimated molecular weights of reagents CM04A (59.1 kDa), CM04 B (43.6 kDa), and CM04C (71.6 kDa) align well with the observed results from the gel analysis. Additionally, a high level of purity (>90%) was observed for all proteins.

To measure the binding affinity of CM04A, CM04B and CM04C to LNP, an enzyme-linked immunosorbent assay (ELISA) was developed. A 96 well immunoassay plate (MaxiSorp™ flat-bottom, Fisher Scientific) was coated with 1 g/ml rabbit-anti-cholesterol polyclonal antibodies (Abbexa Ltd). LNPs (+LNP) or bovine serum albumin as negative control (−LNP ctl) were captured in different wells on the coated plate. Subsequently, CM04A, CM04B and CM04C between 10 g/ml and 0.1 g/ml were added to designated wells and the binding to captured LNPs or negative control was detected using an anti-HIS tag-HRP antibody. FIG. 13 displays the specific-binding to LNP of each protein at different concentrations as calculated by subtracting signals of (−LNP ctl) from those of (+LNP). As shown, CM04C which contains dual targeting domains to LNP, including a PEG binding fused to ApoE3-LBD, exhibited higher binding to LNP than CM04A (containing only PEG binding) and CM04B (containing only ApoE3-LBD).

### Example 7: Internalization Capacity of Multi-Specific Targeting Reagents

CHO suspension cells are utilized to generate multi-targeting reagents, which are subsequently purified using NiNTA affinity chromatography.

To assess internalization efficiency, T cell lines expressing CD3, CD5, and TRBC1 markers, as well as control cell lines lacking the target proteins, are employed. The measurement of internalization efficiency is quantified utilizing the conjugation of proteins with Zenon pHrodo iFL labeling reagent (ThermoFisher), following incubation with the target cells for a maximum of 24 hours. Next, the cells are stained with a live/dead viability dye and analyzed using a flow cytometer.

The multi-targeting reagents exhibit enhanced internalization, as evidenced by an increasing pH-dependent fluorescence signal over time when interacting with cells expressing the target antigen. In contrast, control multi-targeting reagents that do not bind to T cell markers show little internalization.

### Example 8: Dual Targeted L NPs Produce Active mRNA-Based CD19-CAR T Cells In Vitro

Primary human PBMCs are incubated with CD19-CAR mRNA-LNPs, with and without dual targeting reagents. The targeted LNPs efficiently deliver their mRNA cargo to the majority of T cells present in the culture. This successful delivery is confirmed by the surface expression of CD19-CAR on T cells following exposure to dual targeting reagent with CD19CAR/LNPs, as assessed using flow cytometry. In contrast, non-targeted LNPs generate much lower CAR expression.

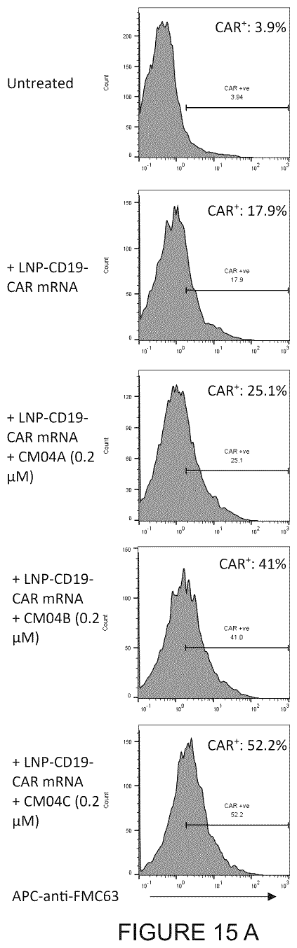

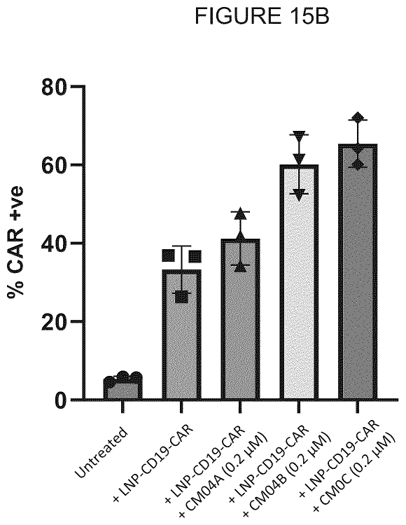

CD19-CAR mRNA expression was quantified followed mediation by LNP with and without dual targeting reagents in CD4+ and CD8+ T cells from three human donors (FIGS. 15A-15B). In particular, LNPs carrying 60 ng CD19-CAR-mRNA were incubated with CM04A, CM04B or CM04C at 0.2 M for 1 hr at room temperature. Subsequently, LNP alone or the mix of LNPs and various proteins were added to 1×10⁵ donor PBMCs for each treatment. The treated cells were cultured at 37° C., 5% CO₂ for 24 hrs. The cells were subsequently stained with FITC-anti-CD4/CD8 antibodies (BioLegend) and APC-anti-FMC63 antibody specific for the CAR (ACRO Biosystems). As shown, the exposure to dual targeting reagent—CM04A and CM04B significantly enhances the percentage of CAR+-T cells from all three donors, reaching 25-65%.

Furthermore, the CAR T cells generated through LNP-mediated delivery demonstrate effectiveness in targeting CD19-expressing cells in vitro. This efficacy is observed in a dose-dependent manner and is comparable to viral transduced CAR T cells.

### Example 9: Dual Targeted L NPs Produce mRNA-Based T Cell Expression In Vivo

Dual-specific targeting reagents that bind to mouse T cell markers TCR-β and CD3 are produced and purified. Next, mice are injected intravenously with LNPs with and without dual targeting reagents specific for T cells. Mice that receive intravenous injections of dual targeting reagents/LNPs containing luciferase mRNA (DT/LNP-Luc) exhibit significant luciferase activity in their splenic T cells. In contrast, mice that are injected with the LNP-Luc alone show much less luciferase activity in splenic T cells.

The utilization of bioluminescence imaging confirms the specific targeting of the spleen in animals treated with DT/LNP-Luc. Additionally, DT/LNP-Luc-treated animals exhibit reduced luciferase expression in the liver compared to LNP-Luc alone.

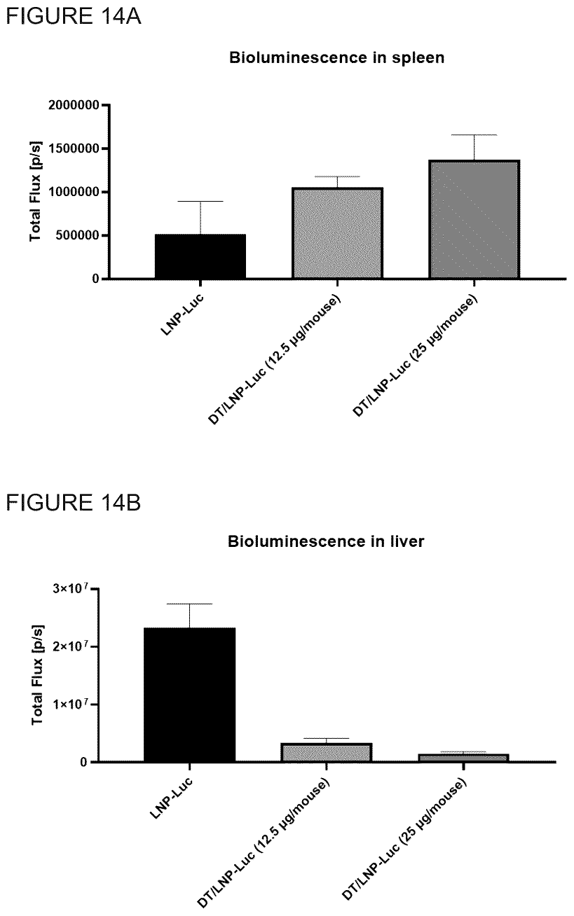

A surrogate dual-specific targeting reagent comprising ApoE3-LBD fused to a binder specific for mouse CD3 was produced and purified as described in Example 1. 1 g of LNP-Luc alone or 1 g of LNP-Luc plus surrogate dual-specific targeting reagent (DT/LNP-Luc) at 12.5 or 25 g dose were intravenously injected into the tail vein of female C57BL/6J mice. Three mice were included into each treatment group. Bioluminescence imaging was carried out on dissected spleen and liver at 24 hr post-injection. The dual-specific targeting reagent was shown to significantly enhance the luciferase signals in treated mouse spleen (FIG. 14A), while decreasing the signals in liver in comparison to LNP-Luc alone (FIG. 14B). The enhancing/decreasing effect is more profound when higher dose of dual-specific targeting reagent is used (25 g vs. 12.5 g).

### Example 10: Increased Avidity Multi-Targeting Reagents Will Enhance LNP Uptake into Primary Human T Cells and Will Inhibit LNP Uptake into Liver Cell Lines

Increased avidity dual targeting receptors (IADTRs) are created by fusing ApoE3 LBD to anti PEG single-chain variable fragments (scFvs) and binding domains that specifically recognize T cell markers (FIGS. 11A-11D). The production of (IADTRs) involves the generation of CHO suspension cells, and subsequent purification using NiNTA affinity chromatography.

Primary human PBMCs are isolated from blood samples and incubated with EGFP mRNA-LNPs both with and without IADTRs. The treated cells are analyzed using flow cytometry. When PBMCs are treated with LNPs alone, little expression of EGFP is observed. However, when IADTRs are added, a T cell subset of PBMCs displays significantly increased EGFP fluorescence.

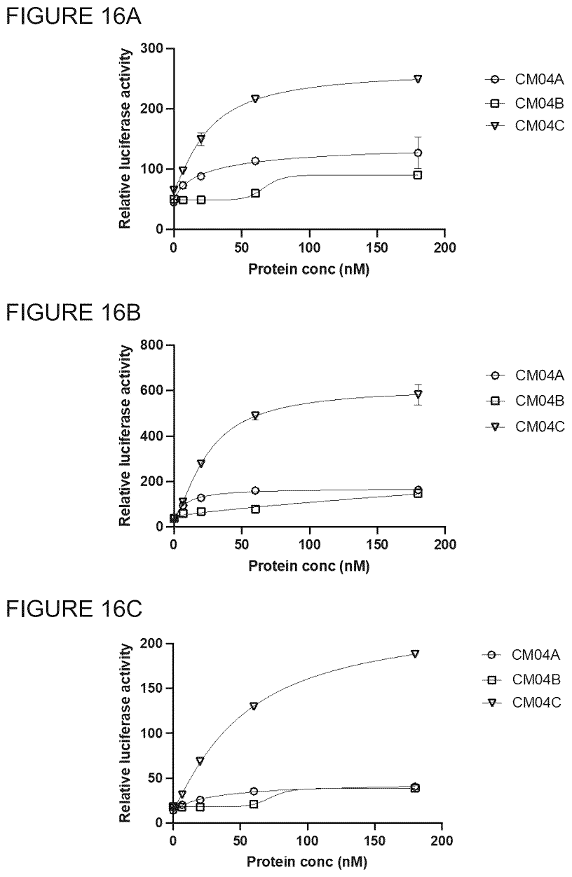

Additionally, fresh T cells were isolated from the blood of three different donors. The T cells were treated with firefly luciferase mRNA encapsulated in LNPs (LNPs-Fluc, TriLink) in combination with dual-targeting reagents (CM04A containing only PEG-binding scFvs or CM04B containing only ApoE3-LBD), or IADTRs (CM04C containing PEG-binding scFvs fused to ApoE3-LBD). In particular, LNPs-Fluc carrying 40 ng Fluc-mRNA were incubated with CM04A, CM04B or CM04C in concentrations between 6.7 to 180 nM for 1 hr at room temperature. Subsequently, (LNPs-Fluc) alone or the mix of (LNPs-Fluc) and CM04A, CM04B or CM04C were added to 80,000 donor T cells for each treatment. The treated cells were cultured at 37° C., 5% CO₂ for 24 hrs. The luciferase signals were assayed using ONE-Glo™ EX Luciferase Assay System (Promega) and detected on Varioskan LUX Multimode Microplate Reader (ThermoFisher). FIGS. 16A-16C display the ‘Relative Luciferase Activity’ calculated as fold change of signals from treated T cells vs. background reading from untreated cells in function of concentrations of various targeting reagents. As shown, all three tested reagents led to dose-dependent enhancement of LNPs-Fluc expression, but CM04C as IADTRs resulted in much higher expression than both CM04A and CM04B, showing 2.7 to 5.5-fold increase.

Moreover, for LNP-CD19-CAR mRNA expression in various donor-derived T cells, the exposure to IADTR—CM04C can significantly increase the percentage of CAR+-cells, in comparison to those treated with dual-targeting reagent—CM04A and CM04B (52-73% for CM04C vs 25-65% for CM04A/CM04B) (FIGS. 15A-15B).

HepG2 liver cancer cells are cultured in a medium containing ApoE3 and exposed to EGFP mRNA-LNPs with and without IADTRs. The treated cells are analyzed using flow cytometry. HepG2 cells treated with LNPs alone exhibit significant EGFP expression. Conversely, cells treated with IADTRs demonstrate significantly reduced EGFP fluorescence, indicating the impact of IADTRs on inhibiting ApoE3-mediated LNP uptake and subsequent EGFP expression in HepG2 cells.

With respect to the use of plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those of skill within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

Any of the features of an embodiment of the first through second aspects is applicable to all aspects and embodiments identified herein. Moreover, any of the features of an embodiment of the first through third aspects is independently combinable, partly, or wholly with other embodiments described herein in any way, e.g., one, two, or three or more embodiments may be combinable in whole or in part. Further, any of the features of an embodiment of the first through third aspects may be made optional to other aspects or embodiments.

What is claimed is:

1. A protein with multi-specificity, comprising:
(i) a first domain capable of binding to a therapeutic molecule; and
(ii) a second domain capable of binding to a protein, cell, or tissue,
wherein the therapeutic molecule is a lipid nanoparticle (LNP);
wherein the first domain has a high affinity for at least one of: polyethylene glycol (PEG), an ionizable lipid, a helper lipid, a cholesterol or a derivative of the cholesterol; and
wherein the first domain comprises an antibody or binding fragment thereof.

2. The protein of claim 1, wherein the LNP comprises at least one of: an ionizable lipid, a helper lipid, a cholesterol, a PEG-lipid, or any combination thereof.

3. The protein of claim 1, wherein the binding fragment of the first domain is a Fab′, F(ab′)2, single-chain variable fragment (scFv), diabody, minibody, nanobody, single domain antibody (sdAb), or a binding domain that is de novo designed by in silico methods.

4. The protein of claim 1, wherein the protein comprises a sequence with at least 80% identity to the sequence of any one of SEQ ID NO: 6, 16, 21, 38, 39, 43, 44, or any combination thereof.

5. A composition comprising a multi-specific protein and a lipid nanoparticle (LNP),
wherein the multi-specific protein comprises a first domain capable of binding the LNP; and a second domain capable of binding a protein, cell, or tissue;
wherein the first domain has an affinity for polyethylene glycol (PEG), an ionizable lipid, a helper lipid, a cholesterol or a derivative of the cholesterol,
wherein the first domain comprises an antibody or binding fragment thereof, and
wherein the LNP further comprises a payload that is a nucleic acid molecule.

6. The composition of claim 5, wherein the LNP comprises at least one of: an ionizable lipid, a helper lipid, a cholesterol, a PEG-lipid, or any combination thereof.

7. The composition of claim 5, wherein the payload is an mRNA or a DNA.

8. The composition of claim 7, wherein the mRNA or the DNA encodes for a chimeric antigen receptor (CAR) construct.

9. The composition of claim 5, wherein the first and/or second binding domain comprises an Fab′, F(ab′)2, single-chain variable fragment (scFv), diabody, minibody, nanobody, single domain antibody (sdAb), antibody variable (Fv) region-like polypeptide, or a binding domain de novo designed by in silico methods.

10. The composition of claim 6, wherein the helper lipid comprises phosphatidylserine, and wherein the multi-specific protein has an affinity for phosphatidylserine.

11. The composition of claim 5, wherein the second domain has an affinity for a cell surface antigen.

12. The composition of claim 11, wherein the cell surface antigen is any one of: a T cell receptor α subunit, a T cell receptor β subunit, a CD3, a CD4, a CD8, a CD5, a CD28, or any combination thereof.

13. The composition of claim 5, wherein the cell is any one of: an immune cell, a tumor cell, a lymphocyte, a T cell, a tumor infiltrating lymphocyte (TIL) cell, a natural killer (NK) cell, a CD8+ T cell, a CD4+ T cell, a regulatory T cell, a memory T cell, or any combination thereof.

14. The composition of claim 5, wherein the protein with multi-specificity further comprises an at least one linker.

15. The composition of claim 14, wherein the first domain is linked to the second domain via at least one linker.

16. The composition of claim 14, wherein the at least one linker comprises an at least one peptide linker;
optionally, wherein the at least one peptide linker comprises a sequence with at least 80% identity to the sequence of SEQ ID NO: 2.

17. The composition of claim 5, wherein the protein first domain comprises a sequence with at least 80% identity to the sequence of any one of SEQ ID NO: 6, 16, 21, 38, 39, 43, 44, or any combination thereof.

18. The composition of claim 5, wherein the multi-specific protein has three binding targets.

19. A method for treating a disease or disorder in a subject in need thereof, the method comprising administering to the subject the composition of claim 5.

20. A method of delivering a nucleic acid molecule to a target cell in a subject, comprising administering to the subject the composition of claim 5.
